(19) United States
(12) Patent Application Publication
(10) Pub. No.: US 2023/0383349 A1
(43) Pub. Date: Nov. 30, 2023

(54) METHODS OF ASSESSING RISK OF DEVELOPING A DISEASE

(71) Applicant: GENETIC TECHNOLOGIES LIMITED, Fitzroy (AU)

(72) Inventors: Kevin WONG, Carlton (AU); Nick MURPHY, Point Cook (AU); Gillian DITE, Thombury (AU); Aviv GAFNI, Caulfield North (AU); Richard ALLMAN, Manor Lakes (AU)

(73) Assignee: GENETIC TECHNOLOGIES LIMITED., Fitzroy, VI (AU)

(21) Appl. No.: 18/249,615

(22) PCT Filed: Oct. 19, 2021
(86) PCT No.: PCT/AU2021/051218

§ 371 Date: Apr. 19, 2023

(30) Foreign Application Priority Data
Oct. 20, 2020 (AU) 2020903793

(51) Int. Cl. C12Q 1/6883; G16B 20/20; G16H 50/20

(52) U.S. Cl. CPC C12Q 1/6883; G16B 20/20; C12Q 2600/106; C12Q 2600/118; C12Q 2600/156; G16H 50/20

(57) ABSTRACT

The present disclosure relates to methods and systems for assessing the risk of a human subject for developing a disease such as coronary artery disease, atrial fibrillation or Type 2 diabetes. These methods may be combined with the subjects clinical risk to improve risk analysis. Such methods may be used to assist decision making about appropriate therapeutic and monitoring regimens.

### FIELD OF THE INVENTION

The present disclosure relates to methods and systems for assessing the risk of a human subject for developing a disease such as coronary artery disease, atrial fibrillation or Type 2 diabetes. These methods may be combined with the subjects clinical risk to improve risk analysis. Such methods may be used to assist decision making about appropriate therapeutic and monitoring regimens.

### BACKGROUND OF THE INVENTION

The most common cardiovascular disease is coronary artery disease (CAD) which involves the reduction of blood flow to the heart muscle due to build-up of plaque (atherosclerosis) in the arteries of the heart. Clinical risk factors include high blood pressure, smoking, diabetes, lack of exercise, obesity, high blood cholesterol, poor diet, depression and excessive alcohol. A number of tests may help with diagnoses including: electrocardiogram, cardiac stress testing, coronary computed tomographic angiography, and coronary angiogram.

Atrial fibrillation (AF) is an abnormal heart rhythm (arrhythmia) characterized by the rapid and irregular beating of the atrial chambers of the heart. AF typically begins as short periods of abnormal beating, which become longer or continuous over time. It may also start as other forms of arrhythmia such as atrial flutter that then transform into AF. High blood pressure and valvular heart disease are the most common alterable risk factors for AF. Other heart-related risk factors include heart failure, coronary artery disease, cardiomyopathy, and congenital heart disease.

Type 2 diabetes (T2D), also known as adult-onset diabetes, is a form of diabetes that is characterized by high blood sugar, insulin resistance, and relative lack of insulin. Long-term complications from high blood sugar include heart disease, strokes, diabetic retinopathy which can result in blindness, kidney failure, and poor blood flow in the limbs which may lead to amputations. Type 2 diabetes primarily occurs as a result of obesity and lack of exercise.

The Framingham Risk Score is a gender-specific algorithm used to estimate the 10-year CAD risk of an individual. It is also used assessing risk of developing others diseases such as AF and T2D. The Framingham Risk Score was first developed based on data obtained from the Framingham Heart Study, to estimate the 10-year risk of developing coronary heart disease (Wilson et al., 1998). In order to assess the 10-year cardiovascular disease risk, cerebrovascular events, peripheral artery disease and heart failure were subsequently added as disease outcomes for the 2008 Framingham Risk Score, on top of coronary heart disease (D'Agonstino et al., 2008).

There is a need for improved methods of assessing an individuals risk of developing coronary artery disease, atrial fibrillation or Type 2 diabetes.

### SUMMARY OF THE INVENTION

In a first aspect, the present invention provides a method for assessing the risk of a human subject developing coronary artery disease, the method comprising performing a genetic risk assessment of the human subject, wherein the genetic risk assessment involves detecting, in a biological sample derived from the human subject, the presence of at least one polymorphisms associated with a risk of developing coronary artery disease, wherein the at least one polymorphism is selected from Table 1 and/or Table 2, or a single nucleotide polymorphism in linkage disequilibrium with one or more thereof.

In an embodiment of the above aspect, the method comprises detecting the presence of at least two, at least three, at least four, at least five, at least six, at least seven, at least eight, at least nine, at least ten, at least 20, at least 50, at least 100, at least 150, at least 200, at least 250, at least 300, at least 350, at least 400, at least 450, at least 500, at least 550, at least 600, at least 650, at least 700, at least 750, at least 800, at least 825, at least 850, or all of the polymorphism provided in Table 1, or a single nucleotide polymorphism in linkage disequilibrium with one or more thereof.

In an embodiment of the above aspect, the method comprises detecting the presence of at least two, at least three, at least four, at least five, at least six, at least seven, at least eight, at least nine, at least ten, at least 20, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000 or all of the polymorphism provided in Tables 1 and 2, or a single nucleotide polymorphism in linkage disequilibrium with one or more thereof.

In an embodiment of the above aspect, the method comprises detecting each of the polymorphisms provided in Table 1. In an embodiment, method further comprises detecting each of the polymorphisms provided in Table 2.

In another aspect, the present invention provides a method for assessing the risk of a human subject developing atrial fibrillation, the method comprising performing a genetic risk assessment of the human subject, wherein the genetic risk assessment involves detecting, in a biological sample derived from the human subject, the presence at least one polymorphisms associated with a risk of developing atrial fibrillation, wherein the at least one polymorphism is selected from Table 3, or a single nucleotide polymorphism in linkage disequilibrium with one or more thereof.

In an embodiment of the above aspect, the method comprises detecting the presence of at least two, at least three, at least four, at least five, at least six, at least seven, at least eight, at least nine, at least ten, at least 20, at least 50, at least 100, at least 125, at least 150, at least 175, at least 200, at least 225, at least 250, or all of the polymorphism provided in Table 3, or a single nucleotide polymorphism in linkage disequilibrium with one or more thereof.

In an embodiment of the above aspect, the method comprises detecting each of the polymorphism provided in Table 3.

In a further aspect, the present invention provides a method for assessing the risk of a human subject developing Type 2 diabetes, the method comprising performing a genetic risk assessment of the human subject, wherein the genetic risk assessment involves detecting, in a biological sample derived from the human subject, the presence at least one polymorphisms associated with a risk of developing Type 2 diabetes, wherein the at least one polymorphism is selected from Table 4, or a single nucleotide polymorphism in linkage disequilibrium with one or more thereof.

In an embodiment of the above aspect, the method comprises detecting the presence of at least two, at least three, at least four, at least five, at least six, at least seven, at least eight, at least nine, at least ten, at least 20, at least 30, at least 40, at least 50, at least 60, at least 70, at least 80, at least 85, or all of the polymorphism provided in Table 4, or a single nucleotide polymorphism in linkage disequilibrium with one or more thereof.

In an embodiment of the above aspect, the method comprises detecting each of the polymorphism provided in Table 4.

In an embodiment, the method further comprises
performing a clinical risk assessment of the human subject; and
combining the clinical risk assessment and the genetic risk assessment to obtain the risk of a human subject developing coronary artery disease, atrial fibrillation or Type 2 diabetes.

In an embodiment, the clinical risk assessment includes, but is not limited to, obtaining information from the subject on one or more or all of: age, gender, HDL-cholesterol level (mmol/L), LDL-cholesterol level (mmol/L), total cholesterol level, blood pressure (systolic and/or diastolic (mm Hg)), smoking status, have or has had diabetes, on hypertension medication, c-reactive protein levels, whether the subject's mother or father have had a heart attack (such by the age of 60), body mass index, ethnicity, measures of deprivation, family history, have or has had chronic kidney disease, and have or has had rheumatoid arthritis.

In an embodiment, the clinical risk assessment includes obtaining information from the subject on one or more or all of: age, gender, HDL-cholesterol level (mmol/L), LDL-cholesterol level (mmol/L), total cholesterol level, blood pressure (systolic and diastolic (mm Hg)), smoking status, have or has had diabetes and on hypertension medication.

In an embodiment, the clinical risk assessment is the Framingham score.

In an embodiment, when the disease is coronary artery disease, the clinical risk assessment is The American College of Cardiologists Pooled Cohort Equations (PCE).

In an embodiment, combining the clinical risk assessment and the genetic risk assessment comprises adding or multiplying the risk assessments.

In a further aspect, the present invention provides a method of determining the identity of the alleles of fewer than 100,000 polymorphisms in a human subject selected from the group of subjects consisting of humans in need of assessment for the risk of developing coronary artery disease to produce a polymorphic profile of the subject, comprising
(i) selecting for allelic identity analysis at least one polymorphism provided in Table 1 and/or Table 2, or a single nucleotide polymorphism in linkage disequilibrium with one or more thereof,
(ii) detecting, in a biological sample derived from the human subject, the polymorphisms, and
(iii) producing the polymorphic profile of the subject screening based on the identity of the alleles analysed in step (ii), wherein fewer than 100,000 polymorphisms are selected for allelic identity analysis in step (i) and the same fewer than 100,000 polymorphisms are analysed in step (ii).

In a further aspect, the present invention provides a method of determining the identity of the alleles of fewer than 100,000 polymorphisms in a human subject selected from the group of subjects consisting of humans in need of assessment for the risk of developing atrial fibrillation to produce a polymorphic profile of the subject, comprising
(i) selecting for allelic identity analysis at least one polymorphism provided in Table 3, or a single nucleotide polymorphism in linkage disequilibrium with one or more thereof,
(ii) detecting, in a biological sample derived from the human subject, the polymorphisms, and
(iii) producing the polymorphic profile of the subject screening based on the identity of the alleles analysed in step (ii), wherein fewer than 100,000 polymorphisms are selected for allelic identity analysis in step (i) and the same fewer than 100,000 polymorphisms are analysed in step (ii).

In a further aspect, the present invention provides a method of determining the identity of the alleles of fewer than 100,000 polymorphisms in a human subject selected from the group of subjects consisting of humans in need of assessment for the risk of developing Type 2 diabetes to produce a polymorphic profile of the subject, comprising
(i) selecting for allelic identity analysis at least one polymorphism provided in Table 4, or a single nucleotide polymorphism in linkage disequilibrium with one or more thereof,
(ii) detecting, in a biological sample derived from the human subject, the polymorphisms, and
(iii) producing the polymorphic profile of the subject screening based on the identity of the alleles analysed in step (ii), wherein fewer than 100,000 polymorphisms are selected for allelic identity analysis in step (i) and the same fewer than 100,000 polymorphisms are analysed in step (ii).

In an embodiment of the above three aspects, where relevant, fewer than 100,000 polymorphisms, fewer than 50,000 polymorphisms, fewer than 40,000 polymorphisms, fewer than 30,000 polymorphisms, fewer than 20,000 polymorphisms, fewer than 10,000 polymorphisms, fewer than 7,500 polymorphisms, fewer than 5,000 polymorphisms, fewer than 4,000 polymorphisms, fewer than 3,000 polymorphisms, fewer than 2,000 polymorphisms, fewer than 1,000 polymorphisms, fewer than 900 polymorphisms, fewer than 800 polymorphisms, fewer than 700 polymorphisms, fewer than 600 polymorphisms, fewer than 500 polymorphisms, fewer than 400 polymorphisms, fewer than 300 polymorphisms, fewer than 200 polymorphisms, or fewer than 100 polymorphisms, are selected for allelic identity.

In an embodiment, the polymorphism(s) in linkage disequilibrium has linkage disequilibrium above 0.9. In an embodiment, the polymorphism(s) in linkage disequilibrium has linkage disequilibrium above 0.95. In an embodiment, the polymorphism(s) in linkage disequilibrium has linkage disequilibrium above 0.99. In another embodiment, the polymorphism(s) in linkage disequilibrium has linkage disequilibrium of 1.

In an embodiment, the risk assessment produces a score and the method further comprises comparing the score to a predetermined threshold, wherein if the score is at, or above, the threshold the subject is assessed at being at risk of developing coronary artery disease, atrial fibrillation or Type 2 diabetes.

In a further aspect, the present invention provides a method for determining the need for routine diagnostic testing of a human subject for a coronary artery disease, atrial fibrillation or Type 2 diabetes, the method comprising assessing the risk of the subject for developing coronary artery disease, atrial fibrillation or Type 2 diabetes using a method of the invention.

In another aspect, the present invention provides a method of screening for coronary artery disease, atrial fibrillation or Type 2 diabetes in a human subject, the method comprising assessing the risk of the subject for developing coronary artery disease, atrial fibrillation or Type 2 diabetes using a method of the invention, and routinely screening for coronary artery disease, atrial fibrillation or Type 2 diabetes in the subject if they are assessed as having a risk for developing coronary artery disease, atrial fibrillation or Type 2 diabetes.

In an embodiment of the above two aspects, for coronary artery disease the screening involves conducting one or more of an electrocardiogram (ECG), an exercise stress test, a nuclear stress test, a cardiac catheterization and angiogram, or a cardiac CT scan.

In an embodiment of the above two aspects, for atrial fibrillation disease the screening involves conducting an electrocardiogram (ECG).

In an embodiment of the above two aspects, for Type 2 diabetes the screening involves analysing one or more of blood glucose levels, urine glucose levels, glycated hemoglobin (HbA1c) levels, fructosamine levels or glucose tolerance of the subject.

In an aspect, the present invention provides a method for determining the need of a human subject for prophylactic anti-coronary artery disease therapy, anti-atrial fibrillation therapy or anti-Type 2 diabetes therapy comprising assessing the risk of the subject for developing coronary artery disease, atrial fibrillation or Type 2 diabetes using a method of the invention.

In yet a further aspect, the present invention provides a method for preventing or reducing the risk of coronary artery disease, atrial fibrillation or Type 2 diabetes in a human subject, the method comprising assessing the risk of the subject for developing coronary artery disease, atrial fibrillation or Type 2 diabetes using a method of the invention, and if they are assessed as having a risk for developing coronary artery disease, atrial fibrillation or Type 2 diabetes administering anti-coronary artery disease therapy, anti-atrial fibrillation therapy or anti-Type 2 diabetes therapy, respectively.

In a further aspect, the present invention provides an anti-coronary artery disease therapy, anti-atrial fibrillation therapy or anti-Type 2 diabetes therapy for use in preventing coronary artery disease, atrial fibrillation or Type 2 diabetes, respectively, in a human subject at risk thereof, wherein the subject is assessed as having a risk for developing coronary artery disease, atrial fibrillation or Type 2 diabetes using a method of the invention.

In an embodiment, the anti-coronary artery disease therapy is selected from, but not limited to, cholesterol lowering medication such as a statin, blood thinning medication such as aspirin, warfarin or rivaroxaban, a β-blocker, nitrates, or a calcium channel blocker.

In an embodiment, the anti-atrial fibrillation therapy is selected from, but not limited to, cardioversion, a n-blocker, a calcium channel blocker, blood thinning medication such as warfarin, aspirin or rivaroxaban, or an antiarrhythmic drug such as quinidine, flecainide, propafenone, sotalol, dofetilide. or amiodar.

In an embodiment, the anti-Type 2 diabetes therapy is selected from, but not limited to, metformin, insulin, a sulfonylurea such as glimepiride, glyburide or glipizide, a meglitinide such as prandin or starlix, a thiazolidinedione such as rosiglitazone or pioglitazone, a DPP-4 inhibitor such as sitagliptin, saxagliptin, linagliptin or alogliptin, a GLP-1 receptor agonist such as exenatide, liraglutide, lixisenatide, albiglutide or dulaglutide, and an SGLT2 inhibitor such as forxiga, invokana or jardiance.

In another aspect, the present invention provides a method for stratifying a group of human subject's for a clinical trial of a candidate therapy, the method comprising assessing the individual risk of the subject's for developing coronary artery disease, atrial fibrillation or Type 2 diabetes a method of the invention, and using the results of the assessment to select subject's more likely to be responsive to the therapy.

Also provides a kit comprising at least two sets of primers for amplifying two or more nucleic acids, wherein the two or more nucleic acids comprise a polymorphism selected from any one of Tables 1 to 4, or any combinations thereof such as Tables, 1, 3 and 4, or Tables 1 and 2, or a single nucleotide polymorphism in linkage disequilibrium with one or more thereof.

In a further aspect, provided is a genetic array comprising at least two sets of probes for hybridising to two or more nucleic acids, wherein the two or more nucleic acids comprise a polymorphism selected from any one of Tables 1 to 4, or any combinations thereof such as Tables, 1, 3 and 4, or Tables 1 and 2, or a single nucleotide polymorphism in linkage disequilibrium with one or more thereof.

In an aspect, the present invention provides a computer implemented method for assessing the risk of a human subject developing coronary artery disease, atrial fibrillation or Type 2 diabetes, the method operable in a computing system comprising a processor and a memory, the method comprising:
receiving genetic risk data for the human subject, wherein the genetic risk data was obtained by a method of the invention;
processing the data to obtain the risk of a human subject developing coronary artery disease, atrial fibrillation or Type 2 diabetes; and
outputting the risk of a human subject developing coronary artery disease, atrial fibrillation or Type 2 diabetes.

In a further aspect, the present invention provides a computer implemented method for assessing the risk of a human subject developing coronary artery disease, atrial fibrillation or Type 2 diabetes, the method operable in a computing system comprising a processor and a memory, the method comprising:
receiving clinical risk data and genetic risk data for the human subject, wherein the clinical risk data and genetic risk data were obtained by a method of the invention;
processing the data to combine the clinical risk data with the genetic risk data to obtain the risk of a human subject developing coronary artery disease, atrial fibrillation or Type 2 diabetes; and
outputting the risk of a human subject developing coronary artery disease, atrial fibrillation or Type 2 diabetes.

In another aspect, the present invention provides a system for assessing the risk of a human subject developing coronary artery disease, atrial fibrillation or Type 2 diabetes comprising:
system instructions for performing a genetic risk assessment of the human subject using a method of the invention; and
system instructions to obtain the risk of a human subject developing coronary artery disease, atrial fibrillation or Type 2 diabetes.

In another aspect, the present invention provides a system for assessing the risk of a human subject developing coronary artery disease, atrial fibrillation or Type 2 diabetes comprising:
system instructions for performing a clinical risk assessment and a genetic risk assessment of the human subject using a method of the invention; and
system instructions for combining the clinical risk assessment and the genetic risk assessment to obtain the risk of a human subject developing coronary artery disease, atrial fibrillation or Type 2 diabetes.

In an embodiment, the risk data for the subject is received from a user interface coupled to the computing system. In another embodiment, the risk data for the subject is received from a remote device across a wireless communications network. In another embodiment, the user interface or remote device is a SNP array platform. In another embodiment, outputting comprises outputting information to a user interface coupled to the computing system. In another embodiment, outputting comprises transmitting information to a remote device across a wireless communications network.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying FIGURES.

### BRIEF DESCRIPTION OF THE ACCOMPANYING DRAWINGS

FIG. 1: Workflow of the study described in the Examples.

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., coronary artery disease, atrial fibrillation and Type 2 diabetes analysis, treatment and prevention, molecular genetics, bioinformatics and biochemistry).

Unless otherwise indicated, the molecular and statistical techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

It is to be understood that this disclosure is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, optionally include plural referents unless the content clearly dictates otherwise. Thus, for example, reference to “a probe” optionally includes a plurality of probe molecules; similarly, depending on the context, use of the term “a nucleic acid” optionally includes, as a practical matter, many copies of that nucleic acid molecule.

As used herein, the term “about”, unless stated to the contrary, refers to +/−10%, more preferably +/−5%, more preferably +/−1%, of the designated value.

The term “and/or”, e.g., “X and/or Y” shall be understood to mean either “X and Y” or “X or Y” and shall be taken to provide explicit support for both meanings or for either meaning.

As used herein, the term “coronary artery disease” refers to the narrowing or blockage of the coronary arteries limiting blood flow to the heart, usually caused by hardening or clogging of the arteries due to the buildup of cholesterol and fatty deposits (called plaques) on the inner walls of the arteries. Symptoms include, but are not limited to, angina, cold sweats, dizziness, light-headedness, nausea or a feeling of indigestion, neck pain, shortness of breath especially with activity, and sleep disturbances. Coronary artery disease is also known in the art as “coronary heart disease” and “atherosclerotic heart disease”.

As used herein, the term “atrial fibrillation” refers to an irregular, typically rapid, heart rate that occurs when the two upper chambers of your heart experience chaotic electrical signals. The result is a fast and irregular heart rhythm. The heart rate in atrial fibrillation generally ranges from 100 to 175 beats a minute. Symptoms include, but are not limited to, general fatigue, rapid and irregular heartbeat, fluttering or thumping in the chest, dizziness, shortness of breath and anxiety, weakness, faintness or confusion, and fatigue when exercising.

As used herein, the term “Type 2 diabetes” or T2D refers to a chronic condition that affects the way the body processes blood sugar (glucose). With type 2 diabetes, the body either does not produce enough insulin, or it resists insulin. Symptoms of Type 2 diabetes include increased thirst, frequent urination, hunger, fatigue and blurred vision. In some cases, there may be no symptoms.

A “polymorphism” is a locus that is variable; that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations).

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc.

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. An allele “positively” correlates with a trait when it is linked to it and when presence of the allele is an indicator that the trait or trait form will occur in an individual comprising the allele. An allele “negatively” correlates with a trait when it is linked to it and when presence of the allele is an indicator that a trait or trait form will not occur in an individual comprising the allele. The term “risk allele” is used in the context of the present disclosure to refer to an allele indicating a genetic propensity to susceptibility to coronary artery disease, atrial fibrillation or Type 2 diabetes. A subject can be homozygous, heterozygous or null for a particular risk allele.

A marker polymorphism or allele is “correlated” or “associated” with a specified phenotype (coronary artery disease, atrial fibrillation or Type 2 diabetes susceptibility, etc.) when it can be statistically linked (positively or negatively) to the phenotype. Methods for determining whether a polymorphism or allele is statistically linked are known to those in the art. That is, the specified polymorphism(s) occurs more commonly in a case population (e.g., coronary artery disease, atrial fibrillation or Type 2 diabetes patients) than in a control population (e.g., individuals that do not have coronary artery disease, atrial fibrillation or Type 2 diabetes patients, respectively). This correlation is often inferred as being causal in nature, but it need not be—simple genetic linkage to (association with) a locus for a trait that underlies the phenotype is sufficient for correlation/association to occur.

The phrase “linkage disequilibrium” (LD) is used to describe the statistical correlation between two neighbouring polymorphic genotypes. Typically, LD refers to the correlation between the alleles of a random gamete at the two loci, assuming Hardy-Weinberg equilibrium (statistical independence) between gametes. LD is quantified with either Lewontin's parameter of association (D′) or with Pearson correlation coefficient (r) (Devlin and Risch, 1995). Two loci with a LD value of 1 are said to be in complete LD. At the other extreme, two loci with a LD value of 0 are termed to be in linkage equilibrium. Linkage disequilibrium is calculated following the application of the expectation maximization algorithm (EM) for the estimation of haplotype frequencies (Slatkin and Excoffier, 1996). LD values according to the present disclosure for neighbouring genotypes/loci are selected above 0.5, more preferably, above 0.6, still more preferably, above 0.7, preferably, above 0.8, more preferably above 0.9, ideally about 1.0. Many of the SNPs in linkage disequilibrium with the SNPs of the present disclosure that are described herein have LD values of 0.9 or 1.

Another way one of skill in the art can readily identify SNPs in linkage disequilibrium with the SNPs of the present disclosure is determining the LOD score for two loci. LOD stands for “logarithm of the odds”, a statistical estimate of whether two genes, or a gene and a disease gene, are likely to be located near each other on a chromosome and are therefore likely to be inherited. A LOD score of between about 2-3 or higher is generally understood to mean that two genes are located close to each other on the chromosome. Thus, in an embodiment, LOD values according to the present disclosure for neighbouring genotypes/loci are selected at least above 2, at least above 3, at least above 4, at least above 5, at least above 6, at least above 7, at least above 8, at least above 9, at least above 10, at least above 20 at least above 30, at least above 40, at least above 50.

In another embodiment, SNPs in linkage disequilibrium with the SNPs of the present disclosure can have a specified genetic recombination distance of less than or equal to about 20 centimorgan (cM) or less. For example, 15 cM or less, 10 cM or less, 9 cM or less, 8 cM or less, 7 cM or less, 6 cM or less, 5 cM or less, 4 cM or less, 3 cM or less, 2 cM or less, 1 cM or less, 0.75 cM or less, 0.5 cM or less, 0.25 cM or less, or cM or less. For example, two linked loci within a single chromosome segment can undergo recombination during meiosis with each other at a frequency of less than or equal to about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.75%, about 0.5%, about 0.25%, or about 0.1% or less.

In another embodiment, SNPs in linkage disequilibrium with the SNPs of the present disclosure are within at least 100 kb (which correlates in humans to about 0.1 cM, depending on local recombination rate), at least 50 kb, at least 20 kb or less of each other.

One exemplary approach for the identification of surrogate markers for a particular SNP involves a simple strategy that presumes that SNPs surrounding the target SNP are in linkage disequilibrium and can therefore provide information about disease susceptibility. Potentially surrogate markers can therefore be identified from publicly available databases, such as HAPMAP, by searching for SNPs fulfilling certain criteria which have been found in the scientific community to be suitable for the selection of surrogate marker candidates.

“Allele frequency” refers to the frequency (proportion or percentage) at which an allele is present at a locus within an individual, within a line or within a population of lines. For example, for an allele “A,” diploid individuals of genotype “AA,” “Aa,” or “aa” have allele frequencies of 1.0, 0.5, or 0.0, respectively. One can estimate the allele frequency within a line or population (e.g., cases or controls) by averaging the allele frequencies of a sample of individuals from that line or population. Similarly, one can calculate the allele frequency within a population of lines by averaging the allele frequencies of lines that make up the population.

In an embodiment, the term “allele frequency” is used to define the minor allele frequency (MAF). MAF refers to the frequency at which the least common allele occurs in a given population.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal position or region. For example, a polymorphic locus is a position or region where a polymorphic nucleic acid, trait determinant, gene or marker is located. In a further example, a “gene locus” is a specific chromosome location (region) in the genome of a species where a specific gene can be found.

A “marker,” “molecular marker” or “marker nucleic acid” refers to a nucleotide sequence or encoded product thereof (e.g., a protein) used as a point of reference when identifying a locus or a linked locus. A marker can be derived from genomic nucleotide sequence or from expressed nucleotide sequences (e.g., from an RNA, nRNA, mRNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. For example, a marker locus can be used to monitor segregation of alleles at a locus, such as a quantitative trait locus (QTL), that are genetically or physically linked to the marker locus. Thus, a “marker allele,” alternatively an “allele of a marker locus” is one of a plurality of polymorphic nucleotide sequences found at a marker locus in a population that is polymorphic for the marker locus.

In one embodiment, the present disclosure provides marker loci correlating with a phenotype of interest, e.g., coronary artery disease, atrial fibrillation or Type 2 diabetes. Each of the identified markers is expected to be in close physical and genetic proximity (resulting in physical and/or genetic linkage) to a genetic element, e.g., a QTL that contributes to the relevant phenotype. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of allele specific hybridization (ASH), detection of single nucleotide extension, detection of amplified variable sequences of the genome, detection of self-sustained sequence replication, detection of simple sequence repeats (SSRs), detection of single nucleotide polymorphisms (SNPs), or detection of amplified fragment length polymorphisms (AFLPs).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An “amplicon” is an amplified nucleic acid, e.g., a nucleic acid that is produced by amplifying a template nucleic acid by any available amplification method (e.g., PCR, LCR, transcription, or the like).

A specified nucleic acid is “derived from” a given nucleic acid when it is constructed using the given nucleic acid's sequence, or when the specified nucleic acid is constructed using the given nucleic acid.

A “gene” is one or more sequence(s) of nucleotides in a genome that together encode one or more expressed molecules, e.g., an RNA, or polypeptide. The gene can include coding sequences that are transcribed into RNA which may then be translated into a polypeptide sequence, and can include associated structural or regulatory sequences that aid in replication or expression of the gene.

A “genotype” is the genetic constitution of an individual (or group of individuals) at one or more genetic loci. Genotype is defined by the allele(s) of one or more known loci of the individual, typically, the compilation of alleles inherited from its parents.

A “haplotype” is the genotype of an individual at a plurality of genetic loci on a single DNA strand. Typically, the genetic loci described by a haplotype are physically and genetically linked, i.e., on the same chromosome strand.

A “set” of markers, probes or primers refers to a collection or group of markers probes, primers, or the data derived therefrom, used for a common purpose (e.g., assessing an individuals risk of developing coronary artery disease, atrial fibrillation or Type 2 diabetes). Frequently, data corresponding to the markers, probes or primers, or derived from their use, is stored in an electronic medium. While each of the members of a set possess utility with respect to the specified purpose, individual markers selected from the set as well as subsets including some, but not all of the markers, are also effective in achieving the specified purpose.

The polymorphisms and genes, and corresponding marker probes, amplicons or primers described above can be embodied in any system herein, either in the form of physical nucleic acids, or in the form of system instructions that include sequence information for the nucleic acids. For example, the system can include primers or amplicons corresponding to (or that amplify a portion of) a gene or polymorphism described herein. As in the methods above, the set of marker probes or primers optionally detects a plurality of polymorphisms in a plurality of said genes or genetic loci. Thus, for example, the set of marker probes or primers detects at least one polymorphism in each of these genes, or any other polymorphism, gene or locus defined herein. Any such probe or primer can include a nucleotide sequence of any such polymorphism or gene, or a complementary nucleic acid thereof, or a transcribed product thereof (e.g., a nRNA or mRNA form produced from a genomic sequence, e.g., by transcription or splicing).

As used herein, “Receiver operating characteristic curves” refer to a graphical plot of the sensitivity vs. (1−specificity) for a binary classifier system as its discrimination threshold is varied. The ROC can also be represented equivalently by plotting the fraction of true positives (TPR=true positive rate) vs. the fraction of false positives (FPR=false positive rate). Also known as a Relative Operating Characteristic curve, because it is a comparison of two operating characteristics (TPR & FPR) as the criterion changes. ROC analysis provides tools to select possibly optimal models and to discard suboptimal ones independently from (and prior to specifying) the cost context or the class distribution. Methods of using in the context of the disclosure will be clear to those skilled in the art.

As used herein, the term “combining the genetic risk assessment with the clinical risk assessment to obtain the risk” refers to any suitable mathematical analysis relying on the results of the two assessments. For example, the results of the clinical risk assessment and the genetic risk assessment may be added, more preferably multiplied.

As used herein, the terms “routinely screening for coronary artery disease, atrial fibrillation or Type 2 diabetes” and “more frequent screening” are relative terms, and are based on a comparison to the level of screening recommended to a subject who has no identified risk of developing coronary artery disease, atrial fibrillation or Type 2 diabetes.

### Genetic Risk Assessment

In an embodiment, the methods of the present disclosure relate to assessing the risk of a subject for developing coronary artery disease, atrial fibrillation or Type 2 diabetes by performing a genetic risk assessment.

The genetic risk assessment is performed by analysing the genotype of the subject at one or more loci for single nucleotide polymorphisms.

The present invention provides a method for assessing the risk of a human subject developing coronary artery disease, the method comprising performing a genetic risk assessment of the human subject, wherein the genetic risk assessment involves detecting, in a biological sample derived from the human subject, the presence of at least one polymorphisms associated with a risk of developing coronary artery disease, wherein the at least one polymorphism is selected from Table 1, or a single nucleotide polymorphism in linkage disequilibrium with one or more thereof. In an embodiment, the method comprises detecting the presence of at least two, at least three, at least four, at least five, at least six, at least seven, at least eight, at least nine, at least ten, at least 20, at least 50, at least 100, at least 150, at least 200, at least 250, at least 300, at least 350, at least 400, at least 450, at least 500, at least 550, at least 600, at least 650, at least 700, at least 750, at least 800, at least 825, at least 850, or all of the polymorphism provided in Table 1, or a single nucleotide polymorphism in linkage disequilibrium with one or more thereof. In embodiment, the method comprises detecting each of the polymorphisms provided in Table 1.

TABLE 1
Polymorphisms associated with coronary artery disease. A1 is the risk allele.

| CHR | SNP | A1 | A2 | A1_freq | A2_freq | p_value | OR |
|---|---|---|---|---|---|---|---|
| 1 | rs60363208 | A | G | 0.174 | 0.826 | 1.65E−04 | 0.949441 |
| 1 | rs2843151 | C | T | 0.2131 | 0.7869 | 3.76E−05 | 1.049207 |
| 1 | rs2279677 | T | C | 0.02474 | 0.97526 | 0.00118 | 1.109926 |
| 1 | rs5024246 | A | G | 0.3529 | 0.6471 | 3.86E−04 | 1.03831 |
| 1 | rs2493292 | T | C | 0.1394 | 0.8606 | 1.15E−04 | 1.057464 |
| 1 | rs228690 | T | C | 0.0781 | 0.9219 | 6.62E−04 | 0.942976 |
| 1 | rs17882988 | A | G | 0.1574 | 0.8426 | 6.34E−04 | 0.956832 |
| 1 | rs2999867 | T | C | 0.2066 | 0.7934 | 1.16E−04 | 0.950723 |
| 1 | rs1538534 | G | T | 0.05134 | 0.94866 | 2.14E−04 | 0.929226 |
| 1 | rs7528228 | C | T | 0.02257 | 0.97743 | 8.99E−05 | 1.073389 |
| 1 | rs3008423 | A | C | 0.2109 | 0.7891 | 2.40E−04 | 0.957774 |
| 1 | rs17461632 | G | A | 0.09445 | 0.90555 | 0.001198 | 1.075264 |
| 1 | rs16823188 | C | T | 0.1797 | 0.8203 | 0.00108 | 1.040768 |
| 1 | rs35267671 | C | T | 0.4113 | 0.5887 | 1.88E−06 | 0.953997 |
| 1 | rs11809423 | T | C | 0.03723 | 0.96277 | 3.69E−04 | 1.086748 |
| 1 | rs1034268 | A | G | 0.3295 | 0.6705 | 1.02E−04 | 1.03874 |
| 1 | rs323716 | T | C | 0.3831 | 0.6169 | 2.78E−05 | 1.04182 |
| 1 | rs11211062 | A | G | 0.1685 | 0.8315 | 4.53E−05 | 1.051309 |
| 1 | rs55884940 | A | G | 0.2498 | 0.7502 | 2.27E−04 | 1.045192 |
| 1 | rs35615859 | C | T | 0.01815 | 0.98185 | 0.001114 | 0.886775 |
| 1 | rs41292511 | A | G | 0.1627 | 0.8373 | 0.001038 | 0.957055 |
| 1 | rs11206510 | C | T | 0.1882 | 0.8118 | 2.34E−08 | 0.92819 |
| 1 | rs7523242 | T | C | 0.1827 | 0.8173 | 0.001167 | 1.03956 |
| 1 | rs11591147 | T | G | 0.0175 | 0.9825 | 7.47E−06 | 0.773753 |
| 1 | rs6663252 | C | T | 0.1261 | 0.8739 | 7.56E−04 | 0.957125 |
| 1 | rs35905526 | A | G | 0.2952 | 0.7048 | 0.001108 | 1.043321 |
| 1 | rs17114046 | G | A | 0.09239 | 0.90761 | 6.06E−14 | 0.88259 |
| 1 | rs61772626 | G | A | 0.123 | 0.877 | 9.61E−06 | 1.067481 |
| 1 | rs1202812 | T | C | 0.3675 | 0.6325 | 4.79E−04 | 1.036719 |
| 1 | rs2716122 | C | A | 0.2048 | 0.7952 | 7.20E−04 | 1.038905 |
| 1 | rs6684353 | G | A | 0.3007 | 0.6993 | 1.16E−04 | 0.958985 |
| 1 | rs72671369 | A | G | 0.01842 | 0.98158 | 8.08E−04 | 1.078226 |
| 1 | rs12041665 | C | T | 0.06793 | 0.93207 | 6.44E−04 | 1.054119 |
| 1 | rs72675374 | T | C | 0.04645 | 0.95355 | 3.58E−04 | 1.100318 |
| 1 | rs11578253 | C | T | 0.3993 | 0.6007 | 5.62E−05 | 1.040446 |
| 1 | rs116029121 | A | C | 0.008523 | 0.991477 | 7.82E−04 | 1.169956 |
| 1 | rs2182148 | A | G | 0.02605 | 0.97395 | 4.52E−04 | 0.86811 |
| 1 | rs4970829 | A | G | 0.07186 | 0.92814 | 2.09E−06 | 0.907185 |
| 1 | rs72703203 | A | G | 0.04183 | 0.95817 | 1.41E−05 | 0.84143 |
| 1 | rs7528419 | G | A | 0.2218 | 0.7782 | 1.97E−23 | 0.891785 |
| 1 | rs655246 | A | G | 0.4709 | 0.5291 | 2.84E−05 | 0.959943 |
| 1 | rs35267391 | A | G | 0.01433 | 0.98567 | 7.16E−04 | 0.873763 |
| 1 | rs114382843 | C | T | 0.04283 | 0.95717 | 6.25E−04 | 0.882952 |
| 1 | rs114804066 | G | A | 0.01033 | 0.98967 | 1.32E−04 | 0.839733 |
| 1 | rs17026168 | A | G | 0.05899 | 0.94101 | 2.05E−04 | 1.059545 |
| 1 | rs76940009 | C | A | 0.04974 | 0.95026 | 9.11E−04 | 1.058581 |
| 1 | rs67224956 | C | T | 0.168 | 0.832 | 5.62E−04 | 0.959731 |
| 1 | rs6696923 | G | A | 0.3739 | 0.6261 | 0.001038 | 1.032149 |
| 1 | rs78685532 | C | T | 0.4456 | 0.5544 | 7.47E−04 | 0.968627 |
| 1 | rs41308401 | G | A | 0.04579 | 0.95421 | 4.34E−04 | 1.106094 |
| 1 | rs1722784 | A | G | 0.4902 | 0.5098 | 5.79E−05 | 0.961134 |
| 1 | rs77919400 | G | A | 0.02692 | 0.97308 | 6.28E−04 | 1.15454 |
| 1 | rs1568485 | C | T | 0.323 | 0.677 | 8.95E−04 | 1.037191 |
| 1 | rs3790514 | C | T | 0.1601 | 0.8399 | 4.00E−05 | 0.946268 |
| 1 | rs4845606 | A | G | 0.1334 | 0.8666 | 1.42E−04 | 0.947598 |
| 1 | rs35717427 | A | G | 0.1227 | 0.8773 | 3.15E−04 | 0.947828 |
| 1 | rs7549250 | C | T | 0.4197 | 0.5803 | 8.45E−09 | 1.054695 |
| 1 | rs78204256 | A | G | 0.03568 | 0.96432 | 6.90E−05 | 1.154601 |
| 1 | rs4845695 | G | A | 0.3643 | 0.6357 | 1.59E−05 | 1.042953 |
| 1 | rs74520550 | A | C | 0.2056 | 0.7944 | 3.73E−04 | 0.95079 |
| 1 | rs11589479 | A | G | 0.1626 | 0.8374 | 4.68E−04 | 1.050118 |
| 1 | rs10489836 | T | C | 0.03145 | 0.96855 | 3.77E−04 | 1.132503 |
| 1 | rs2789422 | A | G | 0.418 | 0.582 | 2.46E−06 | 0.95379 |
| 1 | rs59317225 | A | G | 0.167 | 0.833 | 6.78E−04 | 0.961316 |
| 1 | rs72702156 | A | G | 0.115 | 0.885 | 9.04E−04 | 1.050089 |
| 1 | rs756199 | G | A | 0.2076 | 0.7924 | 6.67E−04 | 1.037476 |
| 1 | rs114453840 | T | C | 0.03083 | 0.96917 | 9.53E−04 | 1.106063 |
| 1 | rs2820312 | A | G | 0.3413 | 0.6587 | 3.52E−05 | 1.042473 |
| 1 | rs56323810 | A | G | 0.07157 | 0.92843 | 0.001159 | 0.934837 |
| 1 | rs12088135 | T | C | 0.03516 | 0.96484 | 7.44E−04 | 0.906106 |
| 1 | rs67116579 | C | T | 0.188 | 0.812 | 8.90E−04 | 0.958949 |
| 1 | rs6660415 | C | T | 0.1549 | 0.8451 | 9.27E−04 | 0.959878 |
| 1 | rs72738491 | G | A | 0.03064 | 0.96936 | 7.41E−05 | 1.119652 |
| 1 | rs3816991 | C | T | 0.3649 | 0.6351 | 0.001224 | 1.042509 |
| 1 | rs1024065 | C | T | 0.1554 | 0.8446 | 8.87E−05 | 0.946451 |
| 1 | rs904324 | G | A | 0.09448 | 0.90552 | 5.32E−05 | 0.919394 |
| 1 | rs2291832 | G | A | 0.2849 | 0.7151 | 2.28E−12 | 0.925464 |
| 1 | rs17537624 | A | G | 0.4027 | 0.5973 | 6.94E−04 | 1.03751 |
| 1 | rs12047290 | C | T | 0.1167 | 0.8833 | 9.33E−04 | 0.956714 |
| 1 | rs2574878 | C | T | 0.1611 | 0.8389 | 7.81E−04 | 1.04036 |
| 1 | rs2144300 | C | T | 0.3929 | 0.6071 | 0.001147 | 1.031699 |
| 1 | rs6663784 | A | G | 0.1203 | 0.8797 | 3.74E−04 | 1.045848 |
| 1 | rs655555 | A | C | 0.3711 | 0.6289 | 5.19E−04 | 1.033242 |
| 1 | rs74865714 | C | T | 0.03839 | 0.96161 | 0.001011 | 0.897191 |
| 1 | rs12121819 | T | C | 0.3636 | 0.6364 | 0.001206 | 0.968692 |
| 1 | rs116731816 | A | G | 0.02372 | 0.97628 | 6.68E−04 | 1.146288 |
| 1 | rs61855662 | T | C | 0.1872 | 0.8128 | 1.56E−04 | 0.940721 |
| 1 | rs12354406 | G | A | 0.1611 | 0.8389 | 5.58E−04 | 1.046711 |
| 2 | rs13413321 | T | G | 0.4332 | 0.5668 | 5.56E−04 | 1.032755 |
| 2 | rs2709437 | C | T | 0.4726 | 0.5274 | 3.24E−04 | 0.966196 |
| 2 | rs116274618 | T | C | 0.03184 | 0.96816 | 6.66E−04 | 0.88797 |
| 2 | rs72773351 | T | C | 0.05696 | 0.94304 | 1.23E−04 | 0.885167 |
| 2 | rs35890230 | T | C | 0.2135 | 0.7865 | 6.85E−04 | 1.039936 |
| 2 | rs4464248 | A | G | 0.3045 | 0.6955 | 2.22E−04 | 1.0397 |
| 2 | rs35458571 | T | C | 0.2086 | 0.7914 | 6.63E−04 | 1.042211 |
| 2 | rs2123536 | T | C | 0.04638 | 0.95362 | 4.00E−04 | 1.058183 |
| 2 | rs75905766 | T | C | 0.01643 | 0.98357 | 0.001132 | 0.876184 |
| 2 | rs751098 | G | A | 0.3836 | 0.6164 | 7.68E−04 | 1.031975 |
| 2 | rs113780557 | C | T | 0.0229 | 0.9771 | 7.95E−05 | 0.913415 |
| 2 | rs550619 | G | A | 0.07831 | 0.92169 | 2.99E−04 | 0.945303 |
| 2 | rs1367117 | A | G | 0.3387 | 0.6613 | 1.10E−04 | 1.041997 |
| 2 | rs515135 | T | C | 0.1791 | 0.8209 | 3.09E−08 | 0.934729 |
| 2 | rs12477249 | A | G | 0.3385 | 0.6615 | 4.73E−05 | 1.040949 |
| 2 | rs219548 | A | C | 0.3176 | 0.6824 | 8.62E−04 | 1.034734 |
| 2 | rs6751857 | C | T | 0.4466 | 0.5534 | 1.72E−04 | 0.965336 |
| 2 | rs7561273 | A | G | 0.4702 | 0.5298 | 1.13E−04 | 1.036626 |
| 2 | rs75354157 | A | G | 0.02395 | 0.97605 | 7.13E−04 | 0.871838 |
| 2 | rs2303297 | T | C | 0.2771 | 0.7229 | 1.80E−04 | 1.041681 |
| 2 | rs13020831 | C | T | 0.4839 | 0.5161 | 7.80E−04 | 0.968398 |
| 2 | rs1123648 | T | C | 0.1921 | 0.8079 | 0.001164 | 0.964972 |
| 2 | rs12471686 | C | T | 0.1332 | 0.8668 | 6.14E−04 | 1.050071 |
| 2 | rs41360247 | C | T | 0.06325 | 0.93675 | 1.48E−07 | 0.89685 |
| 2 | rs4245791 | C | T | 0.3249 | 0.6751 | 6.14E−07 | 1.0535 |
| 2 | rs4148218 | A | G | 0.1811 | 0.8189 | 3.35E−05 | 0.950931 |
| 2 | rs582384 | C | A | 0.4505 | 0.5495 | 7.68E−04 | 0.967715 |
| 2 | rs72835349 | C | T | 0.03847 | 0.96153 | 0.001037 | 0.92083 |
| 2 | rs1990522 | T | C | 0.04693 | 0.95307 | 3.74E−04 | 0.936521 |
| 2 | rs6545968 | C | A | 0.3096 | 0.6904 | 5.42E−04 | 0.96551 |
| 2 | rs76083505 | T | C | 0.01126 | 0.98874 | 5.42E−04 | 0.892535 |
| 2 | rs56232353 | C | A | 0.4698 | 0.5302 | 0.00117 | 1.031295 |
| 2 | rs2287326 | T | G | 0.4272 | 0.5728 | 4.32E−04 | 0.966778 |
| 2 | rs1567229 | C | T | 0.1094 | 0.8906 | 5.11E−04 | 0.942882 |
| 2 | rs17721059 | T | C | 0.01808 | 0.98192 | 9.28E−04 | 0.844183 |
| 2 | rs72835953 | A | G | 0.2814 | 0.7186 | 8.81E−04 | 0.961924 |
| 2 | rs1561198 | T | C | 0.4615 | 0.5385 | 6.37E−10 | 1.060175 |
| 2 | rs1127974 | A | G | 0.1627 | 0.8373 | 1.61E−04 | 1.057035 |
| 2 | rs62159767 | C | A | 0.04151 | 0.95849 | 6.28E−04 | 0.858078 |
| 2 | rs79716828 | C | A | 0.03633 | 0.96367 | 6.95E−05 | 1.13127 |
| 2 | rs76197902 | G | A | 0.02181 | 0.97819 | 1.58E−04 | 1.168042 |
| 2 | rs6761276 | T | C | 0.4197 | 0.5803 | 7.34E−04 | 1.033634 |
| 2 | rs6721463 | G | A | 0.08846 | 0.91154 | 0.001167 | 0.959693 |
| 2 | rs11898671 | C | A | 0.1543 | 0.8457 | 5.20E−05 | 0.951786 |
| 2 | rs75149973 | G | T | 0.0225 | 0.9775 | 2.56E−04 | 1.160155 |
| 2 | rs7564469 | C | T | 0.1552 | 0.8448 | 2.06E−05 | 1.053987 |
| 2 | rs17740744 | G | A | 0.07743 | 0.92257 | 3.25E−09 | 1.112356 |
| 2 | rs12467931 | G | A | 0.3373 | 0.6627 | 1.51E−05 | 1.045533 |
| 2 | rs16825205 | C | T | 0.03588 | 0.96412 | 5.62E−04 | 1.072885 |
| 2 | rs1950047 | C | T | 0.4119 | 0.5881 | 1.59E−04 | 1.037599 |
| 2 | rs13012311 | T | C | 0.306 | 0.694 | 6.01E−05 | 1.040618 |
| 2 | rs16822558 | G | A | 0.3487 | 0.6513 | 6.87E−04 | 1.033527 |
| 2 | rs13006913 | G | A | 0.2946 | 0.7054 | 6.29E−04 | 0.964678 |
| 2 | rs1990760 | C | T | 0.3898 | 0.6102 | 5.28E−05 | 0.960878 |
| 2 | rs16849273 | G | A | 0.1767 | 0.8233 | 3.56E−04 | 0.957305 |
| 2 | rs3769243 | G | A | 0.2817 | 0.7183 | 0.00115 | 1.041293 |
| 2 | rs6708900 | T | C | 0.3053 | 0.6947 | 5.80E−04 | 0.964764 |
| 2 | rs62188269 | T | C | 0.1931 | 0.8069 | 4.93E−04 | 0.960658 |
| 2 | rs3100025 | T | C | 0.006226 | 0.993774 | 6.79E−04 | 1.147267 |
| 2 | rs17241582 | G | A | 0.2051 | 0.7949 | 0.001152 | 0.961417 |
| 2 | rs75324624 | C | T | 0.04961 | 0.95039 | 6.77E−05 | 1.127512 |
| 2 | rs6709162 | T | C | 0.353 | 0.647 | 1.29E−04 | 1.038572 |
| 2 | rs4303700 | A | G | 0.2309 | 0.7691 | 4.43E−04 | 1.04357 |
| 2 | rs6725887 | C | T | 0.1277 | 0.8723 | 9.51E−18 | 1.142119 |
| 2 | rs3731695 | T | C | 0.4495 | 0.5505 | 5.67E−05 | 1.039598 |
| 2 | rs11686036 | A | G | 0.4458 | 0.5542 | 6.89E−07 | 1.049042 |
| 2 | rs144923024 | A | G | 0.03193 | 0.96807 | 3.94E−04 | 1.114647 |
| 2 | rs2011559 | G | A | 0.1651 | 0.8349 | 7.79E−05 | 1.063487 |
| 2 | rs12694339 | A | G | 0.1384 | 0.8616 | 5.52E−04 | 1.045551 |
| 2 | rs17459063 | C | T | 0.2891 | 0.7109 | 1.19E−04 | 0.958479 |
| 2 | rs116109998 | T | C | 0.01771 | 0.98229 | 1.08E−04 | 1.210153 |
| 2 | rs1861630 | T | C | 0.1485 | 0.8515 | 7.13E−04 | 1.045686 |
| 2 | rs61741262 | C | T | 0.1163 | 0.8837 | 1.43E−04 | 0.931767 |
| 2 | rs2571442 | G | A | 0.4048 | 0.5952 | 1.72E−07 | 0.950427 |
| 2 | rs2943634 | A | C | 0.327 | 0.673 | 6.39E−05 | 0.959523 |
| 2 | rs116357755 | T | C | 0.03428 | 0.96572 | 1.08E−04 | 0.835474 |
| 2 | rs4500930 | T | C | 0.3421 | 0.6579 | 1.57E−04 | 1.038448 |
| 2 | rs7566501 | T | C | 0.4046 | 0.5954 | 3.69E−04 | 0.966564 |
| 2 | rs1801251 | A | G | 0.3647 | 0.6353 | 1.06E−04 | 1.039255 |
| 2 | rs115884546 | A | G | 0.02061 | 0.97939 | 9.73E−05 | 1.119576 |
| 2 | rs12052878 | A | G | 0.3149 | 0.6851 | 9.63E−04 | 0.966498 |
| 2 | rs62189896 | G | A | 0.1309 | 0.8691 | 4.67E−04 | 0.954085 |
| 2 | rs7596240 | G | A | 0.2702 | 0.7298 | 6.21E−04 | 1.041049 |
| 3 | rs11129351 | T | C | 0.4984 | 0.5016 | 9.29E−04 | 0.968818 |
| 3 | rs17723168 | G | A | 0.1809 | 0.8191 | 0.001225 | 1.044393 |
| 3 | rs112501139 | A | G | 0.0287 | 0.9713 | 3.74E−04 | 0.848465 |
| 3 | rs4684859 | A | G | 0.4246 | 0.5754 | 7.11E−04 | 1.033301 |
| 3 | rs294610 | A | C | 0.3423 | 0.6577 | 2.47E−04 | 1.035484 |
| 3 | rs2342881 | C | T | 0.1033 | 0.8967 | 1.58E−04 | 1.051905 |
| 3 | rs6806067 | A | C | 0.4214 | 0.5786 | 5.78E−05 | 1.037994 |
| 3 | rs79350839 | C | T | 0.008475 | 0.991525 | 8.70E−04 | 0.89636 |
| 3 | rs73078357 | C | T | 0.1197 | 0.8803 | 4.81E−06 | 0.935415 |
| 3 | rs78807522 | A | C | 0.1172 | 0.8828 | 1.91E−05 | 0.930526 |
| 3 | rs7623687 | C | A | 0.1404 | 0.8596 | 5.22E−07 | 0.932527 |
| 3 | rs11130199 | C | T | 0.4377 | 0.5623 | 8.73E−04 | 1.032328 |
| 3 | rs73079003 | A | G | 0.1245 | 0.8755 | 4.25E−06 | 0.927353 |
| 3 | rs9821675 | A | G | 0.4918 | 0.5082 | 2.63E−04 | 0.965295 |
| 3 | rs12053904 | T | C | 0.3376 | 0.6624 | 4.82E−05 | 0.960164 |
| 3 | rs9876685 | C | T | 0.01466 | 0.98534 | 9.68E−04 | 1.084003 |
| 3 | rs6792408 | A | G | 0.2556 | 0.7444 | 9.99E−04 | 1.035661 |
| 3 | rs2680259 | A | C | 0.448 | 0.552 | 5.30E−04 | 0.96761 |
| 3 | rs73134199 | C | T | 0.08519 | 0.91481 | 0.001117 | 1.06702 |
| 3 | rs58889931 | T | G | 0.4684 | 0.5316 | 3.57E−04 | 0.967561 |
| 3 | rs10937540 | T | C | 0.3197 | 0.6803 | 5.52E−04 | 1.033739 |
| 3 | rs116740640 | A | G | 0.02338 | 0.97662 | 6.78E−04 | 1.175438 |
| 3 | rs62264867 | C | T | 0.02011 | 0.97989 | 6.52E−04 | 1.125761 |
| 3 | rs4855890 | G | A | 0.4544 | 0.5456 | 4.39E−04 | 0.968027 |
| 3 | rs17307972 | T | G | 0.04375 | 0.95625 | 5.84E−04 | 1.107173 |
| 3 | rs6438855 | G | A | 0.1444 | 0.8556 | 1.12E−04 | 1.049637 |
| 3 | rs9839328 | C | T | 0.2413 | 0.7587 | 2.61E−04 | 1.040082 |
| 3 | rs114990251 | T | G | 0.01368 | 0.98632 | 0.001154 | 1.155154 |
| 3 | rs35782477 | G | T | 0.1416 | 0.8584 | 4.57E−04 | 1.058457 |
| 3 | rs9819988 | C | T | 0.3344 | 0.6656 | 1.21E−04 | 1.039193 |
| 3 | rs61789562 | C | T | 0.117 | 0.883 | 2.05E−05 | 0.934923 |
| 3 | rs7612160 | A | C | 0.3417 | 0.6583 | 6.57E−05 | 1.040453 |
| 3 | rs71630059 | A | G | 0.2097 | 0.7903 | 4.24E−04 | 0.959453 |
| 3 | rs2306374 | C | T | 0.1636 | 0.8364 | 1.43E−06 | 1.067769 |
| 3 | rs10935282 | A | G | 0.4527 | 0.5473 | 7.13E−04 | 0.968427 |
| 3 | rs115479708 | G | A | 0.06111 | 0.93889 | 9.33E−04 | 0.92072 |
| 3 | rs4441632 | T | C | 0.4881 | 0.5119 | 2.67E−04 | 0.967083 |
| 3 | rs112633398 | A | G | 0.02622 | 0.97378 | 2.50E−04 | 1.153068 |
| 3 | rs4603932 | T | G | 0.2687 | 0.7313 | 2.45E−04 | 1.038129 |
| 3 | rs13073025 | C | T | 0.2615 | 0.7385 | 3.26E−04 | 0.962998 |
| 3 | rs355770 | G | A | 0.1568 | 0.8432 | 2.29E−05 | 0.938882 |
| 3 | rs9837095 | A | G | 0.05812 | 0.94188 | 4.21E−04 | 0.931226 |
| 3 | rs114293154 | G | A | 0.02185 | 0.97815 | 0.001099 | 1.148286 |
| 3 | rs12486090 | C | A | 0.1208 | 0.8792 | 0.001088 | 1.050842 |
| 3 | rs12897 | G | A | 0.3662 | 0.6338 | 4.33E−07 | 1.050933 |
| 3 | rs1403050 | G | A | 0.2018 | 0.7982 | 6.17E−04 | 1.039137 |
| 3 | rs73206664 | G | A | 0.04325 | 0.95675 | 7.12E−04 | 1.146273 |
| 4 | rs73795019 | A | G | 0.0414 | 0.9586 | 5.97E−04 | 0.914368 |
| 4 | rs76613290 | T | C | 0.02801 | 0.97199 | 3.69E−04 | 0.854946 |
| 4 | rs76007570 | A | G | 0.1326 | 0.8674 | 7.72E−04 | 0.945593 |
| 4 | rs11938561 | C | A | 0.3278 | 0.6722 | 0.001007 | 0.966939 |
| 4 | rs16994919 | G | A | 0.07444 | 0.92556 | 5.42E−06 | 0.934837 |
| 4 | rs116697279 | A | G | 0.01009 | 0.98991 | 3.98E−04 | 1.194289 |
| 4 | rs66487928 | T | C | 0.139 | 0.861 | 7.74E−04 | 0.954423 |
| 4 | rs56321117 | G | T | 0.0749 | 0.9251 | 0.001207 | 1.053493 |
| 4 | rs1382083 | A | G | 0.209 | 0.791 | 1.58E−04 | 1.047313 |
| 4 | rs56155140 | A | G | 0.1858 | 0.8142 | 8.81E−08 | 1.061209 |
| 4 | rs6554520 | C | T | 0.1811 | 0.8189 | 9.54E−04 | 0.957046 |
| 4 | rs6845297 | G | A | 0.3976 | 0.6024 | 5.27E−04 | 0.963066 |
| 4 | rs1458038 | T | C | 0.2945 | 0.7055 | 6.28E−06 | 1.04717 |
| 4 | rs11939325 | C | T | 0.2032 | 0.7968 | 1.43E−04 | 0.953825 |
| 4 | rs71596078 | G | T | 0.02313 | 0.97687 | 2.56E−04 | 0.783096 |
| 4 | rs2868761 | G | A | 0.0304 | 0.9696 | 8.30E−04 | 1.095027 |
| 4 | rs4699312 | T | C | 0.271 | 0.729 | 9.36E−06 | 0.953962 |
| 4 | rs74694950 | C | A | 0.03877 | 0.96123 | 0.00114 | 1.088965 |
| 4 | rs3816873 | C | T | 0.2539 | 0.7461 | 2.90E−04 | 0.962792 |
| 4 | rs2522517 | G | A | 0.2109 | 0.7891 | 4.52E−04 | 1.040629 |
| 4 | rs58232303 | G | A | 0.006176 | 0.993824 | 8.52E−04 | 1.228598 |
| 4 | rs75973237 | C | T | 0.02677 | 0.97323 | 1.95E−04 | 1.180592 |
| 4 | rs13110872 | T | C | 0.2998 | 0.7002 | 4.76E−04 | 1.034339 |
| 4 | rs35922174 | C | T | 0.1058 | 0.8942 | 2.16E−04 | 0.93005 |
| 4 | rs11737246 | A | G | 0.02218 | 0.97782 | 9.79E−04 | 0.888255 |
| 4 | rs62319649 | C | T | 0.4058 | 0.5942 | 2.65E−04 | 1.038343 |
| 4 | rs11723436 | G | A | 0.3155 | 0.6845 | 1.68E−07 | 1.055495 |
| 4 | rs6853939 | C | T | 0.4467 | 0.5533 | 3.59E−04 | 1.039717 |
| 4 | rs34087892 | T | C | 0.1113 | 0.8887 | 1.22E−04 | 1.057444 |
| 4 | rs11728621 | G | A | 0.2087 | 0.7913 | 0.001217 | 0.960803 |
| 4 | rs10008867 | A | G | 0.3744 | 0.6256 | 8.04E−04 | 1.037028 |
| 4 | rs66596099 | T | C | 0.05297 | 0.94703 | 0.00118 | 1.059887 |
| 4 | rs10030732 | T | C | 0.3086 | 0.6914 | 1.55E−05 | 0.953993 |
| 4 | rs10857425 | A | G | 0.2682 | 0.7318 | 6.29E−04 | 1.038652 |
| 4 | rs13143223 | T | C | 0.06515 | 0.93485 | 1.70E−04 | 0.906875 |
| 4 | rs34712672 | A | G | 0.06865 | 0.93135 | 6.32E−05 | 0.91041 |
| 4 | rs7667633 | C | T | 0.1728 | 0.8272 | 5.64E−09 | 0.935521 |
| 4 | rs1878406 | T | C | 0.14 | 0.86 | 1.24E−06 | 1.06229 |
| 4 | rs4690974 | C | T | 0.4708 | 0.5292 | 2.63E−04 | 1.035149 |
| 4 | rs2880099 | A | C | 0.1049 | 0.8951 | 6.44E−06 | 1.059694 |
| 4 | rs1842896 | G | T | 0.4782 | 0.5218 | 5.82E−06 | 0.958623 |
| 4 | rs7658967 | A | G | 0.3483 | 0.6517 | 5.75E−04 | 0.963863 |
| 4 | rs3796585 | A | G | 0.3614 | 0.6386 | 4.98E−08 | 0.949582 |
| 4 | rs3796570 | A | C | 0.1395 | 0.8605 | 4.57E−04 | 0.955514 |
| 4 | rs17542750 | C | T | 0.4292 | 0.5708 | 0.001134 | 1.031578 |
| 4 | rs17627781 | G | A | 0.05893 | 0.94107 | 6.42E−04 | 0.910032 |
| 4 | rs4260573 | C | T | 0.4846 | 0.5154 | 3.85E−04 | 1.034613 |
| 4 | rs13104388 | T | C | 0.03963 | 0.96037 | 7.04E−05 | 0.870632 |
| 5 | rs2853676 | T | C | 0.2693 | 0.7307 | 0.001116 | 0.962129 |
| 5 | rs17636294 | A | G | 0.01818 | 0.98182 | 4.93E−04 | 0.861333 |
| 5 | rs11748327 | T | C | 0.3051 | 0.6949 | 2.12E−05 | 0.956205 |
| 5 | rs274723 | A | G | 0.329 | 0.671 | 0.001071 | 0.969048 |
| 5 | rs17328863 | T | C | 0.1407 | 0.8593 | 5.19E−05 | 0.941837 |
| 5 | rs150625 | A | C | 0.1289 | 0.8711 | 0.001155 | 1.044806 |
| 5 | rs906070 | T | C | 0.1246 | 0.8754 | 1.29E−04 | 1.052882 |
| 5 | rs2086606 | A | G | 0.3005 | 0.6995 | 0.001063 | 1.034586 |
| 5 | rs4379197 | G | A | 0.4365 | 0.5635 | 0.001091 | 0.968826 |
| 5 | rs4074793 | G | A | 0.07476 | 0.92524 | 9.87E−06 | 1.078061 |
| 5 | rs7727248 | C | T | 0.1589 | 0.8411 | 4.22E−04 | 1.041909 |
| 5 | rs2897686 | C | T | 0.1888 | 0.8112 | 1.71E−04 | 1.045381 |
| 5 | rs3936510 | T | G | 0.2014 | 0.7986 | 4.73E−04 | 1.043184 |
| 5 | rs112662197 | A | C | 0.02667 | 0.97333 | 6.07E−04 | 1.137919 |
| 5 | rs3756668 | A | G | 0.4582 | 0.5418 | 0.001112 | 0.969282 |
| 5 | rs12916 | C | T | 0.4 | 0.6 | 1.59E−04 | 1.036477 |
| 5 | rs7712660 | A | G | 0.418 | 0.582 | 5.29E−05 | 1.039513 |
| 5 | rs34228777 | T | C | 0.2381 | 0.7619 | 5.09E−04 | 0.963049 |
| 5 | rs12658882 | G | A | 0.1992 | 0.8008 | 6.77E−04 | 0.960957 |
| 5 | rs2916577 | G | A | 0.1539 | 0.8461 | 1.22E−05 | 0.947213 |
| 5 | rs77606373 | C | A | 0.08376 | 0.91624 | 2.66E−04 | 0.939775 |
| 5 | rs111756120 | T | C | 0.02468 | 0.97532 | 2.43E−04 | 0.884578 |
| 5 | rs2979862 | T | C | 0.1689 | 0.8311 | 2.48E−04 | 1.047862 |
| 5 | rs2303720 | T | C | 0.02273 | 0.97727 | 5.96E−04 | 0.928671 |
| 5 | rs4530754 | G | A | 0.4571 | 0.5429 | 1.90E−04 | 1.035886 |
| 5 | rs35003007 | T | C | 0.3238 | 0.6762 | 3.67E−04 | 0.958269 |
| 5 | rs78617572 | A | G | 0.02845 | 0.97155 | 7.92E−05 | 0.835504 |
| 5 | rs273909 | G | A | 0.1178 | 0.8822 | 1.24E−04 | 1.057508 |
| 5 | rs115502334 | A | G | 0.01555 | 0.98445 | 9.97E−04 | 1.232146 |
| 5 | rs11167756 | C | T | 0.4169 | 0.5831 | 1.79E−04 | 1.037323 |
| 5 | rs3776307 | G | A | 0.468 | 0.532 | 9.56E−05 | 1.040082 |
| 5 | rs7724577 | C | T | 0.2994 | 0.7006 | 1.34E−04 | 0.961716 |
| 5 | rs281053 | T | G | 0.1644 | 0.8356 | 1.31E−04 | 0.951529 |
| 5 | rs29784 | T | C | 0.4654 | 0.5346 | 0.00106 | 1.031983 |
| 6 | rs72836800 | C | T | 0.1332 | 0.8668 | 1.74E−04 | 0.942136 |
| 6 | rs2450303 | T | G | 0.4664 | 0.5336 | 0.001212 | 1.031027 |
| 6 | rs72819494 | G | T | 0.1432 | 0.8568 | 0.001208 | 0.950371 |
| 6 | rs4560685 | C | A | 0.351 | 0.649 | 2.21E−05 | 1.046387 |
| 6 | rs452658 | T | C | 0.3073 | 0.6927 | 0.001006 | 0.968174 |
| 6 | rs4713395 | G | T | 0.07888 | 0.92112 | 3.08E−04 | 0.932892 |
| 6 | rs75903935 | T | C | 0.04932 | 0.95068 | 1.45E−04 | 1.104455 |
| 6 | rs9463136 | C | T | 0.3445 | 0.6555 | 2.04E−07 | 0.948799 |
| 6 | rs9349379 | G | A | 0.405 | 0.595 | 1.81E−42 | 1.140921 |
| 6 | rs111969884 | T | G | 0.05867 | 0.94133 | 3.61E−05 | 1.117419 |
| 6 | rs76282554 | A | G | 0.03012 | 0.96988 | 6.62E−04 | 1.139662 |
| 6 | rs62389460 | A | G | 0.1247 | 0.8753 | 1.30E−07 | 0.916305 |
| 6 | rs7773701 | A | G | 0.3147 | 0.6853 | 6.63E−04 | 0.965218 |
| 6 | rs10484342 | C | T | 0.1265 | 0.8735 | 1.64E−04 | 0.950877 |
| 6 | rs113577849 | T | C | 0.03262 | 0.96738 | 3.17E−04 | 1.135383 |
| 6 | rs6900627 | A | G | 0.2878 | 0.7122 | 3.56E−04 | 1.040205 |
| 6 | rs12526553 | G | A | 0.03001 | 0.96999 | 0.001034 | 0.920086 |
| 6 | rs150533 | G | A | 0.1582 | 0.8418 | 3.67E−05 | 1.05225 |
| 6 | rs116186273 | C | T | 0.04613 | 0.95387 | 5.84E−04 | 1.1019 |
| 6 | rs2844670 | G | A | 0.1688 | 0.8312 | 0.001036 | 0.95279 |
| 6 | rs3906263 | A | G | 0.2171 | 0.7829 | 1.10E−06 | 1.064169 |
| 6 | rs9461684 | T | C | 0.08321 | 0.91679 | 1.48E−04 | 1.062352 |
| 6 | rs2442752 | C | T | 0.4254 | 0.5746 | 9.60E−07 | 1.053332 |
| 6 | rs2844519 | T | C | 0.2093 | 0.7907 | 6.07E−04 | 0.94882 |
| 6 | rs9267649 | A | G | 0.1898 | 0.8102 | 2.86E−04 | 0.950694 |
| 6 | rs149995364 | T | C | 0.00804 | 0.99196 | 4.58E−04 | 0.825026 |
| 6 | rs498422 | G | T | 0.05681 | 0.94319 | 9.53E−04 | 0.942746 |
| 6 | rs34451186 | C | T | 0.1194 | 0.8806 | 5.66E−04 | 1.096251 |
| 6 | rs9276826 | G | A | 0.1021 | 0.8979 | 7.29E−04 | 1.063007 |
| 6 | rs16894959 | C | T | 0.1407 | 0.8593 | 0.001098 | 1.046806 |
| 6 | rs114045003 | C | T | 0.00905 | 0.99095 | 6.62E−04 | 1.228751 |
| 6 | rs13203701 | G | A | 0.1692 | 0.8308 | 3.29E−04 | 0.949411 |
| 6 | rs4714074 | A | G | 0.1423 | 0.8577 | 0.001225 | 1.044237 |
| 6 | rs55856036 | C | A | 0.2158 | 0.7842 | 4.11E−08 | 0.937952 |
| 6 | rs10456100 | T | C | 0.2823 | 0.7177 | 1.75E−04 | 1.040296 |
| 6 | rs742493 | C | T | 0.1118 | 0.8882 | 1.14E−05 | 0.934976 |
| 6 | rs6905288 | G | A | 0.4326 | 0.5674 | 4.58E−05 | 0.959252 |
| 6 | rs12527048 | T | C | 0.03618 | 0.96382 | 0.001231 | 1.104575 |
| 6 | rs4610542 | T | G | 0.05001 | 0.94999 | 8.88E−04 | 0.921782 |
| 6 | rs1337351 | A | G | 0.3903 | 0.6097 | 6.52E−04 | 1.032441 |
| 6 | rs72906417 | A | G | 0.03636 | 0.96364 | 7.66E−04 | 0.89589 |
| 6 | rs13191092 | G | A | 0.03746 | 0.96254 | 2.41E−04 | 0.889513 |
| 6 | rs9359359 | T | C | 0.4667 | 0.5333 | 7.47E−04 | 0.969005 |
| 6 | rs194927 | G | A | 0.1566 | 0.8434 | 1.52E−05 | 1.054903 |
| 6 | rs1931647 | C | T | 0.2762 | 0.7238 | 5.67E−04 | 1.037455 |
| 6 | rs9361871 | G | T | 0.4866 | 0.5134 | 2.06E−04 | 0.965466 |
| 6 | rs117670960 | T | C | 0.02646 | 0.97354 | 0.001163 | 1.17511 |
| 6 | rs12207570 | G | T | 0.1897 | 0.8103 | 5.57E−06 | 0.946427 |
| 6 | rs11153674 | T | G | 0.1255 | 0.8745 | 2.50E−04 | 1.057023 |
| 6 | rs1339016 | C | T | 0.1278 | 0.8722 | 3.77E−04 | 0.949932 |
| 6 | rs12664359 | A | G | 0.2761 | 0.7239 | 5.68E−04 | 0.964641 |
| 6 | rs9388489 | G | A | 0.4498 | 0.5502 | 1.84E−04 | 0.964578 |
| 6 | rs4549631 | C | T | 0.4919 | 0.5081 | 0.001231 | 0.969416 |
| 6 | rs17459173 | C | A | 0.05264 | 0.94736 | 8.13E−04 | 1.105132 |
| 6 | rs76712796 | T | C | 0.05872 | 0.94128 | 0.001016 | 0.920058 |
| 6 | rs12524865 | A | C | 0.2895 | 0.7105 | 5.11E−11 | 0.936565 |
| 6 | rs162187 | T | C | 0.06949 | 0.93051 | 9.69E−06 | 1.078872 |
| 6 | rs2627234 | T | C | 0.4953 | 0.5047 | 8.82E−04 | 1.031814 |
| 6 | rs1345993 | T | G | 0.4093 | 0.5907 | 4.66E−04 | 1.033183 |
| 6 | rs74493285 | A | G | 0.03635 | 0.96365 | 3.94E−04 | 0.897943 |
| 6 | rs512685 | A | G | 0.1238 | 0.8762 | 3.62E−05 | 0.950113 |
| 6 | rs73613423 | G | T | 0.05001 | 0.94999 | 0.001029 | 1.08003 |
| 6 | rs62435969 | C | T | 0.1041 | 0.8959 | 5.21E−04 | 1.04891 |
| 6 | rs3798164 | A | G | 0.3887 | 0.6113 | 1.77E−04 | 0.965394 |
| 6 | rs624249 | A | C | 0.4012 | 0.5988 | 6.82E−09 | 0.940574 |
| 6 | rs3127573 | G | A | 0.1352 | 0.8648 | 4.38E−05 | 1.06075 |
| 6 | rs10080815 | G | T | 0.01988 | 0.98012 | 1.33E−15 | 1.279702 |
| 6 | rs3918291 | C | T | 0.008731 | 0.991269 | 3.71E−07 | 1.409056 |
| 6 | rs2292334 | A | G | 0.3923 | 0.6077 | 2.26E−09 | 1.059163 |
| 6 | rs6919346 | T | C | 0.1662 | 0.8338 | 2.37E−04 | 0.949966 |
| 6 | rs3798220 | C | T | 0.01847 | 0.98153 | 4.66E−09 | 1.419554 |
| 6 | rs77009508 | G | A | 0.07516 | 0.92484 | 4.04E−05 | 0.9081 |
| 6 | rs10455872 | G | A | 0.08115 | 0.91885 | 5.73E−39 | 1.375198 |
| 6 | rs41272044 | C | T | 0.191 | 0.809 | 2.87E−07 | 0.944546 |
| 6 | rs73596816 | A | G | 0.03321 | 0.96679 | 1.68E−04 | 1.136244 |
| 6 | rs6935921 | C | T | 0.2998 | 0.7002 | 7.35E−10 | 0.940939 |
| 6 | rs9457998 | G | A | 0.1632 | 0.8368 | 1.63E−04 | 1.051982 |
| 6 | rs783147 | A | G | 0.4441 | 0.5559 | 3.14E−05 | 0.962258 |
| 6 | rs12204899 | G | A | 0.09942 | 0.90058 | 3.29E−04 | 1.075555 |
| 6 | rs55754459 | G | A | 0.02909 | 0.97091 | 3.31E−05 | 1.136215 |
| 6 | rs7761309 | G | A | 0.4407 | 0.5593 | 5.43E−05 | 1.040424 |
| 6 | rs396207 | C | T | 0.2903 | 0.7097 | 9.94E−04 | 1.032958 |
| 7 | rs111812602 | A | G | 0.04986 | 0.95014 | 6.57E−04 | 0.902356 |
| 7 | rs6464022 | T | C | 0.3365 | 0.6635 | 7.52E−05 | 0.95799 |
| 7 | rs10247428 | A | G | 0.4125 | 0.5875 | 4.67E−04 | 1.03509 |
| 7 | rs117925813 | T | C | 0.02061 | 0.97939 | 0.001015 | 0.839075 |
| 7 | rs76664514 | T | C | 0.04322 | 0.95678 | 2.45E−04 | 1.086742 |
| 7 | rs11984041 | T | C | 0.09652 | 0.90348 | 5.07E−05 | 1.066005 |
| 7 | rs12530920 | C | A | 0.07764 | 0.92236 | 2.66E−06 | 1.067544 |
| 7 | rs11763270 | A | C | 0.257 | 0.743 | 6.93E−06 | 1.047047 |
| 7 | rs34889455 | A | C | 0.2423 | 0.7577 | 7.19E−04 | 0.95635 |
| 7 | rs73688135 | C | T | 0.04278 | 0.95722 | 0.001072 | 0.935996 |
| 7 | rs35959088 | A | G | 0.4018 | 0.5982 | 7.48E−04 | 1.032606 |
| 7 | rs9701 | G | A | 0.09794 | 0.90206 | 6.29E−04 | 1.045369 |
| 7 | rs73121539 | T | G | 0.03407 | 0.96593 | 5.02E−04 | 0.871132 |
| 7 | rs4148810 | T | C | 0.1795 | 0.8205 | 7.60E−04 | 0.962006 |
| 7 | rs2572023 | A | G | 0.4746 | 0.5254 | 4.52E−04 | 0.967694 |
| 7 | rs2070215 | C | T | 0.286 | 0.714 | 3.22E−04 | 1.038896 |
| 7 | rs7778388 | G | A | 0.3822 | 0.6178 | 9.20E−04 | 1.033676 |
| 7 | rs62483669 | G | A | 0.09927 | 0.90073 | 8.84E−04 | 1.066796 |
| 7 | rs10216100 | C | A | 0.05135 | 0.94865 | 7.08E−04 | 1.072462 |
| 7 | rs73184021 | A | G | 0.04307 | 0.95693 | 3.86E−05 | 1.144905 |
| 7 | rs1002935 | A | G | 0.2263 | 0.7737 | 2.51E−04 | 0.958884 |
| 7 | rs2520249 | G | A | 0.2887 | 0.7113 | 1.14E−04 | 1.040827 |
| 7 | rs10953541 | T | C | 0.2519 | 0.7481 | 1.02E−05 | 0.950915 |
| 7 | rs1405278 | A | G | 0.005711 | 0.994289 | 2.30E−04 | 0.809771 |
| 7 | rs117333120 | T | C | 0.02997 | 0.97003 | 2.46E−04 | 1.187202 |
| 7 | rs11556924 | T | C | 0.3899 | 0.6101 | 5.34E−11 | 0.930002 |
| 7 | rs57450990 | A | G | 0.04085 | 0.95915 | 7.07E−04 | 0.924118 |
| 7 | rs73160719 | C | T | 0.06811 | 0.93189 | 8.16E−04 | 0.938558 |
| 7 | rs28707561 | T | C | 0.1842 | 0.8158 | 7.70E−04 | 0.955572 |
| 7 | rs2286195 | C | A | 0.1974 | 0.8026 | 2.05E−05 | 0.953536 |
| 7 | rs118102967 | G | T | 0.02443 | 0.97557 | 2.31E−04 | 0.896495 |
| 7 | rs117564322 | A | G | 0.03228 | 0.96772 | 2.29E−04 | 1.162439 |
| 8 | rs17066350 | A | G | 0.0275 | 0.9725 | 6.86E−04 | 0.891598 |
| 8 | rs73508127 | A | G | 0.05933 | 0.94067 | 1.27E−04 | 1.091077 |
| 8 | rs10109286 | C | T | 0.1824 | 0.8176 | 4.42E−04 | 1.042679 |
| 8 | rs1441778 | C | T | 0.1551 | 0.8449 | 4.74E−04 | 0.952031 |
| 8 | rs264 | A | G | 0.1401 | 0.8599 | 1.06E−05 | 0.943362 |
| 8 | rs2083637 | G | A | 0.2638 | 0.7362 | 1.93E−06 | 0.94938 |
| 8 | rs4557718 | C | T | 0.1339 | 0.8661 | 8.71E−04 | 1.061108 |
| 8 | rs73225862 | T | C | 0.05578 | 0.94422 | 2.38E−04 | 1.095582 |
| 8 | rs2704067 | C | T | 0.4337 | 0.5663 | 7.48E−04 | 1.032966 |
| 8 | rs34220779 | C | A | 0.1146 | 0.8854 | 5.56E−04 | 1.060629 |
| 8 | rs55642871 | C | T | 0.03453 | 0.96547 | 9.94E−04 | 0.907136 |
| 8 | rs35089546 | C | T | 0.009375 | 0.990625 | 8.31E−04 | 1.129974 |
| 8 | rs72639778 | T | C | 0.01944 | 0.98056 | 7.98E−05 | 1.215908 |
| 8 | rs74744524 | T | C | 0.01827 | 0.98173 | 8.75E−04 | 0.893842 |
| 8 | rs7841815 | A | G | 0.4639 | 0.5361 | 9.42E−04 | 0.969804 |
| 8 | rs116932954 | C | T | 0.01463 | 0.98537 | 2.46E−04 | 1.202758 |
| 8 | rs34489694 | T | C | 0.1766 | 0.8234 | 6.07E−04 | 1.043556 |
| 8 | rs10504385 | G | T | 0.2699 | 0.7301 | 7.41E−04 | 0.966486 |
| 8 | rs2933045 | T | C | 0.3727 | 0.6273 | 2.88E−04 | 0.966186 |
| 8 | rs7842297 | C | T | 0.217 | 0.783 | 7.93E−04 | 1.038889 |
| 8 | rs75671747 | C | T | 0.04972 | 0.95028 | 8.74E−04 | 1.074865 |
| 8 | rs72672639 | A | C | 0.1372 | 0.8628 | 3.58E−05 | 1.057245 |
| 8 | rs2010013 | G | T | 0.3088 | 0.6912 | 7.84E−05 | 1.040008 |
| 8 | rs17336058 | T | C | 0.1938 | 0.8062 | 2.93E−05 | 1.051055 |
| 8 | rs77211063 | T | C | 0.03461 | 0.96539 | 1.06E−04 | 1.134505 |
| 8 | rs13257909 | G | A | 0.3131 | 0.6869 | 0.001038 | 0.967944 |
| 8 | rs72671684 | A | G | 0.02617 | 0.97383 | 0.001145 | 0.844942 |
| 8 | rs10103119 | A | G | 0.4866 | 0.5134 | 5.10E−04 | 0.968479 |
| 8 | rs7350129 | T | G | 0.2914 | 0.7086 | 0.001079 | 0.967774 |
| 8 | rs10956199 | T | C | 0.2881 | 0.7119 | 8.08E−04 | 1.040124 |
| 8 | rs6982502 | C | T | 0.4785 | 0.5215 | 8.85E−07 | 1.046384 |
| 8 | rs117012718 | G | A | 0.01854 | 0.98146 | 9.43E−04 | 0.812378 |
| 8 | rs622856 | T | C | 0.3654 | 0.6346 | 5.75E−04 | 0.966174 |
| 8 | rs12155935 | T | C | 0.08085 | 0.91915 | 5.44E−04 | 1.049176 |
| 8 | rs1411187 | A | G | 0.4904 | 0.5096 | 7.00E−04 | 1.032204 |
| 8 | rs74919147 | G | T | 0.04608 | 0.95392 | 8.13E−04 | 0.904731 |
| 8 | rs753778 | A | G | 0.296 | 0.704 | 2.72E−05 | 0.958693 |
| 8 | rs55846720 | G | A | 0.4331 | 0.5669 | 8.63E−04 | 0.968284 |
| 8 | rs10103713 | A | G | 0.03332 | 0.96668 | 9.45E−04 | 1.120204 |
| 8 | rs4961383 | A | G | 0.4845 | 0.5155 | 3.85E−04 | 0.962109 |
| 8 | rs75548654 | G | A | 0.114 | 0.886 | 9.87E−04 | 1.053776 |
| 9 | rs79442118 | A | G | 0.01398 | 0.98602 | 4.75E−04 | 0.921954 |
| 9 | rs10814856 | C | T | 0.301 | 0.699 | 5.45E−04 | 1.035332 |
| 9 | rs1571225 | C | T | 0.1366 | 0.8634 | 0.001156 | 0.962245 |
| 9 | rs78350788 | T | G | 0.03731 | 0.96269 | 0.00103 | 1.105547 |
| 9 | rs79549765 | A | C | 0.09296 | 0.90704 | 6.36E−04 | 1.067506 |
| 9 | rs10114824 | C | A | 0.3324 | 0.6676 | 4.30E−04 | 0.964761 |
| 9 | rs10811104 | A | C | 0.4227 | 0.5773 | 8.25E−04 | 1.032132 |
| 9 | rs10965110 | A | G | 0.02921 | 0.97079 | 6.17E−04 | 1.125021 |
| 9 | rs10965153 | C | T | 0.4561 | 0.5439 | 2.34E−05 | 0.959762 |
| 9 | rs3731249 | T | C | 0.02678 | 0.97322 | 1.44E−08 | 1.186536 |
| 9 | rs117626887 | G | A | 0.02213 | 0.97787 | 5.26E−06 | 0.852811 |
| 9 | rs62556547 | C | T | 0.1311 | 0.8689 | 2.79E−14 | 0.880236 |
| 9 | rs4977754 | C | A | 0.04559 | 0.95441 | 5.92E−08 | 0.888991 |
| 9 | rs4977574 | G | A | 0.4868 | 0.5132 | 6.35E−98 | 1.212803 |
| 9 | rs7045889 | G | A | 0.4217 | 0.5783 | 1.18E−06 | 0.953508 |
| 9 | rs10780701 | A | G | 0.2827 | 0.7173 | 8.98E−05 | 1.04083 |
| 9 | rs2791568 | G | A | 0.066 | 0.934 | 3.12E−04 | 1.082341 |
| 9 | rs3933798 | T | C | 0.1852 | 0.8148 | 0.00101 | 0.964935 |
| 9 | rs2196797 | T | G | 0.01688 | 0.98312 | 5.42E−04 | 0.9028 |
| 9 | rs4149311 | T | C | 0.1189 | 0.8811 | 8.29E−05 | 1.052893 |
| 9 | rs10978182 | G | A | 0.1931 | 0.8069 | 0.001023 | 0.963074 |
| 9 | rs869374 | T | C | 0.1637 | 0.8363 | 8.55E−04 | 1.043496 |
| 9 | rs1467311 | G | A | 0.3369 | 0.6631 | 2.22E−05 | 1.042998 |
| 9 | rs10817276 | A | G | 0.3119 | 0.6881 | 2.96E−05 | 1.041492 |
| 9 | rs74982099 | T | C | 0.0768 | 0.9232 | 1.46E−04 | 1.068052 |
| 9 | rs793513 | G | A | 0.3236 | 0.6764 | 1.52E−04 | 0.963205 |
| 9 | rs7025486 | A | G | 0.2595 | 0.7405 | 3.26E−06 | 1.050446 |
| 9 | rs10739663 | G | A | 0.452 | 0.548 | 3.50E−04 | 1.033694 |
| 9 | rs953226 | A | C | 0.08679 | 0.91321 | 8.56E−04 | 0.937396 |
| 9 | rs2417157 | C | T | 0.4694 | 0.5306 | 8.10E−04 | 0.963802 |
| 9 | rs7868232 | C | T | 0.241 | 0.759 | 1.39E−04 | 1.048948 |
| 9 | rs507666 | A | G | 0.1845 | 0.8155 | 1.64E−11 | 1.081978 |
| 9 | rs73553519 | T | C | 0.09107 | 0.90893 | 1.40E−04 | 1.089609 |
| 9 | rs62589661 | A | G | 0.06137 | 0.93863 | 3.28E−04 | 0.880875 |
| 10 | rs17156036 | C | T | 0.2537 | 0.7463 | 7.23E−04 | 0.963795 |
| 10 | rs12359173 | G | A | 0.1337 | 0.8663 | 1.75E−04 | 0.912647 |
| 10 | rs7912831 | C | T | 0.483 | 0.517 | 7.01E−04 | 0.969241 |
| 10 | rs72777089 | A | G | 0.03901 | 0.96099 | 2.22E−04 | 0.876069 |
| 10 | rs11257613 | G | A | 0.4868 | 0.5132 | 0.001187 | 1.030188 |
| 10 | rs723210 | C | A | 0.4838 | 0.5162 | 4.08E−04 | 0.968209 |
| 10 | rs11596618 | A | G | 0.1419 | 0.8581 | 6.79E−04 | 1.04998 |
| 10 | rs11258278 | C | T | 0.03071 | 0.96929 | 7.94E−04 | 1.096999 |
| 10 | rs2505083 | C | T | 0.4315 | 0.5685 | 1.57E−10 | 1.062933 |
| 10 | rs471142 | T | C | 0.3355 | 0.6645 | 4.49E−04 | 0.965955 |
| 10 | rs72807309 | T | C | 0.01969 | 0.98031 | 7.55E−05 | 1.177442 |
| 10 | rs2284948 | G | T | 0.2278 | 0.7722 | 4.43E−04 | 1.036859 |
| 10 | rs1254849 | C | T | 0.07577 | 0.92423 | 7.04E−04 | 0.932192 |
| 10 | rs7067919 | A | G | 0.08676 | 0.91324 | 5.75E−04 | 0.946306 |
| 10 | rs2047009 | T | G | 0.4781 | 0.5219 | 2.75E−11 | 0.940581 |
| 10 | rs11238850 | G | A | 0.0773 | 0.9227 | 7.62E−04 | 0.943235 |
| 10 | rs680091 | A | G | 0.1027 | 0.8973 | 3.75E−12 | 0.908125 |
| 10 | rs2437935 | G | A | 0.3544 | 0.6456 | 6.98E−09 | 0.947001 |
| 10 | rs1370158 | T | C | 0.1643 | 0.8357 | 9.20E−04 | 0.959642 |
| 10 | rs1147885 | T | C | 0.2691 | 0.7309 | 1.10E−04 | 1.047384 |
| 10 | rs7078374 | G | A | 0.1214 | 0.8786 | 0.0011 | 0.953058 |
| 10 | rs7903630 | A | C | 0.01195 | 0.98805 | 0.001064 | 1.101592 |
| 10 | rs7901016 | C | T | 0.04901 | 0.95099 | 4.55E−04 | 0.944636 |
| 10 | rs72812300 | T | C | 0.1254 | 0.8746 | 9.56E−04 | 0.913379 |
| 10 | rs3911887 | A | G | 0.2827 | 0.7173 | 2.13E−04 | 0.96327 |
| 10 | rs11000909 | G | A | 0.1295 | 0.8705 | 0.001083 | 1.04607 |
| 10 | rs10740441 | G | A | 0.1541 | 0.8459 | 5.73E−04 | 1.04569 |
| 10 | rs12359810 | C | A | 0.07343 | 0.92657 | 0.001208 | 1.072936 |
| 10 | rs11001437 | G | A | 0.03314 | 0.96686 | 2.04E−04 | 1.152697 |
| 10 | rs11816174 | G | A | 0.2218 | 0.7782 | 7.85E−04 | 1.035918 |
| 10 | rs4934169 | A | G | 0.2723 | 0.7277 | 7.92E−06 | 1.052122 |
| 10 | rs35414786 | T | C | 0.2055 | 0.7945 | 6.13E−05 | 0.955346 |
| 10 | rs6586155 | T | C | 0.1071 | 0.8929 | 5.23E−04 | 0.957559 |
| 10 | rs55750333 | A | C | 0.3945 | 0.6055 | 6.03E−04 | 1.036818 |
| 10 | rs2071510 | T | G | 0.4027 | 0.5973 | 5.98E−04 | 1.033521 |
| 10 | rs6586178 | C | T | 0.03844 | 0.96156 | 9.12E−04 | 1.092663 |
| 10 | rs1412444 | T | C | 0.3382 | 0.6618 | 5.15E−12 | 1.069094 |
| 10 | rs11187165 | C | T | 0.2122 | 0.7878 | 2.76E−04 | 1.045057 |
| 10 | rs2068888 | A | G | 0.4489 | 0.5511 | 5.43E−05 | 0.96166 |
| 10 | rs6583943 | A | G | 0.4415 | 0.5585 | 5.86E−04 | 1.032665 |
| 10 | rs1983864 | G | T | 0.3383 | 0.6617 | 9.10E−04 | 0.968577 |
| 10 | rs7906144 | C | A | 0.3842 | 0.6158 | 7.06E−05 | 1.040482 |
| 10 | rs603424 | A | G | 0.1686 | 0.8314 | 3.76E−04 | 1.041006 |
| 10 | rs2031604 | T | C | 0.07866 | 0.92134 | 3.18E−04 | 0.952527 |
| 10 | rs2902548 | T | C | 0.1543 | 0.8457 | 9.24E−04 | 0.962174 |
| 10 | rs1004467 | G | A | 0.08358 | 0.91642 | 5.66E−09 | 0.925567 |
| 10 | rs743572 | G | A | 0.373 | 0.627 | 5.51E−06 | 0.958453 |
| 10 | rs11191548 | C | T | 0.07478 | 0.92522 | 5.66E−08 | 0.925782 |
| 10 | rs7896547 | G | A | 0.3871 | 0.6129 | 3.11E−04 | 0.967187 |
| 10 | rs76231175 | C | T | 0.01293 | 0.98707 | 1.42E−04 | 1.246493 |
| 10 | rs4751648 | C | T | 0.4533 | 0.5467 | 3.67E−04 | 1.034194 |
| 10 | rs61865314 | C | T | 0.2629 | 0.7371 | 1.65E−04 | 1.043063 |
| 10 | rs11199805 | T | C | 0.02131 | 0.97869 | 6.44E−04 | 0.927948 |
| 10 | rs74672221 | A | G | 0.07132 | 0.92868 | 3.06E−04 | 1.067726 |
| 10 | rs4752700 | G | A | 0.4285 | 0.5715 | 9.07E−04 | 1.031532 |
| 10 | rs7085665 | G | T | 0.1597 | 0.8403 | 8.25E−04 | 1.044246 |
| 10 | rs118063153 | T | C | 0.0194 | 0.9806 | 1.75E−04 | 0.861733 |
| 10 | rs11017716 | T | C | 0.09325 | 0.90675 | 6.57E−04 | 0.942839 |
| 11 | rs11038628 | T | C | 0.0574 | 0.9426 | 3.51E−04 | 0.945179 |
| 11 | rs2723641 | C | A | 0.2826 | 0.7174 | 2.07E−04 | 0.958772 |
| 11 | rs7395330 | C | T | 0.1013 | 0.8987 | 2.37E−05 | 1.058311 |
| 11 | rs4537761 | C | T | 0.0744 | 0.9256 | 6.68E−06 | 1.069514 |
| 11 | rs79149463 | G | T | 0.07985 | 0.92015 | 1.46E−04 | 1.084918 |
| 11 | rs2649048 | T | C | 0.3461 | 0.6539 | 7.10E−04 | 1.034165 |
| 11 | rs93138 | T | G | 0.4329 | 0.5671 | 5.54E−08 | 0.950247 |
| 11 | rs61876936 | C | T | 0.06427 | 0.93573 | 9.48E−04 | 0.91471 |
| 11 | rs900145 | C | T | 0.2931 | 0.7069 | 2.53E−06 | 0.954307 |
| 11 | rs35708656 | A | G | 0.17 | 0.83 | 0.001021 | 0.959641 |
| 11 | rs75313703 | G | T | 0.02267 | 0.97733 | 4.73E−04 | 0.867878 |
| 11 | rs16928390 | C | T | 0.2372 | 0.7628 | 6.31E−04 | 0.96255 |
| 11 | rs12292056 | A | C | 0.06637 | 0.93363 | 7.73E−04 | 0.936288 |
| 11 | rs72902692 | T | C | 0.06951 | 0.93049 | 0.00116 | 0.936103 |
| 11 | rs72908309 | T | C | 0.09607 | 0.90393 | 0.001041 | 0.943562 |
| 11 | rs35566076 | C | T | 0.03462 | 0.96538 | 0.001214 | 0.892899 |
| 11 | rs12801636 | A | G | 0.2242 | 0.7758 | 5.21E−06 | 0.951296 |
| 11 | rs11604462 | A | G | 0.3434 | 0.6566 | 1.43E−04 | 1.037874 |
| 11 | rs72991248 | T | C | 0.02007 | 0.97993 | 0.00105 | 0.855726 |
| 11 | rs606452 | A | C | 0.1401 | 0.8599 | 1.45E−04 | 0.952903 |
| 11 | rs2602488 | G | A | 0.3432 | 0.6568 | 6.92E−04 | 1.034385 |
| 11 | rs74812099 | G | A | 0.02663 | 0.97337 | 9.95E−04 | 0.883878 |
| 11 | rs17230594 | C | T | 0.02989 | 0.97011 | 8.16E−04 | 1.121402 |
| 11 | rs61908736 | G | A | 0.2705 | 0.7295 | 3.86E−05 | 1.048538 |
| 11 | rs17573732 | C | A | 0.03734 | 0.96266 | 7.89E−04 | 1.103929 |
| 11 | rs12788925 | G | A | 0.4482 | 0.5518 | 4.69E−07 | 0.953174 |
| 11 | rs1917467 | A | G | 0.1093 | 0.8907 | 0.00118 | 1.051786 |
| 11 | rs79917023 | G | A | 0.1162 | 0.8838 | 9.50E−04 | 0.950672 |
| 11 | rs1316710 | G | T | 0.1182 | 0.8818 | 5.71E−05 | 1.054404 |
| 11 | rs17318997 | C | T | 0.4554 | 0.5446 | 8.76E−04 | 1.031503 |
| 11 | rs7106274 | T | C | 0.1211 | 0.8789 | 5.67E−05 | 0.946483 |
| 11 | rs4938792 | C | T | 0.3899 | 0.6101 | 7.54E−04 | 1.03404 |
| 11 | rs114594921 | C | T | 0.01871 | 0.98129 | 1.83E−04 | 1.119475 |
| 11 | rs11820589 | A | G | 0.06633 | 0.93367 | 2.67E−04 | 1.073297 |
| 11 | rs662799 | G | A | 0.05894 | 0.94106 | 4.23E−04 | 1.05525 |
| 11 | rs79489590 | T | C | 0.03234 | 0.96766 | 0.001141 | 1.120425 |
| 11 | rs573455 | A | G | 0.4655 | 0.5345 | 7.20E−04 | 1.032425 |
| 11 | rs1077952 | A | C | 0.1076 | 0.8924 | 2.00E−04 | 1.050514 |
| 11 | rs1060542 | A | G | 0.05285 | 0.94715 | 8.90E−04 | 1.068822 |
| 12 | rs740769 | T | C | 0.4737 | 0.5263 | 8.32E−04 | 0.969434 |
| 12 | rs12146727 | A | G | 0.133 | 0.867 | 2.97E−04 | 0.950185 |
| 12 | rs2909001 | T | C | 0.1752 | 0.8248 | 5.18E−04 | 1.043393 |
| 12 | rs2417414 | G | A | 0.1566 | 0.8434 | 8.77E−04 | 0.954616 |
| 12 | rs4762746 | T | C | 0.2876 | 0.7124 | 6.88E−06 | 0.954933 |
| 12 | rs10770612 | G | A | 0.2 | 0.8 | 1.56E−04 | 1.044048 |
| 12 | rs12827742 | G | A | 0.2345 | 0.7655 | 4.21E−05 | 1.046635 |
| 12 | rs7136158 | A | G | 0.1758 | 0.8242 | 2.82E−04 | 1.041329 |
| 12 | rs75274818 | G | A | 0.06685 | 0.93315 | 6.14E−05 | 0.918588 |
| 12 | rs34697596 | G | A | 0.0637 | 0.9363 | 0.001138 | 1.121606 |
| 12 | rs11172113 | C | T | 0.4132 | 0.5868 | 1.71E−05 | 1.042253 |
| 12 | rs10083102 | C | T | 0.3099 | 0.6901 | 2.76E−04 | 0.963533 |
| 12 | rs75978279 | G | A | 0.0392 | 0.9608 | 1.90E−04 | 0.885217 |
| 12 | rs34441304 | C | A | 0.1539 | 0.8461 | 7.37E−05 | 0.943965 |
| 12 | rs1533514 | C | T | 0.2085 | 0.7915 | 7.81E−05 | 1.045521 |
| 12 | rs771628 | T | G | 0.3205 | 0.6795 | 0.001166 | 0.969178 |
| 12 | rs4842266 | G | A | 0.3047 | 0.6953 | 3.77E−04 | 0.966043 |
| 12 | rs872455 | C | T | 0.01366 | 0.98634 | 8.71E−04 | 0.936703 |
| 12 | rs2681472 | G | A | 0.1708 | 0.8292 | 6.17E−11 | 1.07693 |
| 12 | rs75138673 | G | T | 0.1509 | 0.8491 | 6.97E−04 | 0.951932 |
| 12 | rs117788490 | G | A | 0.03212 | 0.96788 | 6.20E−04 | 1.152961 |
| 12 | rs11107845 | A | G | 0.07735 | 0.92265 | 5.72E−04 | 0.943873 |
| 12 | rs6490029 | A | G | 0.2197 | 0.7803 | 4.50E−04 | 0.963756 |
| 12 | rs10774625 | A | G | 0.4958 | 0.5042 | 2.69E−10 | 1.068821 |
| 12 | rs4766897 | T | C | 0.3396 | 0.6604 | 1.20E−06 | 1.063647 |
| 12 | rs4646777 | A | G | 0.1711 | 0.8289 | 5.19E−05 | 0.953318 |
| 12 | rs17696736 | G | A | 0.4276 | 0.5724 | 7.06E−09 | 1.061987 |
| 12 | rs7965087 | A | G | 0.0943 | 0.9057 | 3.90E−04 | 0.950173 |
| 12 | rs11066301 | G | A | 0.4245 | 0.5755 | 2.06E−07 | 1.05876 |
| 12 | rs58116261 | T | C | 0.08048 | 0.91952 | 1.25E−04 | 0.940427 |
| 12 | rs10492024 | G | A | 0.38 | 0.62 | 1.46E−04 | 0.964305 |
| 12 | rs80116432 | C | T | 0.02371 | 0.97629 | 5.06E−04 | 1.14543 |
| 12 | rs7980629 | A | G | 0.06505 | 0.93495 | 3.17E−04 | 0.948093 |
| 12 | rs11067246 | G | A | 0.3619 | 0.6381 | 8.59E−04 | 1.034276 |
| 12 | rs2244608 | G | A | 0.3158 | 0.6842 | 1.02E−06 | 1.048703 |
| 12 | rs71456795 | C | T | 0.02616 | 0.97384 | 5.88E−04 | 0.846715 |
| 12 | rs12146870 | A | C | 0.1218 | 0.8782 | 3.30E−04 | 1.048678 |
| 13 | rs4770730 | C | T | 0.4488 | 0.5512 | 0.00105 | 1.032514 |
| 13 | rs3751395 | A | C | 0.4612 | 0.5388 | 4.50E−06 | 0.95363 |
| 13 | rs9508032 | C | T | 0.2538 | 0.7462 | 1.86E−04 | 0.961636 |
| 13 | rs748253 | T | G | 0.3553 | 0.6447 | 8.22E−04 | 1.033012 |
| 13 | rs7329021 | G | A | 0.4927 | 0.5073 | 9.43E−04 | 1.03171 |
| 13 | rs1887752 | T | C | 0.2607 | 0.7393 | 9.66E−04 | 1.033944 |
| 13 | rs117579501 | G | A | 0.05194 | 0.94806 | 8.38E−04 | 0.900921 |
| 13 | rs7998440 | A | G | 0.3652 | 0.6348 | 1.59E−05 | 0.957446 |
| 13 | rs7983267 | T | C | 0.4509 | 0.5491 | 3.92E−04 | 0.966599 |
| 13 | rs34525095 | T | C | 0.02805 | 0.97195 | 6.90E−04 | 0.874402 |
| 13 | rs11620516 | A | G | 0.37 | 0.63 | 1.43E−04 | 1.037159 |
| 13 | rs2589312 | A | G | 0.4199 | 0.5801 | 8.84E−04 | 0.967659 |
| 13 | rs80154967 | A | G | 0.007977 | 0.992023 | 9.43E−04 | 1.309164 |
| 13 | rs9316453 | A | C | 0.2933 | 0.7067 | 4.21E−04 | 1.037453 |
| 13 | rs17053844 | T | C | 0.05426 | 0.94574 | 1.10E−04 | 0.932892 |
| 13 | rs17789383 | G | T | 0.112 | 0.888 | 8.02E−04 | 0.950969 |
| 13 | rs17061555 | A | G | 0.2234 | 0.7766 | 0.001018 | 1.036004 |
| 13 | rs9573991 | A | C | 0.01811 | 0.98189 | 7.02E−04 | 0.922255 |
| 13 | rs1999018 | G | A | 0.0196 | 0.9804 | 0.001073 | 0.922141 |
| 13 | rs33949518 | T | C | 0.1561 | 0.8439 | 4.47E−04 | 0.953889 |
| 13 | rs17785077 | A | G | 0.1024 | 0.8976 | 7.16E−04 | 0.949127 |
| 13 | rs678994 | A | G | 0.4141 | 0.5859 | 6.73E−04 | 0.968994 |
| 13 | rs66895129 | G | A | 0.2706 | 0.7294 | 0.001085 | 0.966476 |
| 13 | rs12875271 | G | A | 0.0917 | 0.9083 | 2.89E−04 | 0.93923 |
| 13 | rs12428089 | C | T | 0.1585 | 0.8415 | 5.34E−04 | 0.957022 |
| 13 | rs3742207 | G | T | 0.3418 | 0.6582 | 3.91E−04 | 1.0354 |
| 13 | rs9521632 | C | T | 0.3701 | 0.6299 | 3.95E−05 | 1.040859 |
| 13 | rs4145073 | G | T | 0.3231 | 0.6769 | 2.67E−04 | 1.040684 |
| 13 | rs12873154 | G | A | 0.1352 | 0.8648 | 1.57E−05 | 1.067745 |
| 13 | rs4773144 | G | A | 0.4339 | 0.5661 | 3.87E−07 | 1.053781 |
| 13 | rs7334306 | T | C | 0.1632 | 0.8368 | 2.68E−06 | 1.091622 |
| 13 | rs35488904 | G | A | 0.2096 | 0.7904 | 1.74E−04 | 0.942334 |
| 13 | rs12867262 | G | A | 0.3422 | 0.6578 | 1.52E−05 | 1.050417 |
| 13 | rs9515203 | C | T | 0.264 | 0.736 | 9.33E−10 | 0.931326 |
| 13 | rs35166119 | A | C | 0.05153 | 0.94847 | 1.14E−04 | 1.093619 |
| 13 | rs35926204 | C | T | 0.1008 | 0.8992 | 3.65E−05 | 1.068277 |
| 13 | rs117412802 | G | A | 0.01399 | 0.98601 | 2.93E−04 | 1.193679 |
| 13 | rs9525238 | A | G | 0.1966 | 0.8034 | 1.17E−04 | 0.946704 |
| 14 | rs17122776 | G | A | 0.2298 | 0.7702 | 6.34E−04 | 1.045383 |
| 14 | rs1958060 | C | T | 0.105 | 0.895 | 3.36E−04 | 0.946461 |
| 14 | rs17102274 | G | A | 0.07131 | 0.92869 | 6.63E−04 | 1.053559 |
| 14 | rs72669646 | A | G | 0.01681 | 0.98319 | 9.24E−04 | 1.186753 |
| 14 | rs2084347 | T | C | 0.2851 | 0.7149 | 4.10E−04 | 0.962968 |
| 14 | rs7151210 | T | C | 0.161 | 0.839 | 5.97E−05 | 1.053081 |
| 14 | rs1204995 | T | C | 0.4477 | 0.5523 | 6.44E−04 | 1.035049 |
| 14 | rs7156328 | C | T | 0.4758 | 0.5242 | 2.58E−06 | 1.044778 |
| 14 | rs74503395 | T | G | 0.09874 | 0.90126 | 1.89E−04 | 1.055061 |
| 14 | rs112635299 | T | G | 0.02082 | 0.97918 | 3.95E−04 | 0.845126 |
| 14 | rs6575666 | T | C | 0.4003 | 0.5997 | 4.24E−04 | 1.034676 |
| 14 | rs36033161 | C | T | 0.1716 | 0.8284 | 1.49E−05 | 0.944904 |
| 14 | rs2895811 | C | T | 0.4206 | 0.5794 | 1.86E−05 | 1.041642 |
| 14 | rs4983591 | C | T | 0.1385 | 0.8615 | 8.83E−04 | 0.957601 |
| 14 | rs35246301 | G | A | 0.09422 | 0.90578 | 7.29E−04 | 0.952077 |
| 15 | rs692390 | A | G | 0.1877 | 0.8123 | 5.94E−04 | 0.951758 |
| 15 | rs1942 | A | G | 0.473 | 0.527 | 6.35E−04 | 0.966903 |
| 15 | rs59271769 | A | C | 0.07225 | 0.92775 | 2.88E−04 | 1.055189 |
| 15 | rs1996360 | G | A | 0.4812 | 0.5188 | 5.99E−04 | 1.032837 |
| 15 | rs80127393 | A | G | 0.05103 | 0.94897 | 7.66E−04 | 1.09579 |
| 15 | rs538170 | C | T | 0.3338 | 0.6662 | 5.96E−04 | 1.033496 |
| 15 | rs1800588 | T | C | 0.2156 | 0.7844 | 4.70E−04 | 1.038518 |
| 15 | rs59558657 | T | C | 0.06206 | 0.93794 | 7.85E−05 | 1.077256 |
| 15 | rs17270362 | A | G | 0.1915 | 0.8085 | 1.81E−04 | 1.047957 |
| 15 | rs6494488 | G | A | 0.1412 | 0.8588 | 8.66E−04 | 0.960397 |
| 15 | rs17293632 | T | C | 0.2371 | 0.7629 | 5.72E−09 | 0.93269 |
| 15 | rs34127110 | A | G | 0.4772 | 0.5228 | 1.89E−04 | 1.037099 |
| 15 | rs112013345 | G | T | 0.03803 | 0.96197 | 6.47E−04 | 0.893968 |
| 15 | rs1814880 | T | C | 0.2372 | 0.7628 | 1.14E−08 | 1.062754 |
| 15 | rs1994016 | T | C | 0.4242 | 0.5758 | 2.13E−13 | 0.926568 |
| 15 | rs117189732 | T | C | 0.09849 | 0.90151 | 8.96E−04 | 0.927114 |
| 15 | rs76494103 | T | G | 0.02704 | 0.97296 | 2.05E−04 | 1.103797 |
| 15 | rs7166501 | T | C | 0.3909 | 0.6091 | 2.35E−05 | 1.041073 |
| 15 | rs7174727 | G | A | 0.1525 | 0.8475 | 1.77E−06 | 1.061174 |
| 15 | rs74983338 | T | C | 0.09693 | 0.90307 | 0.00106 | 1.07537 |
| 15 | rs11636914 | T | C | 0.3327 | 0.6673 | 1.18E−05 | 1.043918 |
| 15 | rs16974612 | G | T | 0.2562 | 0.7438 | 0.001063 | 1.035646 |
| 15 | rs4843024 | A | G | 0.3274 | 0.6726 | 2.14E−04 | 0.961848 |
| 15 | rs7498010 | G | A | 0.1212 | 0.8788 | 5.55E−04 | 0.942633 |
| 15 | rs2348383 | C | T | 0.123 | 0.877 | 0.001203 | 0.958492 |
| 15 | rs79291113 | A | C | 0.01469 | 0.98531 | 5.03E−04 | 1.197343 |
| 15 | rs59383251 | G | A | 0.01084 | 0.98916 | 4.57E−05 | 0.908456 |
| 15 | rs7497304 | T | G | 0.326 | 0.674 | 2.96E−07 | 1.057118 |
| 15 | rs28593692 | A | G | 0.08118 | 0.91882 | 0.001042 | 1.071541 |
| 15 | rs8040665 | G | T | 0.2617 | 0.7383 | 9.78E−06 | 0.955676 |
| 15 | rs17506039 | A | G | 0.1524 | 0.8476 | 4.30E−04 | 0.951541 |
| 15 | rs8026716 | A | G | 0.3802 | 0.6198 | 0.001229 | 1.031903 |
| 16 | rs2076435 | T | C | 0.3155 | 0.6845 | 6.08E−04 | 1.033668 |
| 16 | rs4622512 | G | A | 0.2156 | 0.7844 | 9.80E−04 | 0.962143 |
| 16 | rs76298341 | A | C | 0.07682 | 0.92318 | 7.33E−04 | 1.060022 |
| 16 | rs80256258 | G | A | 0.08301 | 0.91699 | 6.39E−04 | 0.917853 |
| 16 | rs7184472 | T | C | 0.1852 | 0.8148 | 0.001005 | 0.957002 |
| 16 | rs1423713 | T | G | 0.04165 | 0.95835 | 6.74E−05 | 1.093425 |
| 16 | rs13332167 | C | T | 0.1558 | 0.8442 | 7.69E−04 | 1.046911 |
| 16 | rs1036696 | T | G | 0.03092 | 0.96908 | 9.98E−04 | 0.84619 |
| 16 | rs79993873 | C | A | 0.05515 | 0.94485 | 4.10E−04 | 1.094527 |
| 16 | rs2098373 | G | A | 0.375 | 0.625 | 1.54E−04 | 0.965062 |
| 16 | rs4783979 | T | C | 0.05697 | 0.94303 | 6.20E−05 | 1.088969 |
| 16 | rs2000999 | A | G | 0.1873 | 0.8127 | 7.15E−04 | 1.037782 |
| 16 | rs7186691 | T | G | 0.2939 | 0.7061 | 3.90E−04 | 0.964701 |
| 16 | rs7202877 | G | T | 0.09331 | 0.90669 | 4.22E−06 | 0.9299 |
| 16 | rs4888383 | C | T | 0.4052 | 0.5948 | 4.57E−06 | 0.95775 |
| 16 | rs72789455 | G | A | 0.09487 | 0.90513 | 0.001122 | 0.935748 |
| 16 | rs60675007 | G | A | 0.2044 | 0.7956 | 6.52E−04 | 0.958001 |
| 16 | rs66961304 | A | G | 0.1422 | 0.8578 | 3.50E−04 | 0.947502 |
| 16 | rs11649591 | A | G | 0.3781 | 0.6219 | 4.48E−05 | 1.047649 |
| 16 | rs78952982 | T | C | 0.01639 | 0.98361 | 9.59E−04 | 0.84961 |
| 16 | rs9319465 | C | T | 0.1663 | 0.8337 | 0.001053 | 1.044362 |
| 16 | rs527031 | C | T | 0.3353 | 0.6647 | 2.38E−04 | 1.036394 |
| 16 | rs75080401 | G | T | 0.01204 | 0.98796 | 8.36E−04 | 1.230103 |
| 16 | rs7196578 | T | C | 0.3309 | 0.6691 | 1.29E−04 | 0.958022 |
| 16 | rs11645860 | A | G | 0.2009 | 0.7991 | 6.11E−05 | 1.050902 |
| 17 | rs117582359 | T | C | 0.0396 | 0.9604 | 7.67E−04 | 1.090149 |
| 17 | rs1131600 | G | A | 0.1807 | 0.8193 | 0.001098 | 0.955672 |
| 17 | rs1231206 | A | G | 0.3508 | 0.6492 | 3.63E−07 | 1.049871 |
| 17 | rs216196 | C | T | 0.2768 | 0.7232 | 2.60E−06 | 0.954151 |
| 17 | rs117937130 | G | A | 0.02618 | 0.97382 | 0.001171 | 0.88236 |
| 17 | rs12940014 | T | C | 0.4738 | 0.5262 | 1.98E−04 | 1.036875 |
| 17 | rs4584886 | T | C | 0.3018 | 0.6982 | 3.16E−05 | 1.042095 |
| 17 | rs59102760 | T | C | 0.02148 | 0.97852 | 9.51E−04 | 0.866328 |
| 17 | rs9901901 | C | T | 0.4602 | 0.5398 | 8.64E−04 | 0.966699 |
| 17 | rs74866084 | T | C | 0.07464 | 0.92536 | 0.001187 | 1.081122 |
| 17 | rs13723 | G | A | 0.4921 | 0.5079 | 8.38E−05 | 1.038147 |
| 17 | rs17182658 | T | C | 0.05243 | 0.94757 | 1.90E−04 | 0.91687 |
| 17 | rs16968377 | C | T | 0.05536 | 0.94464 | 8.11E−04 | 1.062692 |
| 17 | rs9903204 | A | G | 0.2259 | 0.7741 | 1.78E−04 | 1.042426 |
| 17 | rs7210270 | G | A | 0.03168 | 0.96832 | 1.44E−05 | 1.115556 |
| 17 | rs8068844 | C | T | 0.3408 | 0.6592 | 1.64E−06 | 1.047498 |
| 17 | rs1474040 | A | G | 0.2464 | 0.7536 | 9.29E−04 | 0.959335 |
| 17 | rs7217897 | T | C | 0.4737 | 0.5263 | 5.74E−04 | 1.032259 |
| 17 | rs2668699 | G | T | 0.2158 | 0.7842 | 2.51E−05 | 1.099783 |
| 17 | rs117026337 | C | A | 0.01577 | 0.98423 | 9.41E−04 | 0.822169 |
| 17 | rs12453279 | G | T | 0.3776 | 0.6224 | 2.13E−04 | 1.035792 |
| 17 | rs1962412 | T | C | 0.3018 | 0.6982 | 7.35E−07 | 0.950917 |
| 17 | rs9674544 | G | A | 0.4805 | 0.5195 | 0.001028 | 1.031404 |
| 17 | rs12453724 | G | A | 0.2298 | 0.7702 | 7.74E−04 | 0.964915 |
| 17 | rs16948048 | G | A | 0.3689 | 0.6311 | 2.62E−05 | 1.042928 |
| 17 | rs17711010 | A | C | 0.1114 | 0.8886 | 6.45E−04 | 1.062908 |
| 17 | rs7222591 | T | G | 0.4221 | 0.5779 | 9.70E−04 | 1.032167 |
| 17 | rs8064787 | C | T | 0.1456 | 0.8544 | 1.82E−07 | 1.073838 |
| 17 | rs117297661 | A | G | 0.02762 | 0.97238 | 7.33E−04 | 1.17628 |
| 17 | rs11868441 | A | G | 0.1844 | 0.8156 | 1.73E−04 | 1.046126 |
| 17 | rs9891115 | G | A | 0.2641 | 0.7359 | 2.38E−04 | 0.960035 |
| 17 | rs6504218 | A | G | 0.4821 | 0.5179 | 3.37E−05 | 0.961834 |
| 17 | rs117567213 | C | T | 0.01359 | 0.98641 | 8.27E−04 | 1.215865 |
| 17 | rs9915486 | T | C | 0.3532 | 0.6468 | 3.33E−04 | 1.036673 |
| 17 | rs3744015 | C | T | 0.3334 | 0.6666 | 4.12E−05 | 0.959824 |
| 17 | rs8067237 | G | T | 0.2036 | 0.7964 | 0.001221 | 0.961759 |
| 17 | rs72860151 | A | G | 0.0955 | 0.9045 | 3.54E−05 | 0.896374 |
| 17 | rs8067445 | T | G | 0.4404 | 0.5596 | 5.00E−04 | 1.033037 |
| 17 | rs4062178 | C | T | 0.1915 | 0.8085 | 5.06E−04 | 0.956505 |
| 18 | rs881897 | A | C | 0.03542 | 0.96458 | 8.21E−04 | 1.064373 |
| 18 | rs66803842 | G | A | 0.1524 | 0.8476 | 7.51E−04 | 1.05113 |
| 18 | rs7228667 | T | C | 0.2427 | 0.7573 | 4.38E−05 | 1.044083 |
| 18 | rs12709787 | A | G | 0.4068 | 0.5932 | 3.50E−04 | 1.034823 |
| 18 | rs12967102 | T | C | 0.09383 | 0.90617 | 2.85E−05 | 0.946185 |
| 18 | rs58302310 | C | T | 0.01548 | 0.98452 | 2.99E−04 | 0.917488 |
| 18 | rs16977678 | T | G | 0.2872 | 0.7128 | 9.56E−04 | 1.034683 |
| 18 | rs56137770 | C | T | 0.08426 | 0.91574 | 6.98E−04 | 1.066435 |
| 18 | rs12969574 | A | G | 0.04477 | 0.95523 | 8.50E−04 | 1.104169 |
| 18 | rs328127 | C | T | 0.136 | 0.864 | 0.001006 | 1.046602 |
| 18 | rs10460078 | C | T | 0.3311 | 0.6689 | 4.60E−04 | 0.965964 |
| 18 | rs698614 | A | G | 0.327 | 0.673 | 1.07E−04 | 0.961844 |
| 18 | rs571312 | A | C | 0.2338 | 0.7662 | 3.88E−08 | 1.059416 |
| 18 | rs12969004 | A | G | 0.4124 | 0.5876 | 7.09E−04 | 1.036842 |
| 19 | rs10407866 | T | C | 0.225 | 0.775 | 0.001048 | 1.054098 |
| 19 | rs2123731 | G | A | 0.2699 | 0.7301 | 2.12E−04 | 1.039672 |
| 19 | rs72989069 | C | T | 0.04406 | 0.95594 | 6.34E−04 | 1.100401 |
| 19 | rs447135 | C | T | 0.4764 | 0.5236 | 1.54E−04 | 0.964046 |
| 19 | rs7247382 | A | C | 0.01925 | 0.98075 | 0.001238 | 1.065365 |
| 19 | rs116843064 | A | G | 0.01929 | 0.98071 | 0.001042 | 0.868678 |
| 19 | rs111425704 | T | C | 0.03046 | 0.96954 | 6.49E−04 | 0.850016 |
| 19 | rs2304165 | T | C | 0.1453 | 0.8547 | 6.40E−05 | 0.948435 |
| 19 | rs2229383 | G | T | 0.3656 | 0.6344 | 8.59E−06 | 0.957955 |
| 19 | rs4425006 | T | C | 0.09149 | 0.90851 | 1.09E−04 | 0.923388 |
| 19 | rs8101254 | T | C | 0.278 | 0.722 | 5.02E−09 | 0.938091 |
| 19 | rs55791371 | C | A | 0.1176 | 0.8824 | 4.66E−15 | 0.875266 |
| 19 | rs6511721 | G | A | 0.4812 | 0.5188 | 3.10E−08 | 1.063616 |
| 19 | rs1433099 | T | C | 0.2677 | 0.7323 | 9.77E−06 | 0.954748 |
| 19 | rs4804149 | C | T | 0.2903 | 0.7097 | 9.01E−04 | 1.042353 |
| 19 | rs10410357 | G | A | 0.1292 | 0.8708 | 5.25E−04 | 1.05066 |
| 19 | rs2108622 | T | C | 0.3016 | 0.6984 | 3.73E−06 | 1.047394 |
| 19 | rs17641567 | C | T | 0.2181 | 0.7819 | 9.28E−04 | 1.037628 |
| 19 | rs3745318 | T | C | 0.2511 | 0.7489 | 9.39E−05 | 1.048507 |
| 19 | rs12052065 | C | A | 0.3479 | 0.6521 | 3.28E−04 | 1.036337 |
| 19 | rs8101609 | T | C | 0.206 | 0.794 | 2.02E−05 | 1.052565 |
| 19 | rs111551996 | T | G | 0.05099 | 0.94901 | 1.28E−04 | 1.098885 |
| 19 | rs7252798 | T | C | 0.4321 | 0.5679 | 6.78E−04 | 0.968028 |
| 19 | rs34626245 | A | G | 0.05205 | 0.94795 | 1.83E−04 | 1.068917 |
| 19 | rs338586 | T | G | 0.442 | 0.558 | 0.001125 | 1.030734 |
| 19 | rs73045226 | G | A | 0.1219 | 0.8781 | 3.28E−07 | 1.082884 |
| 19 | rs35546772 | G | A | 0.0396 | 0.9604 | 1.83E−04 | 0.926472 |
| 19 | rs2241718 | A | G | 0.1681 | 0.8319 | 1.76E−05 | 0.946565 |
| 19 | rs4803457 | T | C | 0.3684 | 0.6316 | 1.15E−05 | 1.042194 |
| 19 | rs4525602 | G | A | 0.05223 | 0.94777 | 2.20E−04 | 1.0944 |
| 19 | rs4803717 | G | T | 0.2191 | 0.7809 | 1.37E−04 | 0.960502 |
| 19 | rs62117160 | A | G | 0.04608 | 0.95392 | 1.13E−06 | 0.857382 |
| 19 | rs3852860 | T | C | 0.4165 | 0.5835 | 1.82E−04 | 0.962126 |
| 19 | rs405509 | T | G | 0.4882 | 0.5118 | 1.74E−07 | 1.052247 |
| 19 | rs445925 | A | G | 0.109 | 0.891 | 4.23E−06 | 0.917738 |
| 19 | rs4420638 | G | A | 0.1907 | 0.8093 | 7.07E−11 | 1.096262 |
| 19 | rs12721109 | A | G | 0.0261 | 0.9739 | 6.06E−04 | 0.848729 |
| 19 | rs725660 | A | C | 0.3629 | 0.6371 | 2.92E−05 | 1.041808 |
| 19 | rs73045960 | G | A | 0.01604 | 0.98396 | 1.79E−04 | 0.861013 |
| 19 | rs11083846 | A | G | 0.2305 | 0.7695 | 5.86E−04 | 0.959548 |
| 19 | rs8105944 | T | C | 0.04516 | 0.95484 | 5.97E−04 | 1.075077 |
| 20 | rs857252 | G | T | 0.3715 | 0.6285 | 8.23E−04 | 0.967271 |
| 20 | rs6079948 | C | T | 0.2587 | 0.7413 | 8.05E−04 | 0.965336 |
| 20 | rs13734 | A | G | 0.1814 | 0.8186 | 7.65E−06 | 1.051695 |
| 20 | rs13040380 | A | G | 0.04364 | 0.95636 | 5.26E−04 | 0.907903 |
| 20 | rs6115218 | G | A | 0.2898 | 0.7102 | 6.28E−04 | 1.035426 |
| 20 | rs2424948 | T | G | 0.1147 | 0.8853 | 0.001091 | 0.948436 |
| 20 | rs6087668 | A | G | 0.01475 | 0.98525 | 5.11E−04 | 1.206885 |
| 20 | rs867186 | G | A | 0.08728 | 0.91272 | 2.92E−05 | 0.941074 |
| 20 | rs6029001 | A | C | 0.3404 | 0.6596 | 1.65E−04 | 1.03666 |
| 20 | rs2207132 | A | G | 0.03323 | 0.96677 | 1.06E−04 | 1.145914 |
| 20 | rs6129774 | A | G | 0.2068 | 0.7932 | 0.001203 | 1.036658 |
| 20 | rs6030128 | T | C | 0.08491 | 0.91509 | 2.22E−04 | 1.056724 |
| 20 | rs7270354 | A | G | 0.1456 | 0.8544 | 9.11E−05 | 1.053533 |
| 20 | rs73609696 | C | A | 0.132 | 0.868 | 6.68E−05 | 0.9443 |
| 20 | rs6512586 | G | A | 0.4373 | 0.5627 | 6.60E−05 | 0.963795 |
| 20 | rs236720 | C | A | 0.1217 | 0.8783 | 5.62E−04 | 1.048778 |
| 20 | rs117161424 | G | T | 0.07237 | 0.92763 | 1.94E−04 | 1.095861 |
| 21 | rs1474823 | G | A | 0.4764 | 0.5236 | 8.39E−04 | 1.031269 |
| 21 | rs75187018 | A | G | 0.03036 | 0.96964 | 2.89E−04 | 0.860213 |
| 21 | rs13048058 | G | A | 0.02645 | 0.97355 | 0.001118 | 1.163627 |
| 21 | rs79196774 | T | C | 0.111 | 0.889 | 7.15E−04 | 1.060563 |
| 21 | rs73203233 | A | G | 0.3829 | 0.6171 | 1.99E−04 | 0.959158 |
| 21 | rs9982601 | T | C | 0.1301 | 0.8699 | 1.33E−13 | 1.116031 |
| 21 | rs9982672 | G | A | 0.2711 | 0.7289 | 2.38E−10 | 1.073138 |
| 21 | rs117212334 | T | C | 0.03821 | 0.96179 | 6.38E−05 | 1.12791 |
| 21 | rs4819291 | G | A | 0.4034 | 0.5966 | 2.73E−04 | 0.965412 |
| 21 | rs2838762 | A | C | 0.4209 | 0.5791 | 6.45E−04 | 1.033494 |
| 21 | rs7277650 | G | A | 0.4361 | 0.5639 | 4.01E−04 | 1.034963 |
| 22 | rs9606210 | G | T | 0.2432 | 0.7568 | 0.001224 | 0.966182 |
| 22 | rs8141797 | G | A | 0.05679 | 0.94321 | 3.91E−05 | 0.932581 |
| 22 | rs57636940 | C | T | 0.01116 | 0.98884 | 2.53E−09 | 0.869082 |
| 22 | rs5760457 | C | T | 0.01452 | 0.98548 | 3.77E−05 | 0.904443 |
| 22 | rs2298389 | C | T | 0.01243 | 0.98757 | 3.70E−06 | 0.89501 |
| 22 | rs738996 | T | C | 0.2051 | 0.7949 | 0.001037 | 1.044674 |
| 22 | rs2530664 | C | A | 0.179 | 0.821 | 5.61E−04 | 1.042815 |
| 22 | rs6006426 | G | A | 0.4605 | 0.5395 | 1.00E−06 | 0.954707 |
| 22 | rs5749088 | T | C | 0.2407 | 0.7593 | 0.001134 | 1.03843 |
| 22 | rs5994619 | T | C | 0.1849 | 0.8151 | 0.001186 | 0.964055 |
| 22 | rs13056230 | T | C | 0.4276 | 0.5724 | 0.001134 | 1.032757 |
| 22 | rs73174419 | A | G | 0.06046 | 0.93954 | 3.11E−04 | 0.909261 |

The present invention also provides a method for assessing the risk of a human subject developing coronary artery disease, the method comprising performing a genetic risk assessment of the human subject, wherein the genetic risk assessment involves detecting, in a biological sample derived from the human subject, the presence of at least one polymorphisms associated with a risk of developing coronary artery disease, wherein the at least one polymorphism is selected from Table 2, or a single nucleotide polymorphism in linkage disequilibrium with one or more thereof. In an embodiment, the method comprises detecting the presence of at least two, at least three, at least four, at least five, at least six, at least seven, at least eight, at least nine, at least ten, at least 20, at least 50, at least 100, at least 125, or all of the polymorphism provided in Table 2, or a single nucleotide polymorphism in linkage disequilibrium with one or more thereof. In embodiment, the method comprises detecting each of the polymorphisms provided in Table 2.

The present invention also provides a method for assessing the risk of a human subject developing coronary artery disease, the method comprising performing a genetic risk assessment of the human subject, wherein the genetic risk assessment involves detecting, in a biological sample derived from the human subject, the presence of at least one polymorphisms associated with a risk of developing coronary artery disease, wherein the at least one polymorphism is selected from Table 1 and/or Table 2, or a single nucleotide polymorphism in linkage disequilibrium with one or more thereof. In an embodiment, the method comprises detecting each of the polymorphisms provided in Tables 1 and 2.

TABLE 2
Further polymorphisms associated with coronary
artery disease. A1 is the risk allele.

| CHR | SNP | A1 | A2 | A1_freq | A2_freq | p_value | OR |
|---|---|---|---|---|---|---|---|
| 1 | rs2132303 | T | C | 0.1445 | 0.8555 | 9.18E−04 | 1.044628 |
| 1 | rs28739509 | C | T | 0.2751 | 0.7249 | 6.57E−04 | 1.038945 |
| 1 | rs28545785 | C | T | 0.1342 | 0.8658 | 4.54E−05 | 0.939639 |
| 1 | rs7513411 | T | G | 0.205 | 0.795 | 3.91E−05 | 0.951648 |
| 1 | rs10782635 | T | C | 0.459 | 0.541 | 5.54E−04 | 0.968427 |
| 1 | rs6698843 | T | C | 0.4602 | 0.5398 | 4.25E−05 | 1.040486 |
| 1 | rs11577931 | G | A | 0.0911 | 0.9089 | 2.84E−07 | 0.905951 |
| 1 | rs55660224 | T | C | 0.08334 | 0.91666 | 4.79E−05 | 0.925928 |
| 1 | rs76186504 | T | C | 0.02533 | 0.97467 | 9.80E−04 | 0.887715 |
| 1 | rs17646665 | G | A | 0.06474 | 0.93526 | 1.25E−04 | 0.9205 |
| 1 | rs61812598 | A | G | 0.4097 | 0.5903 | 1.69E−07 | 0.95117 |
| 1 | rs4240872 | C | T | 0.2241 | 0.7759 | 6.73E−04 | 1.037301 |
| 1 | rs11264296 | C | T | 0.4167 | 0.5833 | 4.02E−04 | 1.034965 |
| 1 | rs11265297 | C | T | 0.4343 | 0.5657 | 8.31E−04 | 1.0321 |
| 1 | rs4846382 | C | T | 0.4019 | 0.5981 | 1.35E−04 | 0.957959 |
| 1 | rs74148131 | T | G | 0.281 | 0.719 | 2.34E−04 | 0.956694 |
| 1 | rs3002141 | T | C | 0.1869 | 0.8131 | 1.24E−06 | 0.939804 |
| 1 | rs17531063 | G | T | 0.09542 | 0.90458 | 1.28E−05 | 0.911722 |
| 1 | rs4264001 | G | T | 0.254 | 0.746 | 2.43E−04 | 0.953672 |
| 1 | rs3811445 | A | G | 0.4191 | 0.5809 | 0.001102 | 0.968939 |
| 2 | rs17522184 | T | C | 0.2143 | 0.7857 | 4.30E−04 | 1.042953 |
| 2 | rs386397 | A | G | 0.3126 | 0.6874 | 7.68E−04 | 1.035986 |
| 2 | rs10166144 | G | A | 0.1242 | 0.8758 | 1.42E−06 | 0.938694 |
| 2 | rs10865262 | T | C | 0.4699 | 0.5301 | 2.21E−04 | 0.966446 |
| 2 | rs17046192 | A | G | 0.483 | 0.517 | 5.08E−04 | 1.033692 |
| 2 | rs3731828 | T | C | 0.2812 | 0.7188 | 1.48E−07 | 1.057404 |
| 2 | rs6733550 | G | T | 0.3626 | 0.6374 | 2.69E−04 | 1.036829 |
| 2 | rs6430059 | A | G | 0.3111 | 0.6889 | 1.41E−04 | 1.038669 |
| 2 | rs79391583 | T | G | 0.02212 | 0.97788 | 1.32E−06 | 1.198603 |
| 2 | rs786280 | C | T | 0.2879 | 0.7121 | 0.001155 | 1.036049 |
| 2 | rs1425047 | G | A | 0.4224 | 0.5776 | 7.76E−04 | 1.032347 |
| 2 | Affx- | A | C | 0.3541 | 0.6459 | 6.01E−04 | 1.034637 |
| | 18522969 | | | | | | |
| 2 | rs78488377 | A | G | 0.04449 | 0.95551 | 1.78E−09 | 1.177982 |
| 2 | rs72926788 | C | T | 0.0379 | 0.9621 | 4.63E−07 | 1.169756 |
| 2 | rs2571445 | A | G | 0.3928 | 0.6072 | 1.23E−05 | 1.043586 |
| 2 | rs16858298 | G | A | 0.2463 | 0.7537 | 6.22E−04 | 0.962895 |
| 3 | rs35761247 | A | G | 0.05884 | 0.94116 | 9.42E−04 | 0.923214 |
| 3 | rs116503914 | G | T | 0.04544 | 0.95456 | 1.74E−04 | 0.889986 |
| 3 | rs4625 | G | A | 0.3028 | 0.6972 | 2.23E−05 | 0.957472 |
| 3 | rs2581778 | A | G | 0.4261 | 0.5739 | 6.35E−04 | 1.032116 |
| 3 | rs6438025 | A | G | 0.3592 | 0.6408 | 0.001192 | 0.970083 |
| 3 | rs870343 | C | T | 0.3397 | 0.6603 | 9.80E−04 | 1.032905 |
| 3 | rs645040 | G | T | 0.2253 | 0.7747 | 6.03E−04 | 0.96215 |
| 3 | rs9845672 | G | A | 0.3464 | 0.6536 | 3.60E−05 | 1.040711 |
| 3 | rs1679153 | G | A | 0.08825 | 0.91175 | 0.001093 | 1.047896 |
| 4 | rs28560455 | T | C | 0.1362 | 0.8638 | 0.001234 | 0.951382 |
| 4 | rs2452600 | T | C | 0.3152 | 0.6848 | 2.55E−05 | 0.957354 |
| 4 | rs12639887 | T | C | 0.4181 | 0.5819 | 0.001033 | 1.0312 |
| 4 | rs7436506 | C | T | 0.3002 | 0.6998 | 3.60E−04 | 0.96328 |
| 4 | rs55851967 | G | A | 0.3102 | 0.6898 | 1.29E−05 | 1.045481 |
| 4 | rs4345206 | T | C | 0.4794 | 0.5206 | 6.00E−04 | 1.033186 |
| 4 | rs12644017 | A | G | 0.1495 | 0.8505 | 3.87E−06 | 0.944607 |
| 4 | rs113436562 | T | C | 0.05478 | 0.94522 | 2.65E−06 | 0.884007 |
| 4 | rs80010398 | T | C | 0.04711 | 0.95289 | 3.99E−04 | 1.067668 |
| 4 | rs28437901 | A | G | 0.05752 | 0.94248 | 0.001117 | 1.05074 |
| 4 | rs17033388 | C | T | 0.4356 | 0.5644 | 4.32E−04 | 1.033145 |
| 4 | rs12502903 | A | G | 0.1975 | 0.8025 | 7.82E−07 | 0.943812 |
| 5 | rs937218 | G | A | 0.4482 | 0.5518 | 7.86E−05 | 1.037194 |
| 5 | rs1493470 | A | G | 0.3085 | 0.6915 | 8.22E−04 | 0.963854 |
| 5 | rs288194 | T | C | 0.4382 | 0.5618 | 2.38E−04 | 0.96599 |
| 5 | rs288173 | G | A | 0.1734 | 0.8266 | 6.74E−05 | 0.955566 |
| 5 | rs890928 | A | C | 0.2924 | 0.7076 | 2.58E−04 | 1.038565 |
| 5 | rs1384880 | T | C | 0.4014 | 0.5986 | 4.31E−04 | 1.034176 |
| 5 | rs4912610 | C | T | 0.1512 | 0.8488 | 8.66E−04 | 1.04207 |
| 6 | rs79231131 | G | A | 0.02524 | 0.97476 | 0.001203 | 0.865516 |
| 6 | rs12202891 | T | C | 0.1814 | 0.8186 | 9.65E−09 | 1.079542 |
| 6 | rs12215208 | T | C | 0.4573 | 0.5427 | 5.68E−14 | 1.073218 |
| 6 | rs9381462 | A | G | 0.4954 | 0.5046 | 2.47E−11 | 0.937171 |
| 6 | rs9369640 | C | A | 0.3626 | 0.6374 | 7.78E−19 | 0.914749 |
| 6 | rs35056688 | T | C | 0.2326 | 0.7674 | 4.89E−06 | 1.051628 |
| 6 | rs80276151 | A | G | 0.03103 | 0.96897 | 6.68E−04 | 0.854563 |
| 6 | rs4389765 | C | T | 0.2243 | 0.7757 | 1.37E−04 | 0.955139 |
| 6 | rs72833643 | G | A | 0.04069 | 0.95931 | 6.00E−04 | 1.11925 |
| 6 | rs62399429 | A | G | 0.1466 | 0.8534 | 5.34E−04 | 1.051944 |
| 6 | rs10947129 | A | G | 0.1453 | 0.8547 | 1.44E−05 | 1.060524 |
| 6 | rs2517523 | G | A | 0.3551 | 0.6449 | 0.001072 | 1.035688 |
| 6 | rs9263969 | T | C | 0.2181 | 0.7819 | 4.85E−04 | 1.042341 |
| 6 | rs2523608 | G | A | 0.4021 | 0.5979 | 0.00123 | 0.963487 |
| 6 | rs9266772 | C | T | 0.2136 | 0.7864 | 2.92E−04 | 1.049017 |
| 6 | rs3093998 | C | A | 0.3443 | 0.6557 | 8.59E−04 | 0.965229 |
| 6 | rs2815095 | T | C | 0.2631 | 0.7369 | 1.83E−04 | 1.04072 |
| 6 | rs1885659 | A | G | 0.2632 | 0.7368 | 2.79E−04 | 0.955706 |
| 6 | rs72910872 | A | G | 0.05309 | 0.94691 | 0.001099 | 0.915961 |
| 6 | rs915125 | T | C | 0.2767 | 0.7233 | 2.02E−04 | 1.037514 |
| 6 | rs11965046 | C | T | 0.2777 | 0.7223 | 9.91E−04 | 0.964825 |
| 6 | rs2273621 | G | A | 0.3202 | 0.6798 | 4.19E−04 | 0.966799 |
| 6 | rs9490318 | T | C | 0.1452 | 0.8548 | 8.19E−04 | 0.957142 |
| 6 | rs2282143 | T | C | 0.01558 | 0.98442 | 1.31E−06 | 1.262738 |
| 6 | rs4709408 | T | C | 0.04012 | 0.95988 | 9.69E−08 | 1.09286 |
| 6 | rs3861973 | T | C | 0.2666 | 0.7334 | 0.001061 | 0.959412 |
| 6 | rs932925 | A | G | 0.1581 | 0.8419 | 5.82E−09 | 1.085613 |
| 6 | rs372665 | G | A | 0.3072 | 0.6928 | 1.16E−04 | 0.962791 |
| 6 | rs3127599 | T | C | 0.3052 | 0.6948 | 7.85E−04 | 1.035002 |
| 6 | rs7770628 | C | T | 0.4684 | 0.5316 | 7.52E−09 | 1.057168 |
| 6 | rs1740428 | A | G | 0.3637 | 0.6363 | 1.04E−05 | 0.959298 |
| 7 | rs73683436 | C | T | 0.2814 | 0.7186 | 2.82E−04 | 1.036192 |
| 7 | rs2283017 | G | A | 0.391 | 0.609 | 8.32E−04 | 0.968684 |
| 7 | rs12216661 | G | A | 0.4123 | 0.5877 | 3.42E−04 | 1.035307 |
| 8 | rs7816032 | T | C | 0.2356 | 0.7644 | 1.06E−04 | 0.956541 |
| 8 | rs11991231 | C | T | 0.104 | 0.896 | 6.88E−04 | 0.947116 |
| 8 | rs2954033 | A | G | 0.302 | 0.698 | 8.70E−06 | 1.046512 |
| 9 | rs3731239 | G | A | 0.3675 | 0.6325 | 7.55E−14 | 0.924853 |
| 9 | rs3217992 | T | C | 0.3645 | 0.6355 | 1.03E−42 | 1.136913 |
| 9 | rs1412829 | G | A | 0.425 | 0.575 | 2.19E−35 | 0.885027 |
| 9 | rs1011970 | T | G | 0.1703 | 0.8297 | 2.88E−04 | 0.957412 |
| 9 | rs116237463 | A | G | 0.06697 | 0.93303 | 0.001144 | 1.084586 |
| 9 | rs2066715 | T | C | 0.0632 | 0.9368 | 2.05E−04 | 1.066794 |
| 9 | rs10979023 | A | G | 0.1864 | 0.8136 | 0.001197 | 1.04251 |
| 9 | rs10979032 | G | T | 0.4394 | 0.5606 | 5.08E−05 | 0.961318 |
| 9 | rs35931893 | T | C | 0.04806 | 0.95194 | 5.10E−04 | 1.115297 |
| 9 | rs74971632 | T | C | 0.2681 | 0.7319 | 1.08E−04 | 1.044501 |
| 9 | rs11244035 | T | C | 0.1056 | 0.8944 | 3.62E−05 | 1.083237 |
| 9 | rs612169 | G | A | 0.318 | 0.682 | 4.55E−06 | 1.044326 |
| 9 | rs11244084 | T | C | 0.07292 | 0.92708 | 4.32E−06 | 1.107295 |
| 9 | rs56343119 | A | C | 0.1391 | 0.8609 | 3.09E−06 | 1.077382 |
| 10 | rs11007851 | T | C | 0.4668 | 0.5332 | 2.55E−08 | 0.948023 |
| 10 | rs2185724 | C | T | 0.4234 | 0.5766 | 7.75E−07 | 0.953737 |
| 10 | rs2887510 | A | G | 0.3201 | 0.6799 | 5.11E−04 | 0.966818 |
| 10 | rs7923335 | C | A | 0.2147 | 0.7853 | 3.90E−11 | 0.928566 |
| 10 | rs268274 | C | T | 0.1996 | 0.8004 | 2.62E−05 | 0.953254 |
| 10 | rs1746043 | C | T | 0.3517 | 0.6483 | 4.11E−07 | 0.953047 |
| 10 | rs11238956 | C | T | 0.3523 | 0.6477 | 5.09E−07 | 1.056221 |
| 10 | rs1144478 | T | C | 0.4853 | 0.5147 | 3.99E−04 | 0.967746 |
| 10 | rs17680430 | G | A | 0.1107 | 0.8893 | 6.09E−04 | 1.06172 |
| 10 | rs12257915 | C | T | 0.4247 | 0.5753 | 9.40E−05 | 1.036905 |
| 10 | rs11591797 | G | A | 0.2647 | 0.7353 | 0.001186 | 0.967258 |
| 10 | rs8354 | T | C | 0.07943 | 0.92057 | 3.01E−05 | 0.944899 |
| 10 | rs12764049 | G | A | 0.2233 | 0.7767 | 3.09E−04 | 0.962544 |
| 10 | rs7914558 | A | G | 0.3889 | 0.6111 | 1.74E−04 | 0.965775 |
| 11 | rs11042219 | T | C | 0.07847 | 0.92153 | 7.29E−04 | 1.060384 |
| 11 | rs61878123 | A | G | 0.2689 | 0.7311 | 8.47E−06 | 0.94674 |
| 11 | rs7947951 | A | G | 0.3075 | 0.6925 | 4.51E−04 | 0.966236 |
| 11 | rs17095428 | C | A | 0.3072 | 0.6928 | 7.90E−05 | 1.043176 |
| 11 | rs10791605 | G | T | 0.374 | 0.626 | 7.40E−04 | 1.03359 |
| 11 | rs750338 | G | A | 0.2261 | 0.7739 | 7.74E−04 | 1.037947 |
| 12 | rs11044977 | T | C | 0.3848 | 0.6152 | 0.001067 | 1.032985 |
| 12 | Affx- | A | C | 0.2365 | 0.7635 | 7.70E−04 | 0.965702 |
| | 35720446 | | | | | | |
| 12 | rs11181289 | T | C | 0.3836 | 0.6164 | 4.52E−04 | 1.033742 |
| 12 | rs17666080 | C | T | 0.1092 | 0.8908 | 0.001096 | 1.060262 |
| 12 | rs2694829 | T | C | 0.1416 | 0.8584 | 0.001104 | 0.957477 |
| 12 | rs10858894 | C | T | 0.2365 | 0.7635 | 4.29E−06 | 1.050658 |
| 12 | rs4842665 | A | G | 0.3964 | 0.6036 | 0.001041 | 1.034744 |
| 12 | rs7136259 | T | C | 0.4179 | 0.5821 | 2.45E−05 | 1.040142 |
| 12 | rs11105381 | A | G | 0.186 | 0.814 | 0.001057 | 1.038084 |
| 12 | rs1265565 | T | C | 0.375 | 0.625 | 3.10E−05 | 1.049326 |
| 12 | rs1265566 | C | T | 0.3141 | 0.6859 | 0.001063 | 0.968743 |
| 12 | rs7970490 | G | A | 0.2862 | 0.7138 | 3.02E−04 | 0.963086 |
| 12 | rs2238151 | C | T | 0.325 | 0.675 | 1.06E−05 | 0.955838 |
| 12 | rs79680080 | C | A | 0.02684 | 0.97316 | 0.00121 | 0.894596 |
| 12 | rs2133638 | T | C | 0.3322 | 0.6678 | 7.28E−04 | 1.034255 |
| 13 | rs9513112 | A | G | 0.3268 | 0.6732 | 1.12E−05 | 1.043947 |
| 13 | rs2166624 | A | G | 0.3601 | 0.6399 | 6.83E−04 | 0.966638 |
| 13 | rs59504364 | A | G | 0.4546 | 0.5454 | 0.001154 | 0.969707 |
| 13 | rs2131939 | A | G | 0.1384 | 0.8616 | 5.41E−04 | 1.050359 |
| 13 | rs12858728 | T | C | 0.1268 | 0.8732 | 6.69E−04 | 1.052378 |
| 13 | rs6492259 | T | G | 0.06232 | 0.93768 | 6.49E−05 | 1.097034 |
| 13 | rs7989565 | G | A | 0.1388 | 0.8612 | 3.71E−04 | 0.946997 |
| 13 | rs9521732 | A | C | 0.3802 | 0.6198 | 6.51E−04 | 1.032638 |
| 14 | rs56096740 | A | C | 0.1179 | 0.8821 | 6.17E−04 | 1.050212 |
| 14 | rs12435918 | G | A | 0.3137 | 0.6863 | 7.46E−04 | 1.033961 |
| 15 | rs1704345 | C | T | 0.1117 | 0.8883 | 8.38E−04 | 1.046606 |
| 15 | rs17293443 | C | T | 0.2289 | 0.7711 | 3.44E−06 | 0.945944 |
| 15 | rs744910 | G | A | 0.4847 | 0.5153 | 6.79E−04 | 0.969166 |
| 15 | rs1996371 | C | T | 0.4086 | 0.5914 | 8.32E−05 | 0.961199 |
| 15 | rs11072783 | G | A | 0.163 | 0.837 | 5.81E−04 | 1.03861 |
| 15 | rs2277547 | G | A | 0.2554 | 0.7446 | 1.71E−11 | 1.069939 |
| 15 | rs74608880 | A | G | 0.05541 | 0.94459 | 1.56E−04 | 1.102766 |
| 15 | rs8035039 | A | G | 0.4948 | 0.5052 | 1.69E−09 | 0.944326 |
| 15 | rs8034658 | T | G | 0.2758 | 0.7242 | 2.07E−04 | 1.042598 |
| 15 | rs9302286 | G | A | 0.09588 | 0.90412 | 7.78E−04 | 1.051738 |
| 15 | rs12900736 | T | C | 0.1705 | 0.8295 | 6.13E−04 | 0.957361 |
| 15 | rs78029429 | T | C | 0.02295 | 0.97705 | 7.70E−04 | 1.171641 |
| 15 | rs4702 | G | A | 0.4436 | 0.5564 | 2.45E−04 | 0.96313 |
| 15 | rs2677738 | A | G | 0.3514 | 0.6486 | 7.50E−06 | 0.955329 |
| 16 | rs72802340 | T | G | 0.03811 | 0.96189 | 2.07E−04 | 0.858726 |
| 16 | rs7190458 | A | G | 0.04474 | 0.95526 | 9.74E−04 | 0.913173 |
| 16 | rs56258397 | T | G | 0.09545 | 0.90455 | 6.48E−05 | 0.91898 |
| 17 | rs2760734 | T | C | 0.1681 | 0.8319 | 3.19E−04 | 1.043815 |
| 17 | rs12936587 | A | G | 0.4741 | 0.5259 | 8.24E−04 | 0.967274 |
| 17 | rs2006933 | G | T | 0.2351 | 0.7649 | 9.61E−04 | 0.965168 |
| 17 | rs11657079 | T | C | 0.2547 | 0.7453 | 9.29E−04 | 1.040978 |
| 17 | rs62075809 | T | C | 0.3729 | 0.6271 | 3.00E−05 | 0.96042 |
| 17 | rs28659819 | A | G | 0.3076 | 0.6924 | 7.80E−04 | 1.033881 |
| 17 | rs17608766 | C | T | 0.1479 | 0.8521 | 4.94E−04 | 1.054417 |
| 17 | rs15563 | A | G | 0.47 | 0.53 | 1.83E−05 | 0.961035 |
| 17 | rs4794008 | T | C | 0.2865 | 0.7135 | 0.001002 | 0.961308 |
| 17 | rs11652684 | G | T | 0.1986 | 0.8014 | 6.79E−04 | 0.954113 |
| 17 | rs9916472 | T | C | 0.3262 | 0.6738 | 2.46E−06 | 0.952311 |
| 17 | rs4987082 | C | T | 0.4332 | 0.5668 | 3.93E−04 | 1.035465 |
| 17 | rs1867625 | A | G | 0.2802 | 0.7198 | 3.55E−04 | 0.964044 |
| 17 | rs8081539 | G | T | 0.3507 | 0.6493 | 2.59E−04 | 0.964148 |
| 17 | rs4398150 | T | C | 0.1029 | 0.8971 | 4.31E−04 | 0.94781 |
| 18 | rs9951447 | C | T | 0.3961 | 0.6039 | 2.17E−04 | 1.035616 |
| 18 | rs2110135 | A | C | 0.491 | 0.509 | 5.56E−04 | 1.032782 |
| 18 | rs4121765 | G | A | 0.3034 | 0.6966 | 7.59E−05 | 1.04069 |
| 18 | rs589850 | A | G | 0.4382 | 0.5618 | 2.88E−04 | 1.034606 |
| 18 | rs35922794 | C | T | 0.09048 | 0.90952 | 5.99E−04 | 1.063175 |
| 19 | rs8106664 | G | T | 0.2194 | 0.7806 | 4.64E−04 | 0.961214 |
| 19 | rs10775614 | G | A | 0.3515 | 0.6485 | 5.02E−05 | 0.959474 |
| 19 | rs4804528 | T | G | 0.448 | 0.552 | 3.03E−05 | 0.960748 |
| 19 | rs2304087 | C | T | 0.372 | 0.628 | 1.02E−06 | 0.952969 |
| 19 | rs73013159 | T | G | 0.06582 | 0.93418 | 2.35E−08 | 0.877073 |
| 19 | rs3786721 | T | C | 0.4422 | 0.5578 | 9.68E−04 | 1.032309 |
| 19 | rs1122608 | T | G | 0.2548 | 0.7452 | 2.73E−11 | 0.929376 |
| 19 | rs11668477 | G | A | 0.2083 | 0.7917 | 2.28E−06 | 0.942764 |
| 19 | rs1799898 | T | C | 0.1289 | 0.8711 | 2.52E−04 | 0.949388 |
| 19 | rs34645805 | G | A | 0.4458 | 0.5542 | 1.27E−04 | 1.038317 |
| 19 | rs3093105 | C | A | 0.1634 | 0.8366 | 9.75E−05 | 1.049456 |
| 19 | rs533086 | A | G | 0.2673 | 0.7327 | 2.91E−04 | 1.045202 |
| 19 | rs7253117 | G | A | 0.4965 | 0.5035 | 4.17E−05 | 1.04127 |
| 19 | rs11670056 | T | C | 0.07525 | 0.92475 | 6.77E−04 | 1.072317 |
| 19 | rs10853751 | G | A | 0.3963 | 0.6037 | 6.51E−04 | 1.032113 |
| 19 | rs2075650 | G | A | 0.1467 | 0.8533 | 1.61E−06 | 1.073701 |
| 19 | rs874462 | A | G | 0.3536 | 0.6464 | 7.31E−04 | 0.965941 |
| 20 | rs4141586 | T | C | 0.3466 | 0.6534 | 0.001169 | 1.033353 |
| 21 | rs7276739 | A | G | 0.1019 | 0.8981 | 1.59E−06 | 1.087471 |
| 21 | rs2834419 | C | T | 0.2915 | 0.7085 | 0.001053 | 1.033032 |
| 21 | rs79158929 | A | G | 0.07691 | 0.92309 | 3.64E−10 | 1.136625 |
| 22 | rs7293001 | A | G | 0.02523 | 0.97477 | 4.45E−06 | 0.913412 |
| 22 | rs6006427 | C | T | 0.2377 | 0.7623 | 1.22E−04 | 0.959875 |

The present invention also provides a method for assessing the risk of a human subject developing atrial fibrillation, the method comprising performing a genetic risk assessment of the human subject, wherein the genetic risk assessment involves detecting, in a biological sample derived from the human subject, the presence at least one polymorphisms associated with a risk of developing atrial fibrillation, wherein the at least one polymorphism is selected from Table 3, or a single nucleotide polymorphism in linkage disequilibrium with one or more thereof. In an embodiment, the method comprises detecting the presence of at least two, at least three, at least four, at least five, at least six, at least seven, at least eight, at least nine, at least ten, at least 20, at least 50, at least 100, at least 125, at least 150, at least 175, at least 200, at least 225, at least 250, or all of the polymorphism provided in Table 3, or a single nucleotide polymorphism in linkage disequilibrium with one or more thereof. In an embodiment, the method comprises detecting each of the polymorphism provided in Table 3.

TABLE 3
Polymorphisms associated with atrial fibrillation. A1 is the risk allele.

| CHR | SNP | A1 | A2 | A1_freq | A2_freq | p_value | OR |
|---|---|---|---|---|---|---|---|
| 1 | rs6593549 | C | T | 0.09644 | 0.90356 | 5.14E−05 | 1.082746 |
| 1 | rs4074536 | C | T | 0.2939 | 0.7061 | 4.47E−05 | 0.945728 |
| 1 | rs76968567 | T | C | 0.0528 | 0.9472 | 2.18E−05 | 0.87564 |
| 1 | rs3806234 | A | G | 0.4072 | 0.5928 | 3.95E−05 | 1.054219 |
| 1 | rs7345 | G | T | 0.4676 | 0.5324 | 3.81E−05 | 0.950089 |
| 1 | rs10797096 | A | G | 0.4616 | 0.5384 | 1.23E−05 | 1.055801 |
| 1 | rs7549250 | C | T | 0.4197 | 0.5803 | 1.76E−05 | 1.054746 |
| 1 | rs4845625 | T | C | 0.4212 | 0.5788 | 1.54E−05 | 1.054852 |
| 1 | rs4129267 | T | C | 0.4092 | 0.5908 | 3.15E−05 | 0.94838 |
| 1 | rs2229238 | T | C | 0.183 | 0.817 | 4.58E−06 | 1.071972 |
| 1 | rs13376333 | T | C | 0.3158 | 0.6842 | 2.65E−12 | 1.100209 |
| 1 | rs6658392 | C | T | 0.3512 | 0.6488 | 6.63E−12 | 1.09505 |
| 1 | rs76102976 | A | G | 0.124 | 0.876 | 5.45E−05 | 0.927002 |
| 1 | rs4845396 | A | G | 0.4828 | 0.5172 | 4.54E−05 | 0.950184 |
| 1 | rs10908444 | A | G | 0.4195 | 0.5805 | 3.37E−07 | 0.936224 |
| 1 | rs2335407 | T | C | 0.3903 | 0.6097 | 6.23E−06 | 0.94299 |
| 1 | rs4845695 | G | A | 0.3643 | 0.6357 | 1.83E−05 | 0.944689 |
| 1 | rs41264253 | A | G | 0.1094 | 0.8906 | 1.47E−10 | 1.162648 |
| 1 | rs2061690 | T | C | 0.4236 | 0.5764 | 4.86E−06 | 0.943178 |
| 1 | rs61811895 | T | G | 0.1757 | 0.8243 | 4.08E−09 | 1.119856 |
| 1 | rs11264296 | C | T | 0.4167 | 0.5833 | 4.55E−07 | 0.937911 |
| 1 | rs11264303 | A | C | 0.4653 | 0.5347 | 2.00E−05 | 0.945066 |
| 1 | rs41264285 | T | C | 0.2221 | 0.7779 | 3.31E−05 | 1.069295 |
| 1 | rs56675301 | C | T | 0.2686 | 0.7314 | 3.64E−05 | 1.060563 |
| 1 | rs2297775 | C | T | 0.2992 | 0.7008 | 5.65E−05 | 1.055801 |
| 1 | rs79858200 | T | C | 0.03853 | 0.96147 | 3.87E−05 | 1.146943 |
| 1 | rs41272485 | G | A | 0.03862 | 0.96138 | 5.56E−05 | 1.14385 |
| 1 | rs10919326 | G | A | 0.3351 | 0.6649 | 3.73E−05 | 0.94535 |
| 1 | rs4399218 | T | G | 0.3204 | 0.6796 | 2.59E−07 | 0.931835 |
| 1 | rs72700121 | G | A | 0.1223 | 0.8777 | 3.77E−11 | 1.142136 |
| 1 | rs11581269 | G | T | 0.1426 | 0.8574 | 2.63E−05 | 1.083287 |
| 1 | rs6427245 | C | T | 0.3916 | 0.6084 | 2.18E−05 | 0.947811 |
| 1 | rs7522128 | T | C | 0.2229 | 0.7771 | 1.35E−05 | 1.065666 |
| 1 | rs736791 | A | G | 0.4426 | 0.5574 | 5.69E−13 | 1.094174 |
| 1 | rs12566725 | T | C | 0.2975 | 0.7025 | 1.93E−13 | 0.905471 |
| 1 | rs56250774 | G | A | 0.1847 | 0.8153 | 1.39E−06 | 1.084696 |
| 1 | rs659580 | T | C | 0.2621 | 0.7379 | 6.02E−07 | 1.074226 |
| 1 | rs525489 | T | G | 0.4461 | 0.5539 | 1.19E−13 | 1.097571 |
| 1 | rs502612 | C | T | 0.4814 | 0.5186 | 5.68E−07 | 0.938662 |
| 1 | rs4656794 | A | G | 0.3711 | 0.6289 | 9.53E−08 | 0.932301 |
| 1 | rs719414 | G | A | 0.2069 | 0.7931 | 1.37E−05 | 1.0781 |
| 2 | rs2540970 | C | T | 0.3255 | 0.6745 | 3.77E−07 | 0.935944 |
| 2 | rs2723065 | G | A | 0.3736 | 0.6264 | 7.63E−10 | 0.924872 |
| 2 | rs2249105 | G | A | 0.3611 | 0.6389 | 7.22E−09 | 0.928765 |
| 2 | rs2241160 | G | A | 0.4142 | 0.5858 | 1.23E−08 | 0.931462 |
| 2 | rs2723091 | T | C | 0.3007 | 0.6993 | 1.12E−05 | 0.943084 |
| 2 | rs13421845 | G | A | 0.3318 | 0.6682 | 9.35E−07 | 0.937724 |
| 2 | rs56718363 | C | T | 0.1469 | 0.8531 | 2.99E−05 | 0.929043 |
| 2 | rs6546544 | C | T | 0.1245 | 0.8755 | 6.41E−06 | 0.923948 |
| 2 | rs7567400 | A | G | 0.4905 | 0.5095 | 4.58E−09 | 0.929601 |
| 2 | rs2228203 | T | C | 0.1954 | 0.8046 | 9.98E−10 | 0.909373 |
| 2 | rs2168115 | G | A | 0.4566 | 0.5434 | 8.35E−11 | 0.921917 |
| 2 | rs6546559 | A | G | 0.4342 | 0.5658 | 6.78E−09 | 0.930624 |
| 2 | rs11686934 | G | A | 0.4464 | 0.5536 | 1.44E−08 | 0.929973 |
| 2 | rs7581977 | G | A | 0.4343 | 0.5657 | 2.38E−08 | 0.930531 |
| 2 | rs3796097 | T | C | 0.4735 | 0.5265 | 1.61E−05 | 1.059927 |
| 2 | rs7585413 | G | A | 0.3098 | 0.6902 | 3.80E−05 | 0.946485 |
| 2 | rs767984 | C | T | 0.3032 | 0.6968 | 4.89E−05 | 0.94658 |
| 2 | rs11890277 | T | C | 0.1048 | 0.8952 | 2.91E−05 | 1.084371 |
| 2 | rs1375132 | G | A | 0.1957 | 0.8043 | 1.92E−05 | 0.939883 |
| 2 | rs34985138 | T | C | 0.07336 | 0.92664 | 1.14E−05 | 1.105171 |
| 2 | rs9967820 | G | T | 0.1431 | 0.8569 | 9.19E−06 | 1.07713 |
| 2 | rs3829748 | A | G | 0.136 | 0.864 | 3.08E−07 | 1.08937 |
| 2 | rs35504893 | T | C | 0.1771 | 0.8229 | 4.29E−08 | 1.086433 |
| 2 | rs2366751 | G | A | 0.2138 | 0.7862 | 3.63E−06 | 1.068654 |
| 2 | rs3731746 | A | G | 0.1559 | 0.8441 | 2.70E−08 | 1.09177 |
| 2 | rs2303838 | T | C | 0.1648 | 0.8352 | 3.14E−07 | 1.082746 |
| 2 | rs2288569 | T | C | 0.1361 | 0.8639 | 3.64E−06 | 1.081663 |
| 2 | rs2042995 | C | T | 0.2212 | 0.7788 | 4.55E−05 | 1.060245 |
| 2 | rs16866465 | G | T | 0.1534 | 0.8466 | 3.16E−06 | 1.081339 |
| 2 | rs17588027 | A | G | 0.1398 | 0.8602 | 4.71E−05 | 1.081231 |
| 2 | rs295137 | T | C | 0.4012 | 0.5988 | 1.90E−06 | 0.939977 |
| 2 | rs7578220 | G | A | 0.4285 | 0.5715 | 4.64E−06 | 1.059079 |
| 3 | rs3901666 | A | C | 0.3965 | 0.6035 | 3.28E−05 | 0.948096 |
| 3 | rs11718898 | T | C | 0.333 | 0.667 | 4.68E−08 | 0.929322 |
| 3 | rs2305398 | A | G | 0.381 | 0.619 | 3.86E−05 | 0.947243 |
| 3 | rs3732675 | T | C | 0.4134 | 0.5866 | 3.42E−05 | 0.948285 |
| 3 | rs3732678 | A | C | 0.4021 | 0.5979 | 3.87E−05 | 0.94658 |
| 3 | rs3922844 | T | C | 0.3015 | 0.6985 | 8.95E−08 | 1.073474 |
| 3 | rs6795970 | A | G | 0.3995 | 0.6005 | 4.13E−06 | 0.94233 |
| 3 | rs6801957 | T | C | 0.4062 | 0.5938 | 1.56E−06 | 0.940541 |
| 3 | rs6800541 | C | T | 0.4062 | 0.5938 | 8.53E−07 | 0.938756 |
| 3 | rs2306272 | C | T | 0.2853 | 0.7147 | 2.69E−05 | 1.058338 |
| 3 | rs35124509 | C | T | 0.3943 | 0.6057 | 3.46E−05 | 0.947906 |
| 3 | rs7632427 | C | T | 0.4021 | 0.5979 | 2.96E−05 | 0.947622 |
| 3 | rs74505171 | G | A | 0.1529 | 0.8471 | 2.31E−05 | 0.924225 |
| 3 | rs16858949 | C | A | 0.1503 | 0.8497 | 4.95E−05 | 0.930903 |
| 3 | rs17220640 | C | T | 0.05448 | 0.94552 | 4.61E−05 | 1.168709 |
| 3 | rs16864142 | G | A | 0.1597 | 0.8403 | 1.91E−05 | 1.082313 |
| 4 | rs897945 | G | T | 0.4407 | 0.5593 | 2.87E−06 | 1.060775 |
| 4 | rs7691430 | T | G | 0.4405 | 0.5595 | 1.51E−06 | 1.06343 |
| 4 | rs34180226 | C | A | 0.1788 | 0.8212 | 4.29E−05 | 1.067586 |
| 4 | rs80285085 | T | C | 0.03681 | 0.96319 | 3.87E−05 | 1.220426 |
| 4 | rs78606958 | G | A | 0.03621 | 0.96379 | 6.16E−07 | 1.24309 |
| 4 | rs9997349 | G | A | 0.1997 | 0.8003 | 4.86E−06 | 1.076376 |
| 4 | rs1470618 | T | C | 0.1935 | 0.8065 | 2.78E−17 | 1.167191 |
| 4 | rs2595110 | G | A | 0.3228 | 0.6772 | 5.39E−09 | 0.917136 |
| 4 | rs2197815 | A | C | 0.05099 | 0.94901 | 3.21E−07 | 1.146599 |
| 4 | rs62339024 | G | A | 0.04521 | 0.95479 | 3.63E−06 | 0.786156 |
| 4 | rs13120244 | A | G | 0.1067 | 0.8933 | 3.31E−07 | 0.854448 |
| 4 | rs2723298 | T | C | 0.2763 | 0.7237 | 1.27E−57 | 1.24309 |
| 4 | rs1375302 | C | T | 0.2955 | 0.7045 | 1.55E−49 | 1.22544 |
| 4 | rs17625509 | G | A | 0.09958 | 0.90042 | 2.81E−06 | 0.875202 |
| 4 | rs1448817 | G | A | 0.2439 | 0.7561 | 3.92E−82 | 1.29693 |
| 4 | rs17042081 | T | G | 0.1047 | 0.8953 | 4.87E−130 | 1.513462 |
| 4 | rs78297632 | T | C | 0.04342 | 0.95658 | 2.53E−14 | 1.374926 |
| 4 | rs62337211 | A | C | 0.05091 | 0.94909 | 6.94E−06 | 0.807671 |
| 4 | rs2200733 | T | C | 0.1022 | 0.8978 | 2.32E−150 | 1.542493 |
| 4 | rs6843082 | G | A | 0.1931 | 0.8069 | 3.41E−155 | 1.448604 |
| 4 | rs11931959 | G | A | 0.2676 | 0.7324 | 1.70E−44 | 1.208403 |
| 4 | rs10033464 | T | G | 0.09091 | 0.90909 | 2.28E−15 | 1.162532 |
| 4 | rs13125644 | A | G | 0.09503 | 0.90497 | 1.36E−11 | 0.840885 |
| 4 | rs13141190 | A | G | 0.3526 | 0.6474 | 2.56E−78 | 1.270868 |
| 4 | rs4124163 | G | A | 0.05093 | 0.94907 | 3.35E−07 | 0.850356 |
| 4 | rs3853445 | C | T | 0.2629 | 0.7371 | 9.18E−31 | 0.847046 |
| 4 | rs13124249 | A | C | 0.3815 | 0.6185 | 3.62E−23 | 1.139512 |
| 4 | rs4033102 | G | A | 0.05232 | 0.94768 | 4.02E−08 | 0.861397 |
| 4 | rs521511 | T | C | 0.2039 | 0.7961 | 4.80E−13 | 0.893687 |
| 4 | rs489471 | T | C | 0.04893 | 0.95107 | 2.02E−10 | 0.828698 |
| 4 | rs512060 | G | A | 0.1447 | 0.8553 | 2.11E−05 | 0.926724 |
| 4 | rs17513625 | A | G | 0.02366 | 0.97634 | 2.47E−24 | 1.750147 |
| 4 | rs416532 | T | G | 0.39 | 0.61 | 1.12E−11 | 1.09823 |
| 4 | rs6823804 | A | G | 0.1319 | 0.8681 | 8.25E−06 | 0.925705 |
| 4 | rs13126426 | C | T | 0.04571 | 0.95429 | 4.76E−09 | 1.263897 |
| 4 | rs72672233 | A | G | 0.08616 | 0.91384 | 6.70E−07 | 1.112378 |
| 4 | rs297013 | G | A | 0.3579 | 0.6421 | 1.23E−07 | 1.070901 |
| 4 | rs159228 | C | T | 0.07898 | 0.92102 | 3.95E−07 | 1.109046 |
| 4 | rs17042914 | C | T | 0.1381 | 0.8619 | 1.92E−05 | 1.074763 |
| 5 | rs17410422 | T | C | 0.05293 | 0.94707 | 4.10E−05 | 1.188985 |
| 5 | rs337711 | T | C | 0.3936 | 0.6064 | 2.93E−08 | 1.072615 |
| 5 | rs11747175 | C | T | 0.2085 | 0.7915 | 1.53E−07 | 0.922839 |
| 5 | rs56335893 | A | G | 0.2071 | 0.7929 | 1.41E−07 | 0.920811 |
| 5 | rs4557401 | C | T | 0.1691 | 0.8309 | 1.00E−05 | 0.928765 |
| 5 | rs6596417 | T | C | 0.2131 | 0.7869 | 1.83E−06 | 0.930345 |
| 5 | rs2040862 | T | C | 0.1795 | 0.8205 | 1.11E−06 | 1.083829 |
| 5 | rs258757 | T | C | 0.1796 | 0.8204 | 6.48E−06 | 1.079286 |
| 5 | rs72802828 | T | C | 0.2425 | 0.7575 | 7.17E−06 | 1.070901 |
| 6 | rs618997 | G | T | 0.4433 | 0.5567 | 2.30E−05 | 1.067586 |
| 6 | rs59430691 | A | G | 0.1433 | 0.8567 | 7.26E−07 | 0.907465 |
| 6 | rs4716069 | C | T | 0.2471 | 0.7529 | 6.19E−06 | 0.933233 |
| 6 | rs214575 | C | T | 0.3662 | 0.6338 | 7.20E−06 | 1.059291 |
| 6 | rs9396839 | A | G | 0.2743 | 0.7257 | 1.86E−07 | 1.075408 |
| 6 | rs9367986 | A | C | 0.3731 | 0.6269 | 1.07E−05 | 1.058338 |
| 6 | rs11756403 | T | C | 0.2543 | 0.7457 | 1.63E−05 | 1.063324 |
| 6 | rs7756873 | C | T | 0.2864 | 0.7136 | 3.11E−06 | 1.065773 |
| 6 | rs201353 | G | A | 0.07424 | 0.92576 | 1.98E−06 | 1.1126 |
| 6 | rs4713191 | C | T | 0.0846 | 0.9154 | 5.51E−05 | 1.105281 |
| 6 | rs6456964 | T | C | 0.01787 | 0.98213 | 2.59E−06 | 0.801637 |
| 6 | rs9267659 | A | G | 0.2189 | 0.7811 | 5.50E−05 | 1.065986 |
| 6 | rs9470361 | A | G | 0.2351 | 0.7649 | 4.67E−06 | 0.935944 |
| 6 | rs762624 | C | A | 0.2601 | 0.7399 | 7.53E−06 | 0.93763 |
| 6 | rs6937605 | T | C | 0.1701 | 0.8299 | 2.61E−06 | 0.923486 |
| 6 | rs2196622 | G | A | 0.08447 | 0.91553 | 1.28E−05 | 1.096036 |
| 6 | rs281868 | A | G | 0.4972 | 0.5028 | 2.12E−09 | 0.928672 |
| 6 | rs11153730 | C | T | 0.494 | 0.506 | 2.01E−05 | 0.948759 |
| 6 | rs11970286 | T | C | 0.4552 | 0.5448 | 3.29E−05 | 0.949519 |
| 6 | rs25422 | T | C | 0.1774 | 0.8226 | 5.70E−05 | 1.064601 |
| 6 | rs62422230 | C | T | 0.1797 | 0.8203 | 8.49E−06 | 1.074978 |
| 6 | rs868153 | G | T | 0.3559 | 0.6441 | 4.01E−07 | 0.935663 |
| 6 | rs2066121 | G | T | 0.3024 | 0.6976 | 4.29E−07 | 0.93342 |
| 6 | rs7761904 | C | T | 0.2926 | 0.7074 | 1.04E−05 | 0.940447 |
| 6 | rs12524865 | A | C | 0.2895 | 0.7105 | 6.74E−07 | 1.069723 |
| 6 | rs6557317 | T | C | 0.4849 | 0.5151 | 2.99E−05 | 1.052954 |
| 7 | rs10254657 | A | G | 0.4287 | 0.5713 | 8.10E−07 | 1.066732 |
| 7 | rs7782982 | G | T | 0.2496 | 0.7504 | 7.11E−06 | 0.932674 |
| 7 | rs79215950 | A | G | 0.388 | 0.612 | 1.45E−05 | 1.067693 |
| 7 | rs2215614 | C | A | 0.3872 | 0.6128 | 8.56E−06 | 1.061518 |
| 7 | rs28495309 | A | G | 0.3018 | 0.6982 | 1.20E−05 | 1.072401 |
| 7 | rs13244544 | A | G | 0.07989 | 0.92011 | 7.99E−06 | 1.106719 |
| 7 | rs10277880 | A | G | 0.1383 | 0.8617 | 1.05E−05 | 1.078963 |
| 7 | rs17156851 | C | T | 0.222 | 0.778 | 3.01E−05 | 1.065027 |
| 7 | rs74301815 | T | C | 0.06035 | 0.93965 | 1.11E−05 | 1.130884 |
| 7 | rs2402056 | A | C | 0.4215 | 0.5785 | 2.26E−05 | 0.947432 |
| 7 | rs760444 | G | A | 0.1819 | 0.8181 | 1.56E−06 | 0.926816 |
| 7 | rs10235210 | C | T | 0.4177 | 0.5823 | 3.09E−06 | 0.94365 |
| 7 | rs1728723 | G | T | 0.2721 | 0.7279 | 5.53E−07 | 0.933513 |
| 7 | rs6976316 | G | A | 0.4834 | 0.5166 | 1.60E−12 | 0.917136 |
| 7 | rs3807988 | C | A | 0.07509 | 0.92491 | 3.65E−05 | 0.909191 |
| 7 | rs11773845 | C | A | 0.4091 | 0.5909 | 7.53E−15 | 0.907647 |
| 7 | rs2109517 | G | A | 0.3304 | 0.6696 | 4.21E−07 | 0.935663 |
| 7 | rs6955302 | T | C | 0.3216 | 0.6784 | 3.39E−06 | 0.93885 |
| 8 | rs17515291 | G | A | 0.2625 | 0.7375 | 1.82E−06 | 0.933607 |
| 8 | rs2306643 | G | T | 0.1135 | 0.8865 | 5.69E−05 | 0.92626 |
| 8 | rs2719008 | C | T | 0.468 | 0.532 | 2.73E−05 | 0.949139 |
| 8 | rs62521286 | G | A | 0.06688 | 0.93312 | 1.25E−06 | 1.13519 |
| 8 | rs10104625 | G | A | 0.1699 | 0.8301 | 1.96E−05 | 1.071651 |
| 8 | rs7814359 | G | A | 0.2148 | 0.7852 | 3.77E−05 | 1.064494 |
| 8 | rs4520288 | T | C | 0.2107 | 0.7893 | 4.41E−05 | 1.064388 |
| 9 | rs10809847 | C | T | 0.2441 | 0.7559 | 3.54E−05 | 0.940541 |
| 9 | rs446540 | A | G | 0.4055 | 0.5945 | 3.29E−11 | 1.087194 |
| 9 | rs10821415 | A | C | 0.4166 | 0.5834 | 6.08E−11 | 1.086107 |
| 9 | rs6537893 | A | G | 0.09214 | 0.90786 | 5.42E−05 | 1.091988 |
| 9 | rs7860634 | G | A | 0.4245 | 0.5755 | 1.57E−05 | 0.943744 |
| 9 | rs7031621 | G | A | 0.4965 | 0.5035 | 4.77E−05 | 0.947527 |
| 10 | rs74967172 | C | T | 0.03083 | 0.96917 | 3.33E−05 | 1.271885 |
| 10 | rs60632610 | T | C | 0.1402 | 0.8598 | 2.30E−10 | 0.891099 |
| 10 | rs12570126 | G | A | 0.1469 | 0.8531 | 1.28E−08 | 0.91092 |
| 10 | rs2306325 | C | T | 0.1318 | 0.8682 | 9.51E−08 | 0.909373 |
| 10 | rs2242254 | A | G | 0.1375 | 0.8625 | 1.20E−07 | 0.908373 |
| 10 | rs704015 | A | C | 0.1716 | 0.8284 | 2.07E−05 | 1.074655 |
| 10 | rs9651450 | T | C | 0.2956 | 0.7044 | 4.74E−05 | 0.945256 |
| 10 | rs7083450 | C | T | 0.1551 | 0.8449 | 1.76E−05 | 1.074655 |
| 10 | rs6584555 | C | T | 0.151 | 0.849 | 2.10E−19 | 1.154538 |
| 10 | rs55706621 | T | C | 0.3941 | 0.6059 | 9.36E−10 | 1.081663 |
| 10 | rs1018014 | G | A | 0.2321 | 0.7679 | 1.78E−11 | 0.90303 |
| 10 | rs12772784 | T | C | 0.1394 | 0.8606 | 9.59E−07 | 1.091661 |
| 10 | rs879544 | C | T | 0.235 | 0.765 | 7.22E−07 | 1.073581 |
| 10 | rs11592832 | T | C | 0.1116 | 0.8884 | 1.63E−09 | 1.122435 |
| 10 | rs2236280 | G | T | 0.1761 | 0.8239 | 2.42E−08 | 1.093627 |
| 10 | rs7092718 | C | T | 0.2368 | 0.7632 | 6.99E−07 | 1.071972 |
| 10 | rs35623220 | T | C | 0.1066 | 0.8934 | 2.76E−07 | 1.111711 |
| 10 | rs2487999 | T | C | 0.09571 | 0.90429 | 1.80E−07 | 1.107273 |
| 10 | rs9420907 | C | A | 0.1307 | 0.8693 | 4.59E−05 | 1.074548 |
| 11 | rs7106847 | A | G | 0.2012 | 0.7988 | 5.02E−05 | 1.065666 |
| 11 | rs10741807 | T | C | 0.2239 | 0.7761 | 5.59E−06 | 1.068547 |
| 11 | rs74902063 | A | G | 0.03595 | 0.96405 | 5.32E−05 | 0.737566 |
| 11 | rs543389 | A | G | 0.1195 | 0.8805 | 1.10E−05 | 1.08513 |
| 11 | rs76097649 | A | G | 0.09007 | 0.90993 | 1.96E−08 | 1.171752 |
| 11 | rs2604192 | A | G | 0.188 | 0.812 | 1.95E−05 | 1.070365 |
| 11 | rs57662446 | A | G | 0.1814 | 0.8186 | 3.22E−05 | 1.070901 |
| 12 | rs11062287 | A | G | 0.04231 | 0.95769 | 3.24E−05 | 0.69385 |
| 12 | rs17287293 | G | A | 0.1503 | 0.8497 | 1.84E−07 | 0.909282 |
| 12 | rs448508 | T | C | 0.216 | 0.784 | 2.34E−05 | 1.080474 |
| 12 | rs17782296 | A | G | 0.1327 | 0.8673 | 3.92E−05 | 1.083287 |
| 12 | rs10506569 | T | C | 0.467 | 0.533 | 1.04E−05 | 0.94535 |
| 12 | rs1895596 | A | G | 0.1603 | 0.8397 | 6.12E−09 | 0.903301 |
| 12 | rs883079 | C | T | 0.2648 | 0.7352 | 1.80E−15 | 0.897269 |
| 12 | rs3825214 | G | A | 0.1857 | 0.8143 | 1.82E−10 | 0.906558 |
| 12 | rs7310159 | C | T | 0.2697 | 0.7303 | 3.47E−06 | 1.068013 |
| 12 | rs1895585 | A | G | 0.2453 | 0.7547 | 1.25E−14 | 0.897179 |
| 13 | rs12430408 | C | T | 0.02326 | 0.97674 | 4.33E−05 | 1.249321 |
| 13 | rs275945 | G | A | 0.3496 | 0.6504 | 5.04E−05 | 1.053165 |
| 14 | rs7621 | G | T | 0.2987 | 0.7013 | 5.37E−05 | 1.055062 |
| 14 | rs7161192 | A | C | 0.3138 | 0.6862 | 3.57E−07 | 0.935102 |
| 14 | rs1255986 | T | C | 0.4977 | 0.5023 | 7.00E−10 | 0.925797 |
| 14 | rs7148072 | A | G | 0.3341 | 0.6659 | 2.31E−07 | 0.933513 |
| 14 | rs1152590 | T | C | 0.3666 | 0.6334 | 1.76E−07 | 0.934447 |
| 14 | rs1269056 | T | C | 0.4446 | 0.5554 | 1.25E−07 | 1.068761 |
| 14 | rs2987971 | A | G | 0.4316 | 0.5684 | 5.49E−05 | 0.950279 |
| 14 | rs178348 | C | T | 0.3533 | 0.6467 | 4.19E−05 | 0.948759 |
| 14 | rs221482 | G | T | 0.1986 | 0.8014 | 1.70E−05 | 0.936037 |
| 15 | rs276857 | G | A | 0.1363 | 0.8637 | 1.62E−06 | 1.098779 |
| 15 | rs11636462 | G | T | 0.07245 | 0.92755 | 1.99E−06 | 1.120192 |
| 15 | rs7164883 | G | A | 0.1572 | 0.8428 | 6.85E−11 | 1.109379 |
| 15 | rs56390326 | A | G | 0.08532 | 0.91468 | 1.31E−06 | 1.113602 |
| 16 | rs13336114 | A | C | 0.3312 | 0.6688 | 3.26E−05 | 1.06343 |
| 16 | rs7193343 | T | C | 0.1621 | 0.8379 | 2.48E−19 | 1.152231 |
| 16 | rs719353 | A | G | 0.4459 | 0.5541 | 5.30E−10 | 1.081447 |
| 16 | rs62053234 | T | G | 0.2045 | 0.7955 | 7.62E−07 | 0.919523 |
| 16 | rs2106261 | T | C | 0.1686 | 0.8314 | 8.18E−32 | 1.202617 |
| 16 | rs879324 | A | G | 0.17 | 0.83 | 1.13E−25 | 1.180927 |
| 16 | rs9940321 | A | G | 0.3464 | 0.6536 | 5.41E−14 | 1.113602 |
| 16 | rs61164185 | A | G | 0.2075 | 0.7925 | 2.11E−09 | 1.100429 |
| 16 | rs9930504 | T | C | 0.2663 | 0.7337 | 1.59E−07 | 1.090788 |
| 16 | rs1183967 | T | C | 0.1737 | 0.8263 | 1.27E−07 | 1.112378 |
| 17 | rs7224248 | C | T | 0.04836 | 0.95164 | 1.30E−05 | 0.883733 |
| 17 | rs34003767 | G | A | 0.3697 | 0.6303 | 2.56E−07 | 0.934541 |
| 17 | rs7502233 | G | A | 0.3358 | 0.6642 | 4.21E−06 | 0.938193 |
| 19 | rs57893103 | G | A | 0.1037 | 0.8963 | 5.23E−05 | 0.915395 |
| 19 | rs118182626 | A | C | 0.03681 | 0.96319 | 1.18E−05 | 0.726585 |
| 20 | rs55776240 | A | G | 0.1304 | 0.8696 | 5.53E−05 | 0.929601 |
| 21 | rs9978265 | A | G | 0.08559 | 0.91441 | 4.08E−05 | 1.112155 |
| 22 | rs361540 | G | A | 0.4159 | 0.5841 | 8.52E−06 | 0.94167 |
| 22 | rs5752233 | G | A | 0.3808 | 0.6192 | 1.24E−05 | 1.058232 |
| 22 | rs695351 | G | A | 0.2682 | 0.7318 | 4.80E−06 | 1.068868 |

The present invention further provides a method for assessing the risk of a human subject developing Type 2 diabetes, the method comprising performing a genetic risk assessment of the human subject, wherein the genetic risk assessment involves detecting, in a biological sample derived from the human subject, the presence at least one polymorphisms associated with a risk of developing Type 2 diabetes, wherein the at least one polymorphism is selected from Table 4, or a single nucleotide polymorphism in linkage disequilibrium with one or more thereof. In an embodiment, the method comprises detecting the presence of at least two, at least three, at least four, at least five, at least six, at least seven, at least eight, at least nine, at least ten, at least 20, at least 30, at least 40, at least 50, at least 60, at least 70, at least 80, at least 85, or all of the polymorphism provided in Table 4, or a single nucleotide polymorphism in linkage disequilibrium with one or more thereof. In an embodiment, the method comprises detecting each of the polymorphism provided in Table 4.

TABLE 4
Polymorphisms associated with Type 2 diabetes. A1 is the risk allele.

| CHR | SNP | A1 | A2 | A1_freq | A2_freq | p_value | OR |
|---|---|---|---|---|---|---|---|
| 1 | rs16826069 | G | A | 0.2144 | 0.7856 | 2.20E−06 | 1.071436 |
| 1 | rs340874 | T | C | 0.4274 | 0.5726 | 3.40E−08 | 0.93426 |
| 1 | rs4846567 | T | G | 0.3001 | 0.6999 | 2.60E−08 | 0.929601 |
| 2 | rs11124935 | C | T | 0.3896 | 0.6104 | 2.20E−06 | 0.941765 |
| 2 | rs7578597 | C | T | 0.109 | 0.891 | 2.10E−10 | 0.878095 |
| 2 | rs9309325 | G | A | 0.4559 | 0.5441 | 2.80E−08 | 0.93426 |
| 2 | rs13412977 | C | T | 0.4784 | 0.5216 | 8.70E−07 | 1.061837 |
| 2 | rs2972143 | A | G | 0.3524 | 0.6476 | 8.20E−09 | 0.930531 |
| 3 | rs17036328 | C | T | 0.1211 | 0.8789 | 5.50E−12 | 0.878095 |
| 3 | rs76871646 | C | T | 0.02424 | 0.97576 | 6.20E−07 | 0.852144 |
| 3 | rs6795735 | T | C | 0.4052 | 0.5948 | 2.10E−08 | 0.931462 |
| 3 | rs11708067 | G | A | 0.2453 | 0.7547 | 8.80E−13 | 0.895834 |
| 3 | rs4402960 | T | G | 0.3136 | 0.6864 | 2.70E−25 | 1.150274 |
| 3 | rs77494444 | T | C | 0.05236 | 0.94764 | 2.00E−09 | 1.197217 |
| 4 | rs734312 | G | A | 0.4564 | 0.5436 | 1.80E−14 | 0.910283 |
| 4 | rs1509941 | C | T | 0.4748 | 0.5252 | 3.60E−06 | 0.945539 |
| 4 | rs11097755 | C | T | 0.4427 | 0.5573 | 1.60E−06 | 1.065027 |
| 4 | rs72679551 | G | A | 0.05114 | 0.94886 | 2.30E−06 | 1.127497 |
| 4 | rs11936062 | T | G | 0.1618 | 0.8382 | 3.30E−07 | 0.918512 |
| 5 | rs3212656 | C | T | 0.2086 | 0.7914 | 3.10E−06 | 0.933327 |
| 5 | rs9687846 | A | G | 0.2007 | 0.7993 | 2.80E−09 | 1.099659 |
| 6 | rs 76393458 | A | G | 0.01818 | 0.98182 | 2.80E−07 | 1.246077 |
| 6 | rs7756992 | G | A | 0.2654 | 0.7346 | 2.30E−34 | 1.185305 |
| 6 | rs9350276 | T | C | 0.3949 | 0.6051 | 6.80E−09 | 1.073581 |
| 6 | rs60568948 | C | T | 0.08293 | 0.91707 | 2.10E−08 | 1.127497 |
| 6 | rs3131012 | T | C | 0.4542 | 0.5458 | 8.10E−07 | 0.939883 |
| 6 | rs3129768 | G | T | 0.1804 | 0.8196 | 4.00E−07 | 0.914846 |
| 6 | rs9388489 | G | A | 0.4498 | 0.5502 | 2.10E−08 | 1.071436 |
| 7 | rs12530627 | A | G | 0.4802 | 0.5198 | 4.00E−07 | 0.938005 |
| 7 | rs10276674 | C | T | 0.1808 | 0.8192 | 5.10E−08 | 1.088717 |
| 7 | rs10244051 | T | G | 0.4516 | 0.5484 | 2.40E−08 | 0.93426 |
| 7 | rs1635852 | T | C | 0.4935 | 0.5065 | 3.00E−14 | 1.096365 |
| 8 | rs516946 | T | C | 0.2372 | 0.7628 | 8.60E−07 | 0.929601 |
| 8 | rs2464592 | G | A | 0.2832 | 0.7168 | 1.20E−06 | 1.068227 |
| 8 | rs3802177 | A | G | 0.31 | 0.69 | 1.70E−17 | 0.895834 |
| 8 | rs951337 | T | C | 0.4803 | 0.5197 | 4.00E−09 | 0.930531 |
| 8 | rs3757972 | T | C | 0.3799 | 0.6201 | 3.10E−08 | 1.077884 |
| 9 | rs117626887 | G | A | 0.02213 | 0.97787 | 1.10E−07 | 1.20925 |
| 9 | rs4977756 | G | A | 0.4042 | 0.5958 | 2.30E−09 | 0.929601 |
| 9 | rs10965250 | A | G | 0.1712 | 0.8288 | 2.70E−17 | 0.869358 |
| 9 | rs7018475 | G | T | 0.255 | 0.745 | 7.20E−16 | 1.127497 |
| 9 | rs2796441 | A | G | 0.4211 | 0.5789 | 5.50E−08 | 0.930531 |
| 9 | rs507666 | A | G | 0.1845 | 0.8155 | 5.50E−07 | 1.083287 |
| 10 | rs11257655 | T | C | 0.2091 | 0.7909 | 4.00E−08 | 1.083287 |
| 10 | rs12571751 | G | A | 0.4612 | 0.5388 | 1.30E−12 | 0.915761 |
| 10 | rs7903767 | G | A | 0.467 | 0.533 | 9.10E−07 | 1.061837 |
| 10 | rs11187065 | C | T | 0.2135 | 0.7865 | 7.50E−08 | 1.083287 |
| 10 | rs1111875 | T | C | 0.4085 | 0.5915 | 3.60E−25 | 0.878095 |
| 10 | rs4933236 | C | T | 0.3894 | 0.6106 | 2.10E−07 | 0.93426 |
| 10 | rs7917983 | C | T | 0.4656 | 0.5344 | 1.50E−18 | 1.116278 |
| 10 | rs11196181 | A | G | 0.06053 | 0.93947 | 1.60E−07 | 0.869358 |
| 10 | rs75933965 | A | G | 0.0691 | 0.9309 | 6.60E−18 | 1.246077 |
| 10 | rs116859590 | T | C | 0.02625 | 0.97375 | 2.90E−10 | 1.336427 |
| 10 | rs7903146 | T | C | 0.2894 | 0.7106 | 9.30E−108 | 1.336427 |
| 10 | rs74825300 | T | C | 0.09144 | 0.90856 | 1.20E−07 | 0.878095 |
| 10 | rs116369954 | C | T | 0.03151 | 0.96849 | 1.60E−17 | 1.349859 |
| 10 | rs61872787 | G | A | 0.02321 | 0.97679 | 9.00E−11 | 1.433329 |
| 10 | rs4918788 | A | G | 0.4619 | 0.5381 | 3.30E−11 | 1.084371 |
| 10 | rs6585827 | A | G | 0.4709 | 0.5291 | 2.20E−12 | 0.918512 |
| 11 | rs231364 | A | G | 0.08436 | 0.91564 | 1.30E−06 | 1.116278 |
| 11 | rs231362 | A | G | 0.4759 | 0.5241 | 1.90E−09 | 0.92589 |
| 11 | rs163168 | T | C | 0.22 | 0.78 | 5.50E−07 | 0.924964 |
| 11 | rs2237892 | T | C | 0.06402 | 0.93598 | 1.10E−07 | 0.878095 |
| 11 | rs2237895 | C | A | 0.4147 | 0.5853 | 1.70E−13 | 1.10186 |
| 11 | rs5219 | T | C | 0.3584 | 0.6416 | 4.30E−08 | 1.070365 |
| 11 | rs1061810 | A | C | 0.2914 | 0.7086 | 5.30E−09 | 1.083287 |
| 11 | rs10791821 | A | G | 0.2659 | 0.7341 | 2.70E−06 | 1.068227 |
| 11 | rs11603334 | A | G | 0.1561 | 0.8439 | 5.30E−09 | 0.909373 |
| 12 | rs11063018 | C | T | 0.1717 | 0.8283 | 1.30E−07 | 1.091988 |
| 12 | rs10842994 | T | C | 0.1953 | 0.8047 | 7.10E−07 | 0.92404 |
| 12 | rs2686552 | T | C | 0.4124 | 0.5876 | 3.60E−07 | 0.938005 |
| 13 | rs1359790 | A | G | 0.2891 | 0.7109 | 1.40E−08 | 0.923116 |
| 15 | rs7173964 | A | G | 0.4281 | 0.5719 | 8.20E−08 | 0.937067 |
| 15 | rs7178572 | A | G | 0.2869 | 0.7131 | 4.40E−10 | 0.921272 |
| 15 | rs8042680 | A | C | 0.3005 | 0.6995 | 1.20E−06 | 1.067159 |
| 16 | rs12149832 | A | G | 0.4122 | 0.5878 | 3.70E−24 | 1.127497 |
| 16 | rs72802340 | T | G | 0.03811 | 0.96189 | 1.60E−06 | 0.818731 |
| 16 | rs2925979 | T | C | 0.302 | 0.698 | 2.70E−08 | 1.076807 |
| 17 | rs12602456 | T | C | 0.238 | 0.762 | 1.60E−06 | 1.073581 |
| 17 | rs17681684 | A | G | 0.3319 | 0.6681 | 3.10E−07 | 1.070365 |
| 17 | rs12601991 | T | G | 0.4063 | 0.5937 | 2.00E−09 | 0.922194 |
| 17 | rs1451506 | A | G | 0.1157 | 0.8843 | 1.30E−06 | 1.105171 |
| 18 | rs7234111 | C | T | 0.3831 | 0.6169 | 7.70E−07 | 1.065027 |
| 18 | rs35981845 | G | A | 0.3354 | 0.6646 | 2.60E−07 | 1.067158 |
| 18 | rs11152214 | C | A | 0.1272 | 0.8728 | 1.70E−06 | 0.911194 |
| 19 | rs4420638 | G | A | 0.1907 | 0.8093 | 1.50E−09 | 0.895834 |
| 19 | rs10415769 | T | G | 0.2758 | 0.7242 | 3.50E−06 | 1.065027 |
| 19 | rs8111071 | G | A | 0.08168 | 0.91832 | 5.60E−07 | 1.127497 |
| 22 | rs73883360 | A | G | 0.09306 | 0.90694 | 1.40E−07 | 0.88692 |
| 22 | rs713669 | C | T | 0.4811 | 0.5189 | 2.40E−06 | 0.941765 |

As the skilled addressee will appreciate, each SNP which increases the risk of developing coronary artery disease, atrial fibrillation or Type 2 diabetes has an odds ratio of association with coronary artery disease, atrial fibrillation or Type 2 diabetes, respectively, of greater than 1.0. Furthermore, each SNP which decreases the risk of developing coronary artery disease, atrial fibrillation or Type 2 diabetes has an odds ratio of association with coronary artery disease, atrial fibrillation or Type 2 diabetes, respectively, of less than 1.0. In an embodiment, none of the polymorphisms have an odds ratio of association with coronary artery disease, atrial fibrillation or Type 2 diabetes greater than 3 or greater than 4.

In an example, single nucleotide polymorphisms in linkage disequilibrium with one or more of the single nucleotide polymorphisms selected from Table 1, Table 2, Table 3 or Table 4 have LD values of at least 0.5, at least 0.6, at least 0.7, at least 0.8. In another example, single nucleotide polymorphisms in linkage disequilibrium have LD values of at least 0.9. In another example, single nucleotide polymorphisms in linkage disequilibrium have LD values of at least 1.

In an embodiment, the number of SNPs assessed is based on the net reclassification improvement in risk prediction calculated using net reclassification index (NRI) (Pencina et al., 2008). In an embodiment, the net reclassification improvement of the methods of the present disclosure is greater than 0.01.

In a further embodiment, the net reclassification improvement of the methods of the present disclosure is greater than 0.05. In yet another embodiment, the net reclassification improvement of the methods of the present disclosure is greater than 0.1.

In an additional embodiment, variants in linkage disequilibrium with those specifically mentioned herein are easily identified by those of skill in the art. Variants that exist in strong linkage disequilibrium with those specifically mentioned herein, such as variants that are linked with an r2>0.8 and D′>0.8 would also be considered as existing in positive linkage disequilibrium sufficient to be considered a surrogate or proxy marker variant for the variants specifically described here. Individuals skilled in the art will be able to assess the inheritance.

### Calculating Composite SNP Relative Risk “Genetic Risk”

An individual's “genetic risk” can be defined as the weighted sum of the individuals' genotypes at multiple genetic loci. In other words, they are the linear combinations of the risk alleles across a set of candidate SNPs. For example, a PRS for individual i can be calculated as:

PRSᵢ=βᵢxᵢ₁+β₂xᵢ₂+ . . . +βⱼxᵢⱼ+ . . . +βₚxᵢₚ, (1)

where xᵢⱼ∈{0, 1, 2} are risk allele counts and βᵢⱼ are the weights for SNP j=1, . . . , p.

The PRS can also be represented as: PRS=(No of Risk Alleles×(3 coefficient).

In one embodiment, the key steps to construct a polygenic risk score (PRS) are to determine which SNPs to include and how to weight their effects. In one embodiment, the maxCT and SCT methods (Prive et al., 2019) are used. These methods are based on clumping and thresholding. The aim of clumping and thresholding is to remove correlated SNPs while keeping the most important SNPs in the PRS. In order to do this, the values of a range of hyperparameters including the correlation threshold (r2), the clumping window size (kb) and the p-value significance threshold (p) are decided. Different selection of these hyperparameters values would in general give a different section of SNPs to include. For the weights, the reported GWAS coefficients (i.e., regression coefficients or log odds ratios) from external published GWAS can be used.

The core idea of the maxCT and SCT procedures is to select a set of different hyperparameters values and compute a PRS for each combination of these values.

This will usually create a large number of PRSs, for example, around 100,000 vectors of PRSs for a typical GWAS. After constructing these RPSs, there are two approaches to create the final PRS. In maxCT, the PRS that has the strongest predictive performance (e.g., largest AUC) are selected as the final PRS. In SCT, the PRSs by a penalized logistic regression model, for example, the popular lasso procedure are combined. Since the outcome of SCT will be a linear combination of PRSs, where each PRS is again a linear combination of variants, the final PRS still has the form of (1), which means the effect sizes of SNPs can be obtained and used for prediction.

In an alternate example, a log-additive risk model can then be used to define three genotypes AA, AB, and BB for a single SNP having relative risk values of 1, OR, and OR², under a rare disease model, where OR is the previously reported disease odds ratio for the high-risk allele, B, vs the low-risk allele, A. If the B allele has frequency (p), then these genotypes have population frequencies of (1−p)², 2p(1−p), and p², assuming Hardy-Weinberg equilibrium. The genotype relative risk values for each SNP can then be scaled so that based on these frequencies the average relative risk in the population is 1. Specifically, given the unscaled population average relative risk:

(μ)=(1−p)²+2p(1−p)OR+p²OR²

Adjusted risk values 1/μ, OR/μ, and OR²/μ are used for AA, AB, and BB genotypes. Missing genotypes are assigned a relative risk of 1. The following formula can be used to define the genetic risk:

SNP₁×SNP₂×SNP₃×SNP₄×SNP₅×SNP₆×SNP₇,×SNP₈, etc.

Similar calculations can be performed for non-SNP polymorphisms.

An alternate method for calculating the composite SNP risk is described in Mavaddat et al. (2015). In this example, the following formula is used;

PRS=β₁x₁+β_{b}x₂+ . . . β_{κ}x_{κ}+βₙxₙ

where β_{κ} is the per-allele log odds ratio (OR) for coronary artery disease, atrial fibrillation or Type 2 diabetes associated with the minor allele for SNP κ, and x_{κ} the number of alleles for the same SNP (0, 1 or 2), n is the total number of SNPs and PRS is the polygenic risk score (which can also be referred to as composite SNP risk).

It is envisaged that the “risk” of a human subject for developing coronary artery disease, atrial fibrillation or Type 2 diabetes can be provided as a relative risk (or risk ratio) or an absolute risk as required.

In an embodiment, the genetic risk assessment obtains the “relative risk” of a human subject for developing coronary artery disease, atrial fibrillation or Type 2 diabetes. Relative risk (or risk ratio), measured as the incidence of a disease in individuals with a particular characteristic (or exposure) divided by the incidence of the disease in individuals without the characteristic, indicates whether that particular exposure increases or decreases risk. Relative risk is helpful to identify characteristics that are associated with a disease, but by itself is not particularly helpful in guiding screening decisions because the frequency of the risk (incidence) is cancelled out.

In another embodiment, the genetic risk assessment obtains the “absolute risk” of a human subject for developing coronary artery disease, atrial fibrillation or Type 2 diabetes. Absolute risk is the numerical probability of a human subject developing coronary artery disease, atrial fibrillation or Type 2 diabetes within a specified period (e.g. 5, 10, 15, 20 or more years). It reflects a human subject's risk of developing coronary artery disease, atrial fibrillation or Type 2 diabetes in so far as it does not consider various risk factors in isolation.

In an embodiment, one or more threshold value(s) are set for determining a particular action such as the need for routine diagnostic testing or preventative therapy. For example, a score determined using a method of the invention is compared to a pre-determined threshold, and if the score is higher than the threshold a recommendation is made to take the pre-determined action. Methods of setting such thresholds have now become widely used in the art and are described in, for example, US 20140018258.

### Clinical Risk Assessment

The methods of the present disclosure can comprise performing a clinical risk assessment of the subject. The results of the clinical risk assessment can be combined with the genetic risk assessment to obtain the risk of the subject for developing coronary artery disease, atrial fibrillation or Type 2 diabetes.

Any suitable clinical risk assessment procedure can be used in the present disclosure. Preferably, the clinical risk assessment does not involve genotyping the subject at one or more loci.

In one embodiment, the clinical risk assessment procedure includes obtaining information from the subject on one or more of the following: age, gender, HDL-cholesterol level (mmol/L), LDL-cholesterol level (mmol/L), total cholesterol level, blood pressure (systolic and/or diastolic (mm Hg)), smoking status, have or has had diabetes, on hypertension medication, c-reactive protein levels, whether the subject's mother or father have had a heart attack (such by the age of 60), body mass index, ethnicity, measures of deprivation, family history, have or has had chronic kidney disease, and have or has had rheumatoid arthritis.

In another embodiment, the clinical risk assessment procedure includes obtaining information from the subject on one or more of the following: age, gender, HDL-cholesterol level (mmol/L), LDL-cholesterol level (mmol/L), total cholesterol level, blood pressure (systolic and diastolic (mm Hg)), smoking status, have or has had diabetes and on hypertension medication.

In another embodiment, performing the clinical risk assessment uses a model which calculates the absolute risk of developing coronary artery disease, atrial fibrillation or Type 2 diabetes. For example, the absolute risk of developing coronary artery disease, atrial fibrillation or Type 2 diabetes can be calculated using coronary artery disease, atrial fibrillation or Type 2 diabetes, respectively, incidence rates while accounting for the competing risk of dying from other causes apart from coronary artery disease, atrial fibrillation or Type 2 diabetes. In an embodiment, the clinical risk assessment provides a 5-year absolute risk of developing coronary artery disease, atrial fibrillation or Type 2 diabetes. In another embodiment, the clinical risk assessment provides a 10-year absolute risk of developing coronary artery disease, atrial fibrillation or Type 2 diabetes.

Examples of clinical risk assessment procedures include, but are not limited to, the Framingham score, the Reynolds score, QRISK and CANRISK (for diabetes). In a preferred embodiment, the clinical risk assessment is the Framingham Risk Score for the subject. In another preferred embodiment, the clinical risk assessment is The American College of Cardiologists Pooled Cohort Equations (PCE). In a preferred embodiment, the Framingham score is used to determine the risk of developing Atrial Fibrillation or Type 2 Diabetes. In a preferred embodiment, PCE is used to determine the risk of developing Coronary Artery Disease.

The Framingham Risk Score is an algorithm used to estimate the 10-year cardiovascular risk of an individual. The Framingham Risk Score was first developed based on data obtained from the Framingham Heart Study, to estimate the 10-year risk of developing coronary heart disease (Wilson et al., 1998). In order to assess the 10-year cardiovascular disease risk, cerebrovascular events, peripheral artery disease and heart failure were subsequently added as disease outcomes for the 2008 Framingham Risk Score, on top of coronary heart disease (D'Agostino et al., 2008). The Framingham Risk Score has also been shown to be a useful predictor of developing atrial fibrillation and Type 2 diabetes.

In an embodiment, for a female subject the Framingham Risk Score is determined as follows:

Age: 20-34 years: Minus 7 points. 35-39 years: Minus 3 points. 40-44 years: 0 points. 45-49 years: 3 points. 50-54 years: 6 points. 55-59 years: 8 points. 60-64 years: points. 65-69 years: 12 points. 70-74 years: 14 points. 75-79 years: 16 points.

Total cholesterol, mg/dL: Age 20-39 years: Under 160: 0 points. 160-199: 4 points. 200-239: 8 points. 240-279: 11 points. 280 or higher: 13 points. ⋅ Age 40-49 years: Under 160: 0 points. 160-199: 3 points. 200-239: 6 points. 240-279: 8 points. 280 or higher: 10 points. ⋅ Age 50-59 years: Under 160: 0 points. 160-199: 2 points. 200-239: 4 points. 240-279: 5 points. 280 or higher: 7 points. ⋅ Age 60-69 years: Under 160: points. 160-199: 1 point. 200-239: 2 points. 240-279: 3 points. 280 or higher: 4 points. ⋅ Age 70-79 years: Under 160: 0 points. 160-199: 1 point. 200-239: 1 point. 240-279: 2 points. 280 or higher: 2 points.

If cigarette smoker: Age 20-39 years: 9 points. ⋅ Age 40-49 years: 7 points. ⋅ Age years: 4 points. ⋅ Age 60-69 years: 2 points. ⋅ Age 70-79 years: 1 point.

All non smokers: 0 points.

HDL cholesterol, mg/dL: 60 or higher: Minus 1 point. 50-59: 0 points. 40-49: 1 point. Under 40: 2 points.

Systolic blood pressure, mm Hg: Untreated: Under 120: 0 points. 120-129: 1 point. 130-139: 2 points. 140-159: 3 points. 160 or higher: 4 points. ⋅ Treated: Under 120: 0 points. 120-129: 3 points. 130-139: 4 points. 140-159: 5 points. 160 or higher: 6 points.

10-year risk in %: Points total: Under 9 points: <1%. 9-12 points: 1%. 13-14 points: 2%. 15 points: 3%. 16 points: 4%. 17 points: 5%. 18 points: 6%. 19 points: 8%. 20 points: 11%. 21=14%, 22=17%, 23=22%, 24=27%, >25=Over 30%

In an embodiment, for a male subject the Framingham Risk Score is determined as follows:

Age: 20-34 years: Minus 9 points. 35-39 years: Minus 4 points. 40-44 years: 0 points. 45-49 years: 3 points. 50-54 years: 6 points. 55-59 years: 8 points. 60-64 years: 10 points. 65-69 years: 11 points. 70-74 years: 12 points. 75-79 years: 13 points.

Total cholesterol, mg/dL: Age 20-39 years: Under 160: 0 points. 160-199: 4 points. 200-239: 7 points. 240-279: 9 points. 280 or higher: 11 points. ⋅ Age 40-49 years: Under 160: 0 points. 160-199: 3 points. 200-239: 5 points. 240-279: 6 points. 280 or higher: 8 points. ⋅ Age 50-59 years: Under 160: 0 points. 160-199: 2 points. 200-239: 3 points. 240-279: 4 points. 280 or higher: 5 points. ⋅ Age 60-69 years: Under 160: 0 points. 160-199: 1 point. 200-239: 1 point. 240-279: 2 points. 280 or higher: 3 points. ⋅ Age 70-79 years: Under 160: 0 points. 160-199: 0 points. 200-239: 0 points. 240-279: 1 point. 280 or higher: 1 point.

If cigarette smoker: Age 20-39 years: 8 points. Age 40-49 years: 5 points. ⋅ Age 50-59 years: 3 points. ⋅ Age 60-69 years: 1 point. ⋅ Age 70-79 years: 1 point.

All non smokers: 0 points.

HDL cholesterol, mg/dL: 60 or higher: Minus 1 point. 50-59: 0 points. 40-49: 1 point. Under 40: 2 points.

Systolic blood pressure, mm Hg: Untreated: Under 120: 0 points. 120-129: 0 points. 130-139: 1 point. 140-159: 1 point. 160 or higher: 2 points. ⋅ Treated: Under 120: 0 points. 120-129: 1 point. 130-139: 2 points. 140-159: 2 points. 160 or higher: 3 points.

10-year risk in %: Points total: 0 point: <1%. 1-4 points: 1%. 5-6 points: 2%. 7 points: 3%. 8 points: 4%. 9 points: 5%. 10 points: 6%. 11 points: 8%. 12 points: 10%. 13 points: 12%. 14 points: 16%. 15 points: 20%. 16 points: 25%. 17 points or more: Over 30%.

In an embodiment, PCE (Goff et al., 2014; Riveros-McKay et al., 2021) includes obtaining information from the subject on one or more or all of the following: age, gender/biological sex, ethnicity, smoking status, diabetes status, HDL cholesterol level (mmol/L), total cholesterol level (mmol/L), systolic blood pressure (mm Hg), use of antihypertensive medication.

In an embodiment, in the PCE model the linear combination (L), dependent on the patient's ethnicity and gender, of the risk variables is the sum, of the product, of each variable's value and 13 coefficient, as defined in Table 5:

Linear Combination (L)=Σ(Variable Value×β coefficient).

TABLE 5
Coefficients of the Clinical Risk Variables for PCE

| Variable | Variable | β coefficient | | | |
|---|---|---|---|---|---|
| Name* | Value | Female CAU | Female AFR | Male CAU | Male AFR |
| age | In(Age) | −29.799 | 17.1141 | 12.344 | 2.469 |
| age_age | In(Age²) | 4.884 | — | — | — |
| tc | In(TC) | 13.54 | 0.9396 | 11.853 | 0.302 |
| age_tc | In(Age) × In(TC) | −3.114 | — | −2.664 | — |
| hdlc | In(HDLC) | −13.578 | −18.9196 | −7.99 | −0.307 |
| age_hdlc | In(Age) × In(HDLC) | 3.149 | 4.4748 | 1.769 | — |
| tbsp | In(TBSP) | 2.019 | 29.2907 | 1.797 | 1.916 |
| age_tsbp | In(Age) × In(TSBP) | — | −6.4321 | — | — |
| ubsp | In(UBSP) | 1.957 | 27.8197 | 1.764 | 1.809 |
| age_usbp | In(Age) × In(USBP) | — | −6.0873 | — | — |
| smoker | Smoker (0 or 1) | 7.574 | 0.6908 | 7.837 | 0.549 |
| age_smoker | In(Age) × Smoker | −1.665 | — | −1.795 | — |
| | (0 or 1) | | | | |
| diabetes | Diabetes (0 or 1) | 0.661 | 0.8738 | 0.658 | 0.645 |

*TC = total cholesterol, HDLC = high density lipoprotein cholesterol, TBSP = total systolic blood pressure, USBP = antihypertensive treatment status.

The Reynolds risk calculator takes into account a family history of premature heart disease (which implies a genetic predisposition to cardiovascular disease), and also c-reactive protein (CRP) levels (a marker of inflammation) (Ridker et al., 2007 and 2008). These two risk factors are thought to be more predictive of heart disease in women than in men. The Reynolds score is based on the following risk factors: age, current smoker, systolic blood pressure, HDL cholesterol, CRP level, mother or father with heart attack before age 60.

QRISK (the latest version being QRISK3) is a prediction algorithm for cardiovascular disease that uses traditional risk factors (age, systolic blood pressure, smoking status and ratio of total serum cholesterol to high-density lipoprotein cholesterol) together with body mass index, ethnicity, measures of deprivation, family history, chronic kidney disease, rheumatoid arthritis, atrial fibrillation, diabetes mellitus, and antihypertensive treatment (Hippisley-Cox et al., 2008). A QRISK over 10 (10% risk of CVD event over the next ten years) indicates that primary prevention with lipid lowering therapy (such as statins) should be considered.

CANRISK is a questionnaire that helps identify their risk of pre-diabetes or type 2 diabetes (Robinson et al., 2011). It is mainly for adults between the ages of 45 and 74 years, but may also be used for younger groups in high-risk populations. CANRISK includes assessment of age, gender, weight, height, body mass index, waist circumference, level of physical activity, diet, blood pressure, blood sugar levels, birth weight of children, family history of diabetes, ethnicity of parents and level of education.

### Combined Clinical Assessment×Genetic Risk

In combining the clinical risk assessment with the genetic risk assessment to obtain the “risk” of a human subject for developing coronary artery disease, atrial fibrillation or Type 2 diabetes, a maxCT approach can be used. In the embodiment, the fitted logistic regression models for CAD, atrial fibrillation and Type 2 diabetes can be:

log it(CAD)=−2.5396+3.5882×Framingham score+0.2771×PRS,

log it(AF)=−2.0551+3.3736×Framingham score+0.2074×PRS,

log it(T2D)=−2.7209+8.6933×Framingham score+0.4120×PRS.

PRS is defined as the sum of risk allele counts (0, 1, 2) of the selected SNPs weighted by their GWAS effect sizes, for example, PRS for individual i is given by:

PRSᵢ=β₁xᵢ₁+β₂xᵢ₂+ +βⱼxᵢⱼ+ . . . +βₚxᵢₚ,

where xᵢⱼ∈{0, 1, 2} are risk allele counts, βⱼ is the effect size for SNP j, and p is the total number of SNPs selected by the maxCT approach. The effect sizes of SNPs are estimated from the reported summary statistics (i.e., regression coefficients or log odds ratios with the information of rs ID, risk allele and p-value) from external published GWAS.

Similarly, using the SCT approach, the fitted logistic regression models for CAD, atrial fibrillation and Type 2 diabetes can be:

log it(CAD)=−2.6492+3.6002×Framingham score+PRS,

log it(AF)=−3.4577+3.4253×Framingham score+PRS,

log it(T2D)=−2.9857+8.6158×Framingham score+PRS,

PRS is again defined as the linear combination of the risk alleles across the selected SNPs. However, for the SCT approach, the effect sizes of the selected SNPs are not simply estimated by the reported log odd ratios but rather a linear combination of them. The linear combination is guided by testing a grid of different hyperparameters and selecting the best one that gives the best prediction performance in the training data.

The odds and the risk of diseases for a given Framingham score and PRS can also be estimated from the logistic regression model, if so desired, which are given by:

[symbol]

=exp(log it),

[symbol]

=[1+exp(−log it)]⁻¹.

In an alternate embodiment, the following formula can be used:

[Risk (i.e. Clinical Evaluation×SNP risk)]=[Clinical Evaluation risk]×SNP₁×SNP₂×SNP₃×SNP₄×SNP₅×SNP₆×SNP₇,×SNP₈, . . . ×SNP_{N} etc.

Where Clinical Evaluation is the risk provided by the clinical evaluation, and SNP₁ to SNP_{N} are the relative risk for the individual SNPs, each scaled to have a population average of 1 as outlined above. Because the SNP risk values have been “centred” to have a population average risk of 1, if one assumes independence among the SNPs, then the population average risk across all genotypes for the combined value is consistent with the underlying Clinical Evaluation risk estimate.

In an embodiment the risk of a human subject for developing coronary artery disease, atrial fibrillation or Type 2 diabetes is calculated by [Clinical Evaluation risk]×SNP₁×SNP₂×SNP₃×SNP₄×SNP₅×SNP₆×SNP₇,×SNP₈, . . . ×SNP_{N} etc. In another embodiment the risk of a human subject for developing coronary artery disease, atrial fibrillation or Type 2 diabetes is calculated by [Clinical Evaluation 5-year risk]×SNP₁×SNP₂×SNP₃×SNP₄×SNP₅×SNP₆×SNP₇,×SNP₈, . . . ×SNP_{N} etc.

In another embodiment the risk of a human subject for developing coronary artery disease, atrial fibrillation or Type 2 diabetes is calculated by [Clinical Evaluation lifetime risk]×SNP₁×SNP₂×SNP₃×SNP₄×SNP₅×SNP₆×SNP₇,×SNP₈, . . . ×SNP_{N} etc. In an embodiment, the Clinical Evaluation is performed by assessing one or more of the following: age, gender, HDL-cholesterol level (mmol/L), LDL-cholesterol level (mmol/L), total cholesterol level, blood pressure (systolic and/or diastolic (mm Hg)), smoking status, have or has had diabetes, on hypertension medication, c-reactive protein levels, whether the subject's mother or father have had a heart attack (such by the age of body mass index, ethnicity, measures of deprivation, family history, have or has had chronic kidney disease, and have or has had rheumatoid arthritis to provide a clinical risk. In another embodiment, the clinical risk assessment procedure includes obtaining information from the subject on one or more of the following: age, gender, HDL-cholesterol level (mmol/L), LDL-cholesterol level (mmol/L), total cholesterol level, blood pressure (systolic and diastolic (mm Hg)), smoking status, have or has had diabetes and on hypertension medication to provide a clinical risk. In this embodiment, the risk (i.e. combined genetic risk x clinical risk) is provided by:

[Risk (i.e. clinical×genetic risk)]=[clinical factor₁×clinical factor₂, . . . ,×clinical factor₅]×SNP₁×SNP₂×SNP₃×SNP₄×SNP₅×SNP₆×SNP₇,×SNP₈, . . . ×SNP_{N} etc.

In various embodiments the method performance is characterized by an area under the curve (AUC) of at least about 0.6, at least about 0.61, at least about 0.62, at least about 0.63, about 0.6, about 0.61, about 0.62, about 0.63, about 0.72, about 0.73, about 0.74, about 0.75, between 0.6 and 0.8, or between 0.6 and 0.76, for the risk of developing coronary artery disease.

In various embodiments the method performance is characterized by an area under the curve (AUC) of at least about 0.6, at least about 0.61, at least about 0.62, at least about 0.63, about 0.6, about 0.61, about 0.62, about 0.63, about 0.71, about 0.72, about 0.73, about 0.74, between 0.6 and 0.8, or between 0.6 and 0.75 for the risk of developing atrial fibrillation.

In various embodiments the method performance is characterized by an area under the curve (AUC) of at least about 0.77, at least about 0.78, about 0.77, about 0.78, for the risk of developing Type 2 diabetes.

In an embodiment, three calculations are required to generate the PCE probabilities (p_{ca}) defined below. These calculations use the Linear Combination (L) as defined in above and variables defined in Table 5 that are dependent on the patient's ethnicity and gender.

$$\begin{matrix}PCE probabilities & p = 1 - {S}^{exp ( L - M )} \\ Calibrated odds & A = exp ( {α}_{0} ) {( \frac{p}{1 - p} )}^{{α}_{1}} \\ Calibrated PCE probabilities & {p}_{ca} = \frac{A}{1 + A}\end{matrix}$$

The patient's result is their 10-year risk which is detailed below using the patient's PRS score, PCE probabilities, along with the variables defined in Table 6 that are dependent on the patient's ethnicity and gender.

$$10 - year Risk Score = \frac{exp ( β \times PRS ) \times {p}_{ca}}{1 - {p}_{ca} + exp ( β \times PRS ) \times {p}_{ca}}$$

TABLE 6
Final Risk Calculation Variables

| Gender | Ethnicity | S | β | M | α₀ | α₁ |
|---|---|---|---|---|---|---|
| Female | White | 0.96652 | 0.1033 | −29.1817 | 1.7257 | 1.3066 |
| Female | African | 0.95334 | 0.1033 | 86.6081 | 1.7257 | 1.3066 |
| Male | White | 0.91436 | 0.1417 | 61.1816 | 1.2439 | 1.2052 |
| Male | African | 0.89536 | 0.1417 | 19.5425 | 1.2439 | 1.2052 |

In an embodiment, when the disease is atrial fibrillation a calibrated Framingham score is determined as follows:

$$\begin{matrix}Framingham probabilities & p \\ Calibrated Framingham odds & A = exp ( {α}_{0} ) {( \frac{p}{1 - p} )}^{{α}_{1}} \\ Calibrated Framingham probabilities & {p}_{ca} = \frac{A}{1 + A}\end{matrix}$$

TABLE 7
Final Risk Calculation Variables

| Gender | β | α₀ | α₁ |
|---|---|---|---|
| Male | 0.2098 | 2.5554 | 1.6145 |
| Female | 0.1428 | 2.5352 | 1.3383 |

The patient's result is their 10-year risk which is detailed below using the patient's PRS score, Framingham probabilities, along with the variables defined in Table 7 that are dependent on the patient's ethnicity and gender.

$$10 - year Risk Score = \frac{exp ( β \times PRS ) \times {p}_{ca}}{1 - {p}_{ca} + exp ( β \times PRS ) \times {p}_{ca}}$$

In an embodiment, one or more threshold value(s) are set for determining a particular action such as the need for routine diagnostic testing or preventative therapy. For example, a score determined using a method of the invention is compared to a pre-determined threshold, and if the score is higher than the threshold a recommendation is made to take the pre-determined action. Methods of setting such thresholds have now become widely used in the art and are described in, for example, US 20140018258.

In one embodiment relating to assessing the risk of a human subject developing coronary artery disease, the subject is considered low at low risk of the 10-year Risk Score is below 7.5%, in particular below 5%, intermediate risk if the 10-year Risk Score is between 7.5% and 20%, and high if above 20%. If the subject's 10-year Risk Score is above 7.5%, in particular if above 20%, it is recommended they be given medication to treat and/or prevent hypertension.

### Subjects

The term “subject” as used herein refers to a human subject. Terms such as “subject”, “patient” or “individual” are terms that can, in context, be used interchangeably in the present disclosure. In an example, the methods of the present disclosure can be used for routine screening of subjects. Routine screening can include testing subjects at pre-determined time intervals. Exemplary time intervals include screening monthly, quarterly, six monthly, yearly, every two years or every three years.

In an embodiment, the subject has at least one symptom of coronary artery disease, atrial fibrillation or Type 2 diabetes. In another embodiment, the subject has a family history of coronary artery disease, atrial fibrillation or Type 2 diabetes.

The methods of the present disclosure can be used to assess risk in male and female subjects.

The methods of the present disclosure can be used for assessing the risk for developing coronary artery disease, atrial fibrillation or Type 2 diabetes in human subjects from various ethnic backgrounds. It is well known that over time there has been blending of different ethnic origins. While in practice, this does not influence the ability of a skilled person to practice the methods described herein, it may be desirable to identify the subject's ethnic background. In this instance, the ethnicity of the human subject can be self-reported by the subject. As an example, subjects can be asked to identify their ethnicity in response to this question: “To what ethnic group do you belong?” In another example, the ethnicity of the subject can be derived from medical records after obtaining the appropriate consent from the subject or from the opinion or observations of a clinician.

In an example, the subject can be classified as Caucasoid, Australoid, Mongoloid and Negroid based on physical anthropology. In an embodiment, the subject can be Caucasian, African American, Hispanic, Asian, Indian, or Latino. In an example, the subject is Caucasian. For example, the subject can be European.

A subject of predominantly European origin, either direct or indirect through ancestry, with white skin is considered Caucasian in the context of the present disclosure. A Caucasian may have, for example, at least 75% Caucasian ancestry (for example, but not limited to, the subject having at least three Caucasian grandparents).

A subject of predominantly central or southern African origin, either direct or indirect through ancestry, is considered Negroid in the context of the present disclosure. A Negroid may have, for example, at least 75% Negroid ancestry. An American subject with predominantly Negroid ancestry and black skin is considered African American in the context of the present disclosure. An African American may have, for example, at least 75% Negroid ancestry. Similar principle applies to, for example, subjects of Negroid ancestry living in other countries (for example Great Britain, Canada or the Netherlands).

A subject predominantly originating from Spain or a Spanish-speaking country, such as a country of Central or Southern America, either direct or indirect through ancestry, is considered Hispanic in the context of the present disclosure. A Hispanic subject may have, for example, at least 75% Hispanic ancestry.

In an embodiment, when the PCE is used the subject is self assessed as Caucasian (white), or African.

### Sample Preparation and Analysis

In performing the methods of the present disclosure, a biological sample from a subject is required. It is considered that terms such as “sample” and “specimen” are terms that can, in context, be used interchangeably in the present disclosure. Any biological material can be used as the above-mentioned sample so long as it can be derived from the subject and DNA can be isolated and analyzed according to the methods of the present disclosure. Samples are typically taken, following informed consent, from a patient by standard medical laboratory methods. The sample may be in a form taken directly from the patient, or may be at least partially processed (purified) to remove at least some non-nucleic acid material.

Exemplary “biological samples” include bodily fluids (blood, saliva, urine etc.), biopsy, tissue, and/or waste from the patient. Thus, tissue biopsies, stool, sputum, saliva, blood, lymph, tears, sweat, urine, vaginal secretions, or the like can easily be screened for SNPs, as can essentially any tissue of interest that contains the appropriate nucleic acids. In one embodiment, the biological sample is a cheek cell sample.

In another embodiment the sample is a blood sample. A blood sample can be treated to remove particular cells using various methods such as such centrifugation, affinity chromatography (e.g. immunoabsorbent means), immunoselection and filtration if required. Thus, in an example, the sample can comprise a specific cell type or mixture of cell types isolated directly from the subject or purified from a sample obtained from the subject. In an example, the biological sample is peripheral blood mononuclear cells (pBMC). Various methods of purifying sub-populations of cells are known in the art. For example, pBMC can be purified from whole blood using various known Ficoll based centrifugation methods (e.g. Ficoll-Hypaque density gradient centrifugation).

DNA can be extracted from the sample for detecting SNPs. In an example, the DNA is genomic DNA. Various methods of isolating DNA, in particular genomic DNA are known to those of skill in the art. In general, known methods involve disruption and lysis of the starting material followed by the removal of proteins and other contaminants and finally recovery of the DNA. For example, techniques involving alcohol precipitation; organic phenol/chloroform extraction and salting out have been used for many years to extract and isolate DNA. There are various commercially available kits for genomic DNA extraction (Qiagen, Life technologies; Sigma). Purity and concentration of DNA can be assessed by various methods, for example, spectrophotometry.

### Marker Detection Strategies

Amplification primers for amplifying markers (e.g., marker loci) and suitable probes to detect such markers or to genotype a sample with respect to multiple marker alleles can be used in the disclosure. For example, primer selection for long-range PCR is described in U.S. Ser. No. 10/042,406 and U.S. Ser. No. 10/236,480; for short-range PCR, U.S. Ser. No. 10/341,832 provides guidance with respect to primer selection. Also, there are publicly available programs such as “Oligo” available for primer design. With such available primer selection and design software, the publicly available human genome sequence and the polymorphism locations, one of skill in the art can construct primers to amplify the SNPs to practice the disclosure. Further, it will be appreciated that the precise probe to be used for detection of a nucleic acid comprising a SNP (e.g., an amplicon comprising the SNP) can vary, e.g., any probe that can identify the region of a marker amplicon to be detected can be used in conjunction with the present disclosure. Further, the configuration of the detection probes can, of course, vary.

As the skilled person will appreciate, the sequence of the genomic region to which these oligonucleotides hybridize can be used to design primers which are longer at the 5′ and/or 3′ end, possibly shorter at the 5′ and/or 3′ (as long as the truncated version can still be used for amplification), which have one or a few nucleotide differences (but nonetheless can still be used for amplification), or which share no sequence similarity with those provided but which are designed based on genomic sequences close to where the specifically provided oligonucleotides hybridize and which can still be used for amplification.

In some embodiments, the primers of the disclosure are radiolabelled, or labelled by any suitable means (e.g., using a non-radioactive fluorescent tag), to allow for rapid visualization of differently sized amplicons following an amplification reaction without any additional labelling step or visualization step. In some embodiments, the primers are not labelled, and the amplicons are visualized following their size resolution, e.g., following agarose or acrylamide gel electrophoresis. In some embodiments, ethidium bromide staining of the PCR amplicons following size resolution allows visualization of the different size amplicons.

It is not intended that the primers of the disclosure be limited to generating an amplicon of any particular size. For example, the primers used to amplify the marker loci and alleles herein are not limited to amplifying the entire region of the relevant locus, or any subregion thereof. The primers can generate an amplicon of any suitable length for detection. In some embodiments, marker amplification produces an amplicon at least nucleotides in length, or alternatively, at least 50 nucleotides in length, or alternatively, at least 100 nucleotides in length, or alternatively, at least 200 nucleotides in length. Amplicons of any size can be detected using the various technologies described herein. Differences in base composition or size can be detected by conventional methods such as electrophoresis.

Indeed, it will be appreciated that amplification is not a requirement for marker detection, for example one can directly detect unamplified genomic DNA simply by performing a Southern blot on a sample of genomic DNA.

Typically, molecular markers are detected by any established method available in the art, including, without limitation, allele specific hybridization (ASH), detection of single nucleotide extension, array hybridization (optionally including ASH), or other methods for detecting single nucleotide polymorphisms, amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, and single-strand conformation polymorphisms (SSCP) detection.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) and Sambrook et al. (supra).

PCR detection using dual-labelled fluorogenic oligonucleotide probes, commonly referred to as “TaqMan™” probes, can also be performed according to the present disclosure. These probes are composed of short (e.g., 20-25 bases) oligodeoxynucleotides that are labelled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO 92/02638.

Other similar methods include e.g. fluorescence resonance energy transfer between two adjacently hybridized probes, e.g., using the “LightCycler®” format described in U.S. Pat. No. 6,174,670.

Array-based detection can be performed using commercially available arrays, e.g., from Affymetrix (Santa Clara, Calif.) or other manufacturers. Reviews regarding the operation of nucleic acid arrays include Sapolsky et al. (1999); Lockhart (1998);

Fodor (1997a); Fodor (1997b) and Chee et al. (1996). Array based detection is one preferred method for identification markers of the disclosure in samples, due to the inherently high-throughput nature of array based detection.

The nucleic acid sample to be analyzed is isolated, amplified and, typically, labelled with biotin and/or a fluorescent reporter group. The labelled nucleic acid sample is then incubated with the array using a fluidics station and hybridization oven. The array can be washed and or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labelled nucleic acid, which is now bound to the probe array. Probes that most clearly match the labelled nucleic acid produce stronger signals than those that have mismatches. Since the sequence and position of each probe on the array are known, by complementarity, the identity of the nucleic acid sample applied to the probe array can be identified.

### Correlating Markers to Disease Risk

Correlations between SNPs and risk of coronary artery disease, atrial fibrillation or Type 2 diabetes can be performed by any method that can identify a relationship between an allele and increased coronary artery disease, atrial fibrillation or Type 2 diabetes risk, or a combination of alleles and increased coronary artery disease, atrial fibrillation or Type 2 diabetes risk. For example, alleles in genes or loci defined herein can be correlated with increased risk of coronary artery disease, atrial fibrillation or Type 2 diabetes. Most typically, these methods involve referencing a look up table that comprises correlations between alleles of the polymorphism and the coronary artery disease, atrial fibrillation or Type 2 diabetes risk. The table can include data for multiple allele-risk relationships and can take account of additive or other higher order effects of multiple allele-risk relationships, e.g., through the use of statistical tools such as principle component analysis, heuristic algorithms, etc.

Correlation of a marker to a coronary artery disease, atrial fibrillation or Type 2 diabetes risk optionally includes performing one or more statistical tests for correlation. Many statistical tests are known, and most are computer-implemented for ease of analysis. A variety of statistical methods of determining associations/correlations between phenotypic traits and biological markers are known and can be applied to the present disclosure. Hartl (1981). A variety of appropriate statistical models are described in Lynch and Walsh (1998). These models can, for example, provide for correlations between genotypic and phenotypic values, characterize the influence of a locus on coronary artery disease, atrial fibrillation or Type 2 diabetes risk, sort out the relationship between environment and genotype, determine dominance or penetrance of genes, determine maternal and other epigenetic effects, determine principle components in an analysis (via principle component analysis, or “PCA”), and the like. The references cited in these texts provide considerable further detail on statistical models for correlating markers and coronary artery disease, atrial fibrillation or Type 2 diabetes risk.

In addition to standard statistical methods for determining correlation, other methods that determine correlations by pattern recognition and training, such as the use of genetic algorithms, can be used to determine correlations between markers and coronary artery disease, atrial fibrillation or Type 2 diabetes risk. This is particularly useful when identifying higher order correlations between multiple alleles and coronary artery disease, atrial fibrillation or Type 2 diabetes risk. To illustrate, neural network approaches can be coupled to genetic algorithm-type programming for heuristic development of a structure-function data space model that determines correlations between genetic information and phenotypic outcomes.

In any case, essentially any statistical test can be applied in a computer implemented model, by standard programming methods, or using any of a variety of “off the shelf” software packages that perform such statistical analyses, including, for example, those noted above and those that are commercially available, e.g., from Partek Incorporated (St. Peters, Mo.; www.partek.com), e.g., that provide software for pattern recognition (e.g., which provide Partek Pro 2000 Pattern Recognition Software).

Additional details regarding association studies can be found in U.S. Ser. No. 10/106,097, U.S. Ser. No. 10/042,819, U.S. Ser. No. 10/286,417, U.S. Ser. No. 10/768,788, U.S. Ser. No. 10/447,685, U.S. Ser. No. 10/970,761, and U.S. Pat. No. 7,127,355.

Systems for performing the above correlations are also a feature of the disclosure. Typically, the system will include system instructions that correlate the presence or absence of an allele (whether detected directly or, e.g., through expression levels) with a predicted coronary artery disease, atrial fibrillation or Type 2 diabetes risk.

Optionally, the system instructions can also include software that accepts diagnostic information associated with any detected allele information, e.g., a diagnosis that a subject with the relevant allele has a particular coronary artery disease, atrial fibrillation or Type 2 diabetes risk. This software can be heuristic in nature, using such inputted associations to improve the accuracy of the look up tables and/or interpretation of the look up tables by the system. A variety of such approaches, including neural networks, Markov modelling and other statistical analysis are described above.

### Polymorphic Profiling

The disclosure provides methods of determining the polymorphic profile of an individual at the SNPs outlined in the present disclosure (Tables 1 to 4) or SNPs in linkage disequilibrium with one or more thereof.

The polymorphic profile constitutes the polymorphic forms occupying the various polymorphic sites in an individual. In a diploid genome, two polymorphic forms, the same or different from each other, usually occupy each polymorphic site. Thus, the polymorphic profile at sites X and Y can be represented in the form X (x1, x1), and Y (y1, y2), wherein x1, x1 represents two copies of allele x1 occupying site X and y1, y2 represent heterozygous alleles occupying site Y.

The polymorphic profile of an individual can be scored by comparison with the polymorphic forms associated with susceptibility to coronary artery disease, atrial fibrillation or Type 2 diabetes occurring at each site. The comparison can be performed on at least, e.g., 1, 2, 5, 10, 25, 50, or all of the polymorphic sites, and optionally, others in linkage disequilibrium with them. The polymorphic sites can be analyzed in combination with other polymorphic sites.

Polymorphic profiling is useful, for example, in selecting agents to affect treatment or prophylaxis of coronary artery disease, atrial fibrillation or Type 2 diabetes in a given individual. Individuals having similar polymorphic profiles are likely to respond to agents in a similar way.

### Computer Implemented Method

The methods of the present disclosure may be implemented by a system as a computer implemented method. For example, the system may be a computer system comprising one or a plurality of processors which may operate together (referred to for convenience as “processor”) connected to a memory. The memory may be a non-transitory computer readable medium, such as a hard drive, a solid state disk or CD-ROM. Software, that is executable instructions or program code, such as program code grouped into code modules, may be stored on the memory, and may, when executed by the processor, cause the computer system to perform functions such as determining that a task is to be performed to assist a user to determine the risk of a human subject for developing coronary artery disease, atrial fibrillation or Type 2 diabetes receiving data indicating the genetic risk and optionally the clinical risk of the subject developing coronary artery disease, atrial fibrillation or Type 2 diabetes; processing the data to obtain the risk of a human subject for developing coronary artery disease, atrial fibrillation or Type 2 diabetes; outputting the presence of the risk of a human subject for developing coronary artery disease, atrial fibrillation or Type 2 diabetes.

For example, the memory may comprise program code which when executed by the processor causes the system to determine the presence of the polymorphism(s) or receive data indicating the presence of the polymorphism(s); process the data to obtain the risk of a human subject for developing coronary artery disease, atrial fibrillation or Type 2 diabetes; report the risk of a human subject for developing coronary artery disease, atrial fibrillation or Type 2 diabetes. Thus, in an embodiment, the program code causes the system to determine the “genetic risk”.

In another example, the memory may comprise program code which when executed by the processor causes the system to determine the presence the polymorphism(s), or receive data indicating the presence polymorphism(s) and, receive or determine clinical risk data for the subject; process the data to combine the genetic risk data with the clinical risk data to obtain the risk of the subject for developing coronary artery disease, atrial fibrillation or Type 2 diabetes; report the risk of a human subject for developing coronary artery disease, atrial fibrillation or Type 2 diabetes. For example, the program code can cause the system to combine clinical risk assessment data×genetic risk.

In another embodiment, the system may be coupled to a user interface to enable the system to receive information from a user and/or to output or display information.

For example, the user interface may comprise a graphical user interface, a voice user interface or a touchscreen. In an example, the user interface is a SNP array platform.

In an embodiment, the system may be configured to communicate with at least one remote device or server across a communications network such as a wireless communications network. For example, the system may be configured to receive information from the device or server across the communications network and to transmit information to the same or a different device or server across the communications network. In other embodiments, the system may be isolated from direct user interaction.

In another embodiment, performing the methods of the present disclosure to assess the risk of a subject for developing coronary artery disease, atrial fibrillation or

Type 2 diabetes, enables establishment of a diagnostic or prognostic rule based on the the genetic risk of the subject developing coronary artery disease, atrial fibrillation or Type 2 diabetes. For example, the diagnostic or prognostic rule can be based on the genetic risk relative to a control, standard or threshold level of risk. In another example, the diagnostic or prognostic rule can be based on the combined genetic and clinical risk relative to a control, standard or threshold level of risk.

In another embodiment, the diagnostic or prognostic rule is based on the application of a statistical and machine learning algorithm. Such an algorithm uses relationships between a population of SNPs and disease status observed in training data (with known disease status) to infer relationships which are then used to determine the risk of a human subject for developing coronary artery disease, atrial fibrillation or Type 2 diabetes in subjects with an unknown risk. An algorithm is employed which provides a risk of a human subject developing coronary artery disease, atrial fibrillation or Type 2 diabetes. The algorithm performs a multivariate or univariate analysis function.

### Kits and Products

In an embodiment, the present disclosure provides a kit comprising at least one set of primers for amplifying two or more nucleic acids, wherein the two or more nucleic acids comprise a polymorphism selected from any one of Tables 1 to 4, or any combinations thereof such as Tables, 1, 3 and 4, or Tables 1 and 2, or a single nucleotide polymorphism in linkage disequilibrium with one or more thereof.

In an embodiment, the kit is for detecting, in a biological sample derived from a human subject, the presence of at least one polymorphisms associated with a risk of developing coronary artery disease, wherein the at least one polymorphism is selected from Table 1 and/or Table 2, or a single nucleotide polymorphism in linkage disequilibrium with one or more thereof. In an embodiment, the kit comprises sets of primers for detecting at least three, at least four, at least five, at least six, at least seven, at least eight, at least nine, at least ten, at least 20, at least 50, at least 100, at least 150, at least 200, at least 250, at least 300, at least 350, at least 400, at least 450, at least 500, at least 550, at least 600, at least 650, at least 700, at least 750, at least 800, at least 825, at least 850, at least 900, at least 950, at least 1,000, or all of the polymorphism provided in Table 1 and/or Table 2, or a single nucleotide polymorphism in linkage disequilibrium with one or more thereof. In an embodiment, the kit comprises sets of primers for detecting each of the polymorphisms provided in Table 1 and/or Table 2, or all of the polymorphisms in Tables 1 and 2.

In an embodiment, the kit is for detecting, in a biological sample derived from a human subject, the presence at least one polymorphisms associated with a risk of developing atrial fibrillation, wherein the at least one polymorphism is selected from Table 3, or a single nucleotide polymorphism in linkage disequilibrium with one or more thereof. In an embodiment, the kit comprises sets of primers for detecting at least two, at least three, at least four, at least five, at least six, at least seven, at least eight, at least nine, at least ten, at least 20, at least 50, at least 100, at least 125, at least 150, at least 175, at least 200, at least 225, at least 250, or all of the polymorphism provided in Table 3, or a single nucleotide polymorphism in linkage disequilibrium with one or more thereof. In an embodiment, the kit comprises sets of primers for detecting each of the polymorphism provided in Table 3.

In another embodiment, the kit is for detecting, in a biological sample derived from a human subject, the presence at least one polymorphisms associated with a risk of developing Type 2 diabetes, wherein the at least one polymorphism is selected from Table 4, or a single nucleotide polymorphism in linkage disequilibrium with one or more thereof. In an embodiment, the kit comprises sets of primers for detecting at least three, at least four, at least five, at least six, at least seven, at least eight, at least nine, at least ten, at least 20, at least 30, at least 40, at least 50, at least 60, at least 70, at least 80, at least 85, or all of the polymorphism provided in Table 4, or a single nucleotide polymorphism in linkage disequilibrium with one or more thereof. In an embodiment, the kit comprises sets of primers for detecting each of the polymorphism provided in Table 4.

As would be appreciated by those of skill in the art, once a SNP is identified, primers can be designed to amplify the SNP as a matter of routine. Various software programs are freely available that can suggest suitable primers for amplifying SNPs of interest.

Again, it would be known to those of skill in the art that PCR primers of a PCR primer pair can be designed to specifically amplify a region of interest from human DNA. In the context of the present disclosure, the region of interest contains the single-base variation (e.g. single-nucleotide polymorphism, SNP) which shall be genotyped. Each PCR primer of a PCR primer pair can be placed adjacent to a particular single-base variation on opposing sites of the DNA sequence variation. Furthermore, PCR primers can be designed to avoid any known DNA sequence variation and repetitive DNA sequences in their PCR primer binding sites.

The kit may further comprise other reagents required to perform an amplification reaction such as a buffer, nucleotides and/or a polymerase, as well as reagents for extracting nucleic acids from a sample.

Array based detection is one preferred method for assessing the SNPs of the disclosure in samples, due to the inherently high-throughput nature of array based detection. A variety of probe arrays have been described in the literature and can be used in the context of the present disclosure for detection of SNPs that can be correlated to coronary artery disease, atrial fibrillation or Type 2 diabetes. For example, DNA probe array chips are used in one embodiment of the disclosure. The recognition of sample DNA by the set of DNA probes takes place through DNA hybridization. When a DNA sample hybridizes with an array of DNA probes, the sample binds to those probes that are complementary to the sample DNA sequence. By evaluating to which probes the sample DNA for an individual hybridizes more strongly, it is possible to determine whether a known sequence of nucleic acid is present or not in the sample, thereby determining whether a marker found in the nucleic acid is present.

Thus, in another embodiment, the present disclosure provides a genetic array comprising at least two sets of probes for hybridising to two or more nucleic acids, wherein the two or more nucleic acids comprise a polymorphism selected from any one of Tables 1 to 4, or any combinations thereof such as Tables, 1, 3 and 4, or Tables 1 and 2, or a single nucleotide polymorphism in linkage disequilibrium with one or more thereof.

In an embodiment, the genetic array is for detecting, in a biological sample derived from a human subject, the presence of at least one polymorphisms associated with a risk of developing coronary artery disease, wherein the at least one polymorphism is selected from Table 1 and/or Table 2, or a single nucleotide polymorphism in linkage disequilibrium with one or more thereof. In an embodiment, the genetic array comprises probes for detecting at least three, at least four, at least five, at least six, at least seven, at least eight, at least nine, at least ten, at least 20, at least 50, at least 100, at least 150, at least 200, at least 250, at least 300, at least 350, at least 400, at least 450, at least 500, at least 550, at least 600, at least 650, at least 700, at least 750, at least 800, at least 825, at least 850, at least 900, at least 950, at least 1,000, or all of the polymorphism provided in Table 1 and/or Table 2, or a single nucleotide polymorphism in linkage disequilibrium with one or more thereof. In an embodiment, the genetic array comprises probes for detecting each of the polymorphisms provided in Table 1 and/or Table 2, or all of the polymorphisms in Tables 1 and 2.

In an embodiment, the genetic array is for detecting, in a biological sample derived from a human subject, the presence at least one polymorphisms associated with a risk of developing atrial fibrillation, wherein the at least one polymorphism is selected from Table 3, or a single nucleotide polymorphism in linkage disequilibrium with one or more thereof. In an embodiment, the genetic array comprises probes for detecting at least two, at least three, at least four, at least five, at least six, at least seven, at least eight, at least nine, at least ten, at least 20, at least 50, at least 100, at least 125, at least 150, at least 175, at least 200, at least 225, at least 250, or all of the polymorphism provided in Table 3, or a single nucleotide polymorphism in linkage disequilibrium with one or more thereof. In an embodiment, the genetic array comprises probes for detecting each of the polymorphism provided in Table 3.

In another embodiment, the genetic array is for detecting, in a biological sample derived from a human subject, the presence at least one polymorphisms associated with a risk of developing Type 2 diabetes, wherein the at least one polymorphism is selected from Table 4, or a single nucleotide polymorphism in linkage disequilibrium with one or more thereof. In an embodiment, the genetic array comprises probes for detecting at least three, at least four, at least five, at least six, at least seven, at least eight, at least nine, at least ten, at least 20, at least 30, at least 40, at least 50, at least 60, at least 70, at least 80, at least 85, or all of the polymorphism provided in Table 4, or a single nucleotide polymorphism in linkage disequilibrium with one or more thereof. In an embodiment, the genetic array comprises probes for detecting each of the polymorphism provided in Table 4.

In an embodiment, the array comprises less than 100,000, less than 50,000, less than 25,000, less than 10,000, less than 5,000, less than 2,500 or less than 1,000 specific nucleic acids.

Primers and probes for other SNPs can be included with the above exemplified kits/arrays.

### EXAMPLES

### Example 1—Materials and Methods

The inventors used data from the UK Biobank Axiom Array (Bycroft et al., 2018) to develop polygenic risk scores (PRSs) for five common diseases (Said et al., 2018; Eastwood et al., 2016): CAD, hypertension, atrial fibrillation, stroke and type 2 diabetes. The UK Biobank conducted baseline assessment of over 500,000 participants aged 40-69 years from 2006 to 2010. For quality control, the inventors removed variants with minor allele frequency less than 0.001, Hardy-Weinberg equilibrium p-value less than 10⁻⁵, and genotyping rate at least 95%. After quality control and exclusion of prevalent cases, the data we used for analysis consisted of 315,327 individuals and 602,976 SNPs.

To adjust for the effect of age and prevent extreme unbalanced case-control ratio, the inventors applied the following resampling strategy to create training and testing sets for our study. For each of the diseases, the inventors computed the quintiles of age and divided individuals into five age groups. For each of the five age groups, and for each gender separately, we drew (at most) 5 controls for each case (i.e., if the number of controls was not enough to draw 5 controls per case for all age groups, the inventors drew 4 per case, and so on). By this resampling strategy, the individuals are age and sex matched, and the size difference between the number of cases and controls is largely reduced.

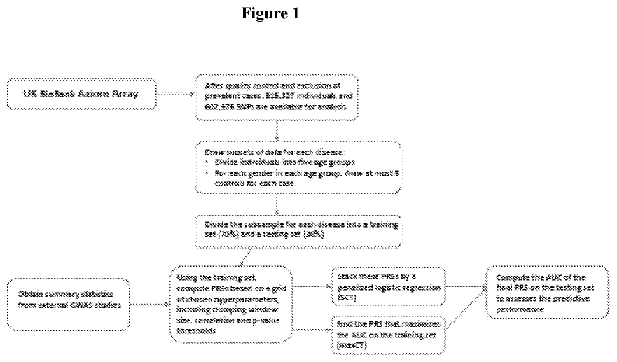

After resampling, the data was split into training (70%) and testing (30%) sets for each disease, with roughly the same case-control ratio. The sizes of the training and testing sets are summarized in Table 8. The training sets were used to build a PRS for each disease and the predictive performances were evaluated on the testing sets. The overall workflow is summarized in FIG. 1.

TABLE 8
Sizes of training and testing sets used in our study. The numbers in
parentheses are the number of controls and cases, respectively.

| Disease | Training size | Testing size |
|---|---|---|
| CAD | 38,217 (31,847/6,370) | 16,377 (13,648/2,729) |
| Hypertension | 51,273 (41,018/10,255) | 21,972 (17,578/4,394) |
| Atrial fibrillation | 28,725 (23,937/4,788) | 12,309 (10,258/2,051) |
| Stroke | 12,693 (10,577/2,116) | 5,439 (4,533/906) |
| Type 2 diabetes | 12,768 (10,640/2,128) | 5,472 (4,560/912) |

The inventors created our PRSs using a recently developed method called stacked clumping and thresholding (SCT) (Prive et al., 2019). Apply clumping (or pruning) to control linkage disequilibrium and then followed by marginal p-value thresholding is a standard method for computing PRS9. This approach requires users to specify hyperparameters such as the size of clumping windows (kb), the correlation threshold (r2) and the p-value significance threshold for clumped SNPs. In general, it is not straightforward how to choose these hyperparameters in practice. Usually, users apply some default values for these hyperparameters, for example, the default option in Plink (Purcell et al., 2007) uses r2=0:5 for the correlation threshold, 250 kb for the window size and p=0:01 for the p-value threshold.

SCT is a more general algorithm that is based on the standard clumping and thresholding method. It chooses a set of hyperparameters, runs clumping and thresholding on each combination of those parameters and gives a PRS for each combination. Table 9 shows an example of these hyperparameters values; these are the default values used in the R package bigsnpr. The PRSs are then stacked using a penalized regression model. The outcome of this algorithm is a linear combination of PRSs, where each PRS is also a linear combination of variants. Therefore, a single vector of variant effect sizes can be obtained in the final prediction model. Instead of stacking these PRSs, we could also select the PRS with the best prediction, and this is referred to the maxCT approach. In general, SCT would identify more genetic variants than maxCT.

### Example 2—Results

The inventors applied SCT and maxCT to create PRSs for five common diseases using UK Biobank data. They used training sets to create 1,400 risk scores for each chromosome using different values of hyperparameters, as listed in Table 9. For maxCT, they selected the risk score that maximized the AUC on the training sets as the final PRS. For SCT, they stacked those 30,800 (1,400×22) risk scores from all 22 chromosomes by a penalized logistic regression, the optimal stack weight was also estimated from the training sets. These steps were conducted using the R package bigsnpr.

TABLE 9
A grid of different hyperparameters used in the SCT algorithm (the default
values used in the R package bigsnpr). The algorithm runs clumping and
thresholding on each combination of these parameters and combines the
risk scores by a penalized regression. The window size is computed
as the base windows size divided by the correlation threshold.

| Hyperparameters | Values |
|---|---|
| Correlation threshold (r²) | 0.01, 0.05, 0.1, 0.2, 0.5, 0.8, 0.95 |
| Base window sizes (kb) | 50, 100, 200, 500 |
| Significance threshold (p) | 50 evenly spaced thresholds on a |
| | logarithmic scale |

To estimate the GWAS effect sizes of SNPs, the inventors obtained summary statistics from large external GWAS. Ambiguous SNPs and variants with duplicated positions or refSNP cluster ID numbers were removed, only keeping SNPs that appeared in both the UK Biobank data and the study from which the inventors used summary statistics. These GWAS and the number of SNPs are summarized in Table 10.

TABLE 10
External GWAS summary statistics used in our study, with the number
of SNPs in the original studies. The last column shows the number
of SNPs that appeared in both the UK Biobank data and the
original studies after removing ambiguous SNPs and other QC.

| Disease | GWAS study | # SNPs | # Matched SNPs |
|---|---|---|---|
| Coronary artery | Nikpay et al. (2015) | 9,455,778 | 506,432 |
| disease | | | |
| Hypertension | Zhu et al.(2019) | 5,265,189 | 382,924 |
| Atrial fibrillation | Christophersen | 11,792,062 | 508,687 |
| | et al.(2017) | | |
| Stroke | Malik et al.(2018) | 8,255,860 | 513,802 |
| Type 2 diabetes | Scott et al.(2017) | 12,056,346 | 536,788 |

For each disease, the inventors created the risk scores using three different approaches. A risk prediction model was built by (i) using only the genetic variants, (ii) using the Framingham score (D'Agostino et al., 1994 and 2008; Wilson et al., 1998 and 2007, Wolf et al., 1991; Schnabel et al., 2009; Parikh et al. 2008), which uses clinical factors for estimating risk, and (iii) using genetic variants together with the Framingham score. The aim was to examine the predictive performance of genetic variants without other covariates such as age, and to verify if we can improve risk prediction by combining genetic scores with the clinical factor. After these risk scores are created for each disease, their predictive power on the testing data was quantified by computing AUC.

The main results are summarized in Table 11. For CAD, the AUCs of the risk scores using only genetic variants were 0.58 (95% CI: [0.57-0.59]) with maxCT and 0.60 (95% CI: [0.59-0.61]) with SCT. Similar AUC values were found in the model that used the Framingham score as the only predictor, which had an AUC of 0.59 (95% CI: [0.58-0.60]). Furthermore, the clinical risk prediction is improved when the PRS was combined with the Framingham score; the AUC is increased from 0.59 to 0.62 (95% CI: [0.60-0.63]) with maxCT and 0.63 (95% CI: [0.62-0.64]) with SCT. Table 12 shows the numbers of SNPs identified by both methods and Table 13 gives the optimal hyperparameters used in maxCT. SCT identified a set of 389,606 variants that best predicted CAD, while maxCT identified 867 variants, both when the Framingham score was present in the prediction model.

TABLE 11
AUCs (95% CI) of risk prediction scores created by three approaches, which
are based on (i) the Framingham score only, (ii) the genetic variants only,
and (iii) combining genetic variants with the Framingham score.

| | Framingham | SNPs only | | Framingham score + SNPs | |
|---|---|---|---|---|---|
| Disease | score only | maxCT | SCT | maxCT | SCT |
| Coronary artery | 0.59 (0.58-0.60) | 0.58 (0.57-0.59) | 0.50 (0.50-0.61) | 0.62 (0.60-0.63) | 0.63 (0.62-0.64) |
| disease | | | | | |
| Hypertension | 0.72 (0.71-0.73) | 0.58 (0.57-0.59) | 0.59 (0.58-0.60) | 0.72 (0.72-0.73) | 0.72 (0.72-0.73) |
| Atrial fibrillation | 0.54 (0.53-0.56) | 0.60 (0.59-0.61) | 0.61 (0.60-0.63) | 0.61 (0.60-0.63) | 0.63 (0.61-0.64) |
| Stroke | 0.56 (0.54-0.59) | 0.52 (0.50-0.54) | 0.52 (0.49-0.54) | 0.56 (0.54-0.58) | 0.57 (0.54-0.59) |
| Type 2 diabetes | 0.77 (0.75-0.79) | 0.59 (0.57-0.61) | 0.60 (0.58-0.62) | 0.78 (0.76-0.79) | 0.78 (0.76-0.79) |

Strong predictive performance was also found for atrial fibrillation. Compared with the Framingham risk model, which has an AUC of 0.54 (95% CI: [0.53-0.56]), the risk scores based on genetic variants provided much better prediction, having an AUC of 0.60 (95% CI: [0.59-0.61]) with maxCT and 0.61 (95% CI: [0.60-0.63]) with SCT. The inventors also found combining the PRS with the Framingham score substantially increased the AUC from 0.54 to 0.61 (95% CI: [0.60-0.63]) with maxCT and 0.63 (95% CI: [0.61-0.64]) with SCT. In addition, the strong performance of both methods, especially maxCT, are obtained using much fewer variants compared with other studies. For example, the PRS for atrial fibrillation developed in Khera et al. (2018) has 6,730,541 SNPs while our PRS has 225,032 variants identified by SCT and 265 variants identified by maxCT.

TABLE 12
Number of SNPs identified by the SCT and maxCT
method with and without adjusting for the
Framingham score. The 867 polymorphisms analysed for
CAD are provided in Table 1, the 265 polymorphisms
analysed for atrial fibrillation are provided in Table 3, and the
90 polymorphisms analysed for T2D are provided in Table 4.

| | SNPs only | | Framingham score + SNPs | |
|---|---|---|---|---|
| Disease | maxCT | SCT | maxCT | SCT |
| CAD | 1,059 | 390,782 | 867 | 389,606 |
| Hypertension | 61,669 | 309,759 | 153,454 | 309,208 |
| Atrial fibrillation | 265 | 216,837 | 265 | 225,032 |
| Stroke | 17,568 | 169,186 | 12,518 | 122,509 |
| Type 2 diabetes | 46,353 | 419,209 | 90 | 387,010 |

TABLE 13
Optimal hyperparameters for maxCT that
maximized AUC in the training sets with
and without adjusting for the Framingham score.
These hyperparameters are the size of clumping
windows (kb), the correlation threshold (r2) and the
log p-value significance threshold for the clumped SNPs.

| | SNPs only | | | Framingham score + SNPs | | |
|---|---|---|---|---|---|---|
| Disease | r² | kb | log(p) | r² | kb | log(p) |
| CAD | 0.50 | 1,000 | 2.91 | 0.20 | 250 | 2.91 |
| Hypertension | 0.80 | 625 | 1.01 | 0.80 | 625 | 0.47 |
| Atrial fibrillation | 0.95 | 52 | 4.24 | 0.95 | 52 | 4.24 |
| Stroke | 0.95 | 105 | 1.55 | 0.95 | 526 | 1.71 |
| Type 2 diabetes | 0.80 | 125 | 1.12 | 0.20 | 500 | 5.38 |

For hypertension and type 2 diabetes, the Framingham score provided a very strong predictive performance, having AUCs of 0.72 (95% CI: [0.71-0.73]) and 0.77 (95% CI: [0.75-0.79]) respectively. For these two diseases, the clinical factor outperformed the PRSs, which had AUCs of 0.59 (95% CI: [0.58-0.60]) for hypertension and 0.60 (95% CI: [0.58-0.62]) for type 2 diabetes, both with SCT. Adding the PRS increased the AUC for type 2 diabetes from 0.77 to 0.78 (95% CI: [0.76-0.79]) and no meaningful improvement was observed for hypertension.

For stroke, the predictive performance of PRS was weak compared with the previous diseases. The risk scores generated by the three approaches all have AUC less than 0.60; in fact, the PRS only has an AUC of 0.52 (95% CI: [0.49-0.54]) with SCT. The Framingham score has an AUC of 0.56 (95% CI: [0.54-0.59]) and the best AUC among all risk scores was found to be 0.57 (95% CI: [0.54-0.59]).

To provide another way of interpreting the strength of risk prediction, we also computed the odds per adjusted standard deviation (OPERA) (Hopper et al., 2015) for the Framingham score, the PRS based on genetic variants by SCT and the combination of these two risk scores. OPERA is a measure to access the ability of a risk factor to discriminate between cases and controls on a population basis, it uses the standard deviation of the residuals for controls after adjusting for all other risk factors (such as age or sex). In this case, because age and sex are already adjusted by the design, the inventors did not put them in the prediction model when computing the OPERA. The results are summarized in Table 14.

TABLE 14
Odds per adjusted standard deviation (OPERA) for age, Framingham
score, PRS (generated by SCT using SNPs only), and the
joint risk score of the Framingham score and the PRS.

| | | Framingham | | Framingbarn |
|---|---|---|---|---|
| Disease | Age | score | PRS | score + PRS |
| CAD | 1.05 (1.01-1.10) | 1.31 (1.27-1.36) | 1.43 (1.37-1.49) | 1.56 (1.50-1.62) |
| Hypertension | 1.04 (1.00-1.07) | 2.20 (2.12-2.28) | 1.36 (1.31-1.40) | 2.27 (2.19-2.35) |
| Atrial fibrillation | 1.10 (1.05-1.16) | 1.19 (1.14-1.24) | 1.50 (1.43-1.58) | 1.57 (1.50-165) |
| Stroke | 1.13 (1.03-1.20) | 1.23 (1.15-1.31) | 1.06 (0.99-1.14) | 1.23 (1.16-1.32) |
| Type 2 diabetes | 0.99 (0.93-1.07) | 2.02 (1.90-2.14) | 1.45 (1.35-1.56) | 2.39 (2.06-2.34) |

To reassure age had been taken into account by our resampling strategy, the inventors also added the OPERA of age in the first column. The OPERA of age are close to 1 for all five common diseases, equivalently it means the risk gradients of age are close to 0:5 in terms of AUC. In contrast, without this design, the inventors found that using age only to predict atrial fibrillation has an AUC of 0.69, this shows why high AUCs can be misleading if age is simply included in the predictions.

In terms of the OPERA for the Framingham score and the PRS, the results are similar to what the inventors observed before. For example, the Framingham score has strong predictive power for hypertension and type 2 diabetes, having an OPERA of 2.20 (95% CI: [2.12-2.28]) and 2.02 (95% CI: [1.90-2.14]) respectively. The PRS has better predictive power than the Framingham score for CAD and atrial fibrillation, having an OPERA of 1.43 (95% CI: [1.37-1.49]) and 1.50 (95% CI: [1.43-1.58]) respectively, while the Framingham score has an OPERA of 1.31 (95% CI: [1.27-1.36]) for CAD and 1.19 (95% CI: [1.14-1.24]) for atrial fibrillation. As before, the inventors found improvement in predictions when combing the PRS with the Framingham score, the OPERA is increased from 1.31 (95% CI: [1.27-1.36]) to 1.56 (95% CI: [1.50-1.62]) for CAD and from 1.19 (95% CI: [1.14-1.24]) to 1.57 (95% CI: [1.50-1.65]) for atrial fibrillation. No meaningful improvement was found for stroke and hypertension.

The inventors also found combining the PRS with the PCE score (Goff et al., 2014; Riveros-McKay et al., 2021) (and as described herein) further improved test performance compared with the Framingham plus SNPs models, with an increase in AUC from 0.62 (95% CI 0.60-0.63) to 0.75 (95% CI 0.74-0.76). The PCE data included the SNPs in Table 2 in addition to those in Table 1.

Furthermore, the inventors determined that using a calibrated Framingham score (as described herein) improved the performance of the test for atrial fibrillation from an AUC of 0.7184 to 0.7361.

The present application claims priority from AU 2020903793 filed 20 Oct. 2020, the entire contents of which are incorporation herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Bycroft et al. (2018). Nature 562:203-209.
Chee et al. (1996) Science 274:610-614.
Christophersen et al. (2017) Nature Genetics 49:946-952.
Devlin and Risch (1995) Genomics. 29: 311-322.
D'Agostino et al. (1994) Stroke 25:40-43.
D'Agostino et al. (2008) Circulation 117:743-753.
Eastwood et al. (2016) PloS One 11, e0162388.
Fodor (1997a) FASEB Journal 11:A879.
Fodor (1997b) Science 277: 393-395.
Goff et al. (2014) Circulation 129:S49-S73.
Hard (1981) A Primer of Population Genetics Washington University, Saint Louis Sinauer Associates, Inc. Sunderland, Mass. ISBN: 0-087893-271-2.
Hippisley-Cox et al. (2008) Heart 94: 34-39.
Hopper (2015). American Journal of Epidemiology 182:863-867.
Khera et al. (2018) Nature Genomics 50:1219-1224.
Lockhart (1998) Nature Medicine 4:1235-1236.
Lynch and Walsh (1998) Genetics and Analysis of Quantitative Traits, Sinauer Associates, Inc. Sunderland Mass. ISBN 0-87893-481-2.
Malik et al. (2018) Nature Genetics 50:524-537.
Mavaddat et al. (2015) J Natl Cancer Inst 107:40/036.
Nikpay et al. (2015) Nature Genetics 47:1121.
Parikh et al. (2008) Annals of Internal Medicine 148:102-110.
Pencina et al. (2008) Stat Med 27:157-172.
Privé et al. (2019) The American Journal of Human Genetics 105:1213-1221.
Purcell et al. (2007) The American Journal of Human Genetics 81:559-575.
Ridker et al. (2007) JAMA. 297:611-619.
Ridker et al. (2008) Circulation 118:2243-2251.
Riveros-McKay et al. (2021) Circ Genom Precis Med 14(2):e003304. doi: 10.1161/CIRCGEN.120.003304.
Robinson et al. (2011) Chronic Dis Inj Can 32:19-31.
Said et al. (2018) JAMA Cardiology 3:693-702.
Sapolsky et al. (1999) Genet Anal: Biomolec Engin 14:187-192.
Schnabel et al. (2009) The Lancet 373:739-745.
Scott et al. (2017) Diabetes 66:2888-2902.
Slatkin and Excoffier (1996) Heredity 76: 377-383.
Tijssen (1993) Laboratory Techniques in Biochemistry and Molecular Biology—Hybridization with Nucleic Acid Probes Elsevier, New York.
Wilson et al. (1998) Circulation 97:1837-1847.
Wilson et al. (2007) Archives of Internal Medicine 167:1068-1074.
Wolf et al. (1991) Stroke 22:312-318.
Zhu et al. (2019) Respiratory Research 20:64.

1. A method for assessing the risk of a human subject developing coronary artery disease, the method comprising performing a genetic risk assessment of the human subject, wherein the genetic risk assessment involves detecting, in a biological sample derived from the human subject, the presence of at least one polymorphisms associated with a risk of developing coronary artery disease, wherein the at least one polymorphism is selected from Table 1 and/or Table 2, or a single nucleotide polymorphism in linkage disequilibrium with one or more thereof.

2. The method of claim 1 which comprises detecting the presence of at least two, at least three, at least four, at least five, at least six, at least seven, at least eight, at least nine, at least ten, at least 20, at least 50, at least 100, at least 150, at least 200, at least 250, at least 300, at least 350, at least 400, at least 450, at least 500, at least 550, at least 600, at least 650, at least 700, at least 750, at least 800, at least 825, at least 850, or all of the polymorphism provided in Table 1 and/or Table 2, or a single nucleotide polymorphism in linkage disequilibrium with one or more thereof.

3. The method of claim 1 which comprises detecting each of the polymorphisms provided in Table 1.

4. The method of claim 3 which further comprises detecting each of the polymorphisms provided in Table 2.

5. A method for assessing the risk of a human subject developing atrial fibrillation, the method comprising performing a genetic risk assessment of the human subject, wherein the genetic risk assessment involves detecting, in a biological sample derived from the human subject, the presence at least one polymorphisms associated with a risk of developing atrial fibrillation, wherein the at least one polymorphism is selected from Table 3, or a single nucleotide polymorphism in linkage disequilibrium with one or more thereof.

6. The method of claim 5 which comprises detecting the presence of at least two, at least three, at least four, at least five, at least six, at least seven, at least eight, at least nine, at least ten, at least 20, at least 50, at least 100, at least 125, at least 150, at least 175, at least 200, at least 225, at least 250, or all of the polymorphism provided in Table 3, or a single nucleotide polymorphism in linkage disequilibrium with one or more thereof.

7. The method of claim 5 which comprises detecting each of the polymorphism provided in Table 3.

8. A method for assessing the risk of a human subject developing Type 2 diabetes, the method comprising performing a genetic risk assessment of the human subject, wherein the genetic risk assessment involves detecting, in a biological sample derived from the human subject, the presence at least one polymorphisms associated with a risk of developing Type 2 diabetes, wherein the at least one polymorphism is selected from Table 4, or a single nucleotide polymorphism in linkage disequilibrium with one or more thereof.

9. The method of claim 8 which comprises detecting the presence of at least two, at least three, at least four, at least five, at least six, at least seven, at least eight, at least nine, at least ten, at least 20, at least 30, at least 40, at least 50, at least 60, at least 70, at least 80, at least 85, or all of the polymorphism provided in Table 4, or a single nucleotide polymorphism in linkage disequilibrium with one or more thereof.

10. The method of claim 8 which comprises detecting each of the polymorphism provided in Table 4.

11. The method according to any one of claims 1 to 10 which further comprises
performing a clinical risk assessment of the human subject; and
combining the clinical risk assessment and the genetic risk assessment to obtain the risk of a human subject developing coronary artery disease, atrial fibrillation or Type 2 diabetes.

12. The method of claim 11, wherein the clinical risk assessment includes obtaining information from the subject on one or more or all of: age, gender, HDL-cholesterol level (mmol/L), LDL-cholesterol level (mmol/L), total cholesterol level, blood pressure (systolic and/or diastolic (mm Hg)), smoking status, have or has had diabetes, on hypertension medication, c-reactive protein levels, whether the subject's mother or father have had a heart attack (such by the age of 60), body mass index, ethnicity, measures of deprivation, family history, have or has had chronic kidney disease, and have or has had rheumatoid arthritis.

13. The method of claim 11 or claim 12, wherein the clinical risk assessment includes obtaining information from the subject on one or more or all of: age, gender, HDL-cholesterol level (mmol/L), LDL-cholesterol level (mmol/L), total cholesterol level, blood pressure (systolic and diastolic (mm Hg)), smoking status, have or has had diabetes and on hypertension medication.

14. The method of claim 13, wherein the clinical risk assessment is the Framingham score.

15. The method according to any one of claims 1 to 4 or 11 to 14, wherein the clinical risk assessment is The American College of Cardiologists Pooled Cohort Equations (PCE).

16. The method according to any one of claims 11 to 15, wherein combining the clinical risk assessment and the genetic risk assessment comprises adding or multiplying the risk assessments.

17. A method of determining the identity of the alleles of fewer than 100,000 polymorphisms in a human subject selected from the group of subjects consisting of humans in need of assessment for the risk of developing coronary artery disease to produce a polymorphic profile of the subject, comprising
(i) selecting for allelic identity analysis at least one polymorphism provided in Table 1 and/or Table 2, or a single nucleotide polymorphism in linkage disequilibrium with one or more thereof,
(ii) detecting, in a biological sample derived from the human subject, the polymorphisms, and
(iii) producing the polymorphic profile of the subject screening based on the identity of the alleles analysed in step (ii), wherein fewer than 100,000 polymorphisms are selected for allelic identity analysis in step (i) and the same fewer than 100,000 polymorphisms are analysed in step (ii).

18. A method of determining the identity of the alleles of fewer than 100,000 polymorphisms in a human subject selected from the group of subjects consisting of humans in need of assessment for the risk of developing atrial fibrillation to produce a polymorphic profile of the subject, comprising
(i) selecting for allelic identity analysis at least one polymorphism provided in Table 3, or a single nucleotide polymorphism in linkage disequilibrium with one or more thereof,
(ii) detecting, in a biological sample derived from the human subject, the polymorphisms, and
(iii) producing the polymorphic profile of the subject screening based on the identity of the alleles analysed in step (ii), wherein fewer than 100,000 polymorphisms are selected for allelic identity analysis in step (i) and the same fewer than 100,000 polymorphisms are analysed in step (ii).

19. A method of determining the identity of the alleles of fewer than 100,000 polymorphisms in a human subject selected from the group of subjects consisting of humans in need of assessment for the risk of developing Type 2 diabetes to produce a polymorphic profile of the subject, comprising
(i) selecting for allelic identity analysis at least one polymorphism provided in Table 4, or a single nucleotide polymorphism in linkage disequilibrium with one or more thereof,
(ii) detecting, in a biological sample derived from the human subject, the polymorphisms, and
(iii) producing the polymorphic profile of the subject screening based on the identity of the alleles analysed in step (ii), wherein fewer than 100,000 polymorphisms are selected for allelic identity analysis in step (i) and the same fewer than 100,000 polymorphisms are analysed in step (ii).

20. The method according to any one of claims 1 to 19, wherein the polymorphism(s) in linkage disequilibrium has a linkage disequilibrium above 0.9.

21. The method according to any one of claims 1 to 20, wherein the polymorphism(s) in linkage disequilibrium has a linkage disequilibrium of 1.

22. The method according to any one of claims 1 to 21, wherein the risk assessment produces a score and the method further comprises comparing the score to a predetermined threshold, wherein if the score is at, or above, the threshold the subject is assessed at being at risk of developing coronary artery disease, atrial fibrillation or Type 2 diabetes.

23. A method for determining the need for routine diagnostic testing of a human subject for a coronary artery disease, atrial fibrillation or Type 2 diabetes, the method comprising assessing the risk of the subject for developing coronary artery disease, atrial fibrillation or Type 2 diabetes using the method according to any one of claims 1 to 22.

24. A method of screening for coronary artery disease, atrial fibrillation or Type 2 diabetes in a human subject, the method comprising assessing the risk of the subject for developing coronary artery disease, atrial fibrillation or Type 2 diabetes using the method according to any one of claims 1 to 22, and routinely screening for coronary artery disease, atrial fibrillation or Type 2 diabetes in the subject if they are assessed as having a risk for developing coronary artery disease, atrial fibrillation or Type 2 diabetes.

25. The method of claim 23 or claim 24, wherein for coronary artery disease the screening involves conducting one or more of an electrocardiogram (ECG), an exercise stress test, a nuclear stress test, a cardiac catheterization and angiogram, or a cardiac CT scan.

26. The method of claim 23 or claim 24, wherein for atrial fibrillation disease the screening involves conducting an electrocardiogram (ECG).

27. The method of claim 23 or claim 24, wherein for Type 2 diabetes the screening involves analysing one or more of blood glucose levels, urine glucose levels, glycated hemoglobin (HbA1c) levels, fructosamine levels or glucose tolerance of the subject.

28. A method for determining the need of a human subject for prophylactic anti-coronary artery disease therapy, anti-atrial fibrillation therapy or anti-Type 2 diabetes therapy comprising assessing the risk of the subject for developing coronary artery disease, atrial fibrillation or Type 2 diabetes using the method according to any one of claims 1 to 22.

29. A method for preventing or reducing the risk of coronary artery disease, atrial fibrillation or Type 2 diabetes in a human subject, the method comprising assessing the risk of the subject for developing coronary artery disease, atrial fibrillation or Type 2 diabetes using the method according to any one of claims 1 to 22, and if they are assessed as having a risk for developing coronary artery disease, atrial fibrillation or Type 2 diabetes administering anti-coronary artery disease therapy, anti-atrial fibrillation therapy or anti-Type 2 diabetes therapy, respectively.

30. An anti-coronary artery disease therapy, anti-atrial fibrillation therapy or anti-Type 2 diabetes therapy for use in preventing coronary artery disease, atrial fibrillation or Type 2 diabetes, respectively, in a human subject at risk thereof, wherein the subject is assessed as having a risk for developing coronary artery disease, atrial fibrillation or Type 2 diabetes using the method according to any one of claims 1 to 22.

31. The method according to any one of claims 28 to 30, wherein the anti-coronary artery disease therapy is selected from cholesterol lowering medication such as a statin, blood thinning medication such as aspirin, warfarin or rivaroxaban, a n-blocker, nitrates, or a calcium channel blocker.

32. The method according to any one of claims 28 to 30, wherein the anti-atrial fibrillation therapy is selected from cardioversion, a n-blocker, a calcium channel blocker, blood thinning medication such as warfarin, aspirin or rivaroxaban, or an antiarrhythmic drug such as quinidine, flecainide, propafenone, sotalol, dofetilide. or amiodar.

33. The method according to any one of claims 28 to 30, wherein anti-Type 2 diabetes therapy is selected from metformin, insulin, a sulfonylurea such as glimepiride, glyburide or glipizide, a meglitinide such as prandin or starlix, a thiazolidinedione such as rosiglitazone or pioglitazone, a DPP-4 inhibitor such as sitagliptin, saxagliptin, linagliptin or alogliptin, a GLP-1 receptor agonist such as exenatide, liraglutide, lixisenatide, albiglutide or dulaglutide, and an SGLT2 inhibitor such as forxiga, invokana or jardiance.

34. A method for stratifying a group of human subject's for a clinical trial of a candidate therapy, the method comprising assessing the individual risk of the subject's for developing coronary artery disease, atrial fibrillation or Type 2 diabetes using the method according to any one of claims 1 to 22, and using the results of the assessment to select subject's more likely to be responsive to the therapy.

35. A kit comprising at least two sets of primers for amplifying two or more nucleic acids, wherein the two or more nucleic acids comprise a polymorphism selected from any one of Tables 1 to 4, or a single nucleotide polymorphism in linkage disequilibrium with one or more thereof.

36. A genetic array comprising at least two sets of probes for hybridising to two or more nucleic acids, wherein the two or more nucleic acids comprise a polymorphism selected from any one of Tables 1 to 4, or a single nucleotide polymorphism in linkage disequilibrium with one or more thereof.

37. A computer implemented method for assessing the risk of a human subject developing coronary artery disease, atrial fibrillation or Type 2 diabetes, the method operable in a computing system comprising a processor and a memory, the method comprising:
receiving genetic risk data for the human subject, wherein the genetic risk data was obtained by a method according to any one of claims 1 to 22;
processing the data to obtain the risk of a human subject developing coronary artery disease, atrial fibrillation or Type 2 diabetes; and
outputting the risk of a human subject developing coronary artery disease, atrial fibrillation or Type 2 diabetes.

38. A computer implemented method for assessing the risk of a human subject developing coronary artery disease, atrial fibrillation or Type 2 diabetes, the method operable in a computing system comprising a processor and a memory, the method comprising:
receiving clinical risk data and genetic risk data for the human subject, wherein the clinical risk data and genetic risk data were obtained by a method according to any one of claims 12 to 16 or 20 to 22;
processing the data to combine the clinical risk data with the genetic risk data to obtain the risk of a human subject developing coronary artery disease, atrial fibrillation or Type 2 diabetes; and
outputting the risk of a human subject developing coronary artery disease, atrial fibrillation or Type 2 diabetes.

39. A system for assessing the risk of a human subject developing coronary artery disease, atrial fibrillation or Type 2 diabetes comprising:
system instructions for performing a genetic risk assessment of the human subject using the method according to any one of claims 1 to 22; and
system instructions to obtain the risk of a human subject developing coronary artery disease, atrial fibrillation or Type 2 diabetes.

40. A system for assessing the risk of a human subject developing coronary artery disease, atrial fibrillation or Type 2 diabetes comprising:
system instructions for performing a clinical risk assessment and a genetic risk assessment of the human subject using the method according to any one of claims 12 to 16 or 20 to 22; and
system instructions for combining the clinical risk assessment and the genetic risk assessment to obtain the risk of a human subject developing coronary artery disease, atrial fibrillation or Type 2 diabetes.
